(19) United States
(12) Patent Application Publication
(10) Pub. No.: US 2007/0099290 A1
(43) Pub. Date: May 3, 2007

(54) Customizable chip and method of manufacturing the same

(76) Inventors: Kazuhiro Iida, Tokyo (JP); Takashi Tanaka, Tokyo (JP)

Correspondence Address: SCULLY SCOTT MURPHY & PRESSER, PC, 400 GARDEN CITY PLAZA, SUITE 300, GARDEN CITY, NY, 11530, US

(21) Appl. No.: 10/570,423

(22) PCT Filed: Sep. 2, 2004
(86) PCT No.: PCT/JP04/12753

§ 371 Date: Mar. 2, 2006

(30) Foreign Application Priority Data
Sep. 2, 2003 (JP) 2003-309683

(51) Int. Cl. C12M 1/34

(52) U.S. Cl. 435/287.2; 977/900

(57) ABSTRACT

The present invention provides an analysis chip allowing customization in accordance with the items required for a given test, and method for producing the same. On a chip (313) there are formed dispensing channels (222) connected to respective detection reservoirs (223) and branching off from a main channel (221). Each branch channel (222) is provided with a flow control unit (314) responsible for an opening and closing of the channel. It is possible to achieve multiple analysis steps by appropriately setting an opening and closing of flow control units provided to an analysis portion.

### TECHNICAL FIELD

The present invention relates to a chip allowing customization of its configuration and the method of producing it.

### BACKGROUND ART

Recently, use of micro total analysis systems (μ-TAS) in which chemical operation such as the pretreatment of a sample, and its reaction, separation and detection is performed on a microchip has rapidly advanced. Such micro total analysis systems allows the use of a small amount of sample, imposes less burden to the environment, and enables high sensitivity analysis. Thus, if it were possible to apply such microchip based analysis to the clinical laboratory test in medicine, it would be highly advantageous because then the laboratory test could be easily performed on tiny amounts of samples.

Non-patent Document 1: David, S. J., Dwight, K. O., and Wayne, R. D. (Eds.), 2001, “Laboratory Test Handbook with Key Word Index,” 5th edition, Lexi-Comp Inc., Hudson, Ohio, pp. 77-80

### DISCLOSURE OF THE INVENTION

If micro total analysis systems were applied to the clinical laboratory test and the like, the number of tests achievable on a single chip would be limited by the size of the chip. Generally, the clinical laboratory test must determine many clinical parameters for a given purpose. For example, number of the clinical parameters determined by a general biochemical test is as many as 150 (Non-patent Document 1). If parameters related with tumor markers and allergens were added to the above, the number would rise up to 300. In addition, the clinical parameters vary according to the individual patient, and his/her disease condition.

If a laboratory of a certain clinical center meets such widely varied requirements from clinics by using chips, the laboratory will have to prepare such a huge number of chips as to enable the laboratory to meet widely varied combinations of clinical tests. Thus, such analysis system, could never been put into practice. In addition, a chip having one definite layout cannot meet two or more different combinations of tests.

The present invention was proposed in view of the above situation, and aims to provide a general-purpose analysis portion receptive to customization to meet a given test item, analysis chip, and production method thereof.

The term “chip” in the specification refers to a substrate having a function allowing one to perform predetermined operation on a sample placed thereupon. A chip according to the invention may have, for example, a channel groove on its surface so that a liquid sample can move along it. Movement of a liquid sample may be achieved via capillary action, or may be driven by external force such as electric field or pressure. If movement of a liquid sample along a channel is achieved by capillary action, it will be possible to dispense with the use of a unit for applying external driving force, and to move the liquid sample in a downstream direction solely dependent on the construction of the chip itself.

According to the invention, there is provided a chip comprising: a substrate; plural channels formed on the substrate; and a flow control portion which is provided to the plural channels and is capable of being closed, wherein closure of the flow control portion of a channel among the plural channels allows a sample to be guided to other channels.

The term “flow control portion capable of being closed” as used herein refers to the flow control portion capable of intercepting the passage of liquid by using a physical or a chemical treatment at the site where the flow control portion resides. Interception of liquid includes not only the complete occlusion of liquid but also partial passage of liquid downstream. Incidentally, if it is required to completely intercept the flow of liquid along a channel, the flow control portion is completely closed.

The chip of the invention is so constructed that the channel is provided with a closure-able flow control portion and when the flow control portion of one channel is closed, a liquid sample is guided to flow along the other channel. Because of this arrangement, it is possible to close desired channels and select desired channels for sample passage depending on the given use purpose of the chip or the property of the sample. Thus, it is possible to customize the configuration of the chip, despite that the structure itself of the chip is very simple.

It is possible to customize the configuration of a chip by setting an opening or a closing the flow control portions, thereby obtaining chips for the different test items. Thus, it is possible to readily obtain a chip whose channel configuration is most suitable for achieving the test required for a given patient or for determining the required clinical parameters. Since the chip includes flow control portions, it makes it unnecessary to prepare in advance a huge number of chips with different lay-outs corresponding to s huge number of test parameters. Because of the above-cited advantages, it is possible according to the invention to stably produce general-purpose chips receptive to customization at a low cost. Moreover, it is possible according to the invention to distribute a sample only to necessary channels by providing the flow control portion, which will allow the efficient use of the sample. Therefore, even if the amount of a sample is very small, it will be possible to reliably perform necessary tests on that sample. It is also possible to minimize the consumption of reagents required for analysis.

According to the invention, it is also possible to construct each flow control portion such that its closure can be modified at a post-processed state. This makes it possible to selectively close the desired flow control portion in accordance with the parameters required for a given test at a post-processing stage. Thus, it is possible to customize the configuration of a chip in accordance with an test item.

According to the invention, there is provided a chip comprising: a substrate; a sample introduction portion formed on the substrate; an analysis portion for analyzing a specified component contained in a sample introduced via the sample introduction portion; plural channels connecting the sample introduction portion with the analysis portions; and a flow control portion which is provided to the channels and is capable of being closed, wherein closure of the flow control portion of a channel among the plural channels allows a sample to be guided via other channel to the analysis portion.

In the invention, the analysis portion is connected via a flow control portion to a channel. At the analysis portion, analysis of a component in a sample is performed. When a sample is introduced via the sample introduction portion, the sample flows along a channel, and reaches an analysis portion for which the flow control portion connected thereto is open. When the chip further includes a pretreatment portion, a separation portion and a reaction portion, which will be described later, the chip is configured so that the sample passes through these portions before it reaches the analysis portion.

In the chip of the invention, the analysis portion or the separation portion which will be described later may work being driven by force applied externally, but they are preferably configured to work automatically, that is, separate a predetermined component and analyze the separated component in a specified order, driven by the inflow of sample. Realization of such automatic operation will be possible by utilizing capillary action or water-level difference as a driving force of a liquid sample. By utilizing capillary action, it is possible to make a sample introduced via the sample introduction portion flow through a channel automatically to an analysis portion to be analyzed there.

The chip of the invention includes plural channels each of which has its own flow control portion connected thereto. Because of this arrangement, it is possible to keep open a desired channel selectively among the plural channels while closing the flow control portions connected to the remaining channels. Thus, it is possible for a sample introduced via the sample introduction portion to pass through the desired route to reach an analysis portion.

According to the invention, there is provided a chip comprising: a substrate; a sample introduction portion formed on the substrate; analysis portions for analyzing a specified component contained in a sample introduced via the sample introduction portion; a branched channel for guiding the sample introduced via the sample introduction portion to the plural analysis portions; and a flow control portion which is provided to the channels and is capable of being closed, wherein closure of the flow control portion of a channel branched towards one of the analysis portion allows a sample to be guided to other of the analysis portions.

A chip of the invention has plural analysis portions each of which is in communication with a channel with its own flow control portion. Because of this arrangement, it is possible for a sample introduced via the sample introduction portion to be selectively transported only to a desired analysis portion. Thus, it is possible to supply a sample only to the analysis portions, among all the analysis portions for a plurality of test items, responsible for the determination of parameters required for a given test item. Accordingly, even if a sample is small in amount, it is possible to reliably select and determine the parameters required for a given test.

According to the invention, the flow control portion may be constructed such that its closure is achieved by clogging a part of the channel. Alternatively, in the chip according to the invention, closure of the flow control portion may be achieved by hydrophobizing the surface of the channel. These arrangements will ensure the reliable closure of the flow control portion.

The chip according to the invention may further include a separation portion which includes a part of a channel, and separate a component contained in a sample introduced via the sample introduction portion to guide the component to an analysis portion. Because of this arrangement, it is possible to reliably separate a predetermined component from a sample, and to supply the component to a selected analysis, which will increase the analysis sensitivity.

The chip according to the invention may further include a pretreatment portion upstream of the separation portion which will apply specified pretreatment on a sample introduced via the sample introduction portion. This arrangement will make it possible to apply pretreatment on a sample on the chip. Accordingly, it will be possible to render the sample more suitable for analysis subsequently performed. Further, in the invention, the pretreatment potion has the aforementioned flow control portion. Because of this arrangement, it will be possible to customize pretreatments performed by preparing a number of layouts suitable for a plurality of pretreatments and closing appropriate flow control portions at a post-processing stage. Thus, it is possible to selectively perform pretreatments as appropriate in accordance with a given sample.

In the chip in the invention, the pretreatment portion includes a reservoir, and a liquid switch portion provided downstream of the reservoir which controls the flow of a liquid sample from the pretreatment portion to the separation portion, wherein the liquid switch portion includes a damming portion for damming a liquid in the reservoir, and a trigger channel which communicates with a channel close to the damming portion, and guides the liquid to the damming portion, and wherein a flow control portion may be provided to the trigger channel. Through this arrangement it is possible to more stably apply desired pretreatment to a sample.

The chip in the invention may further include a reaction portion where a component separated at the separation portion undergoes a specified reaction. Through this arrangement it is possible to analyze a sample under condition more suitable for measurement. The reaction portion may also include the aforementioned flow control portion. Through this arrangement it is possible to customize the kinds of reactions performed at the reaction portion by preparing in advance reaction portions responsible for the occurrence of various reactions, and closing the flow control portion at a post-processing stage. Therefore, it is possible to allow the reactions to occur that are selected appropriately in accordance with a sample.

In a chip of the invention, the reaction portion includes a reservoir and a liquid switch portion provided downstream of the reservoir, the liquid switch portion includes a damming portion for damming the flow of a liquid in the reservoir, and a trigger channel which communicates with a channel close to the damming portion, and guides the liquid to the damming portion, and a flow control portion may be provided to the trigger channel. Through this arrangement, it is possible to allow a sample to undergo desired reactions sequentially.

In the invention, the flow control portion has a larger width than a channel connected thereto so that it can intercept the passage of flow through the channel. Through this arrangement, it is possible to selectively close a channel.

In the invention, part of the flow control portion may be open to outside. For example, a chip of the invention may further include a lid for covering the top surface of the channel such that when the lid is put in place there is formed an opening over the flow control portion. Through this arrangement it is possible to reliably close the flow control portion through the opening at a post-processing step.

According to the present invention, there is provided a chip including a substrate, and plural channels formed on the substrate, wherein some of the plural channels are closed.

In the chip of the invention, since a piece of plural flow control portions are closed, movement of sample to downstream through the closed portion is prevented. Because of this, passage of sample only to selected open channels is permitted.

According to the present invention, there is provided a method for producing a chip including preparing a substrate on which plural channels are formed, and closing some of the channel.

The method for producing a chip in the invention includes the closing a piece of a channel. According to this method, it is possible to stably produce chips in which movement of sample to downstream through the closed portions will be securely prevented. Therefore, it is stably reproduce the configuration of passage on the substrate appropriate for a sample.

In the method for producing a chip in the invention, the closing a channel may include hydrophobizing a part of the channel. By so doing it is possible to further enhance the closure of a part of the channel. Therefore, it is possible to more stably produce customized chip.

In the method for producing a chip in the invention, the closing a channel may include deforming a part of the channel to intercept it. By so doing it is possible to more reliably close the channel.

In the method for producing a chip in the invention, the closing a channel may include a sealing a part of the channel. It is possible to reliably intercept the flow of liquid through the channel by sealing a part of the channel. This ensures the reliable closure of a part of the channel. Sealing a channel used herein means closing the cross-section of a channel with a sealing material.

In the invention, the portion close to the damming portion may be placed at the damming portion itself or downstream of the damming portion. Through this arrangement it is possible to more reliably intercept the flow of liquid through the passage.

The above description has been given on the premise that closure of the flow control portion provided to a channel leads to the passage of sample to the other channels. According to an embodiment of the invention, however, it is also possible to prepare a group of flow control portions capable of opening, and to open some of the flow control portions so that sample can pass to the channels connected to those open flow control portions. Through this arrangement, it is possible to select channels to be opened according to the requirement from a given test and to only open the selected channel at a post-processing stage, so that sample may be guided to those selected channels. Thus, it is possible to customize the configuration of a chip according to the reagents used and analysis items.

To execute at site a test appropriate for a given patient, it is necessary to have a large-scale facilities. If a comparatively small clinical center or laboratory wants to execute a test, by preparing in advance general-purpose chips applicable to widely used items and combination analysis thereof, and customizing the configuration of the chip as appropriate, the test required for a given patient can be performed in the small-scale facilities.

For example, if a small clinical center prepares in advance one or more kinds of general purpose chips, even if it is difficult to own relatively large scale facilities, by appropriately setting an opening and a closing of the flow control portions, or setting predetermined reagents to the chip as appropriate, so as to customize the configuration of chip in accordance with the medical condition and its course of a patient. Therefore, it is possible to give a simple and quick analysis suitable for a given patient quickly at site. The analysis portion of a chip and chip itself enabling such customization will be described below.

According to the present invention, there is provided a general-purpose analysis portion comprising a main channel; a reservoir; a channel connecting the reservoir and the main channel; a damming portion provided to the channel for damming a liquid in the reservoirs; a trigger channel in communication at or close to the damming portion with the channel, the trigger channel being for guiding the liquid to the damming portion; a liquid switch portion including the damming portion and the trigger channel; a closing switch for closing the channel; a lag channel provided to trigger channel or channel; and a flow control portion for setting an opening and a closing of the channel or the trigger channel.

In the general-purpose analysis portion of the invention, sample moves past the main channel and channel to reach the reservoir and be used for a predetermined analysis. The constitutional member of the general-purpose analysis portion of the invention may be standardized according to the envisioned analysis items. Thus the analysis portion may be preferably used as the general-purpose analysis portion. In the general-purpose analysis portion of the invention, a trigger channel is provided for the purpose of setting an opening and a closing of the channel or the trigger channel. When at least a part of the flow control portion is opened, liquid can pass through the flow control portion, while a flow control portion is completely blocked, liquid cannot pass though the flow control portion. Thus, it is possible to set the route of the liquid by setting the opening and the closing of the flow control portions. Thus, the chip in the invention can be customized in accordance with a given sample for the analysis or a given test.

The liquid switch portion is a switching mechanism for controlling a flow of a liquid such as sample or a buffer in the channel. In the liquid switch portion, a liquid flowing along the channel is intercepted at its damming portion. The damming portion may be so constructed as to absorb a liquid thereby holding the liquid. Alternatively, the damming portion in itself may be lyophobic to the flowing liquid, and may be so constructed that the liquid is intercepted on its upstream side. The liquid switch portion includes the trigger channel. Liquid intercepted by damming portion goes beyond the damming portion to flow downstream, when it contact with the liquid passing through trigger channel.

Since the liquid switch portion is provided to the channel, introduction of a sample from the channel to the reservoir is controllability achieved. With a general-purposed analysis portion as described above, it is possible to obtain desired analysis result because predetermined reactions necessary for analysis are allowed to stably occur under the desired condition. It is also possible by providing a liquid switch portion to drive a plurality of process steps by capillary action sample once a sample is introduced into the system, without resorting to any external supporting apparatus.

The lag channel is provided to a predetermined portion of the channel or the trigger channel for producing a delay in the time distance of a sample flowing from one region to another region. Introduction of a lag channel further facilitate the condition of the reaction required for the analysis, and the like.

The closing switch has a valve structure configured so that it is closed when the predetermined amount of the liquid is introduced into the channel or the trigger channel to which the closing switch is. In the general-purpose analysis portion in the invention, thanks to the valve structure, only a certain predetermined amount of liquid is permitted to flow through a given channel or trigger channel to the reservoir and also counter current of the liquid is prevented.

In the general-purpose analysis portion of the invention, the reservoir may hold a reagent. Then, it is possible to more efficiently execute analysis using the reagent in the general-purpose analysis portion.

The general-purpose analysis portion of the invention may include two of the reservoirs, one of the liquid switch portion, one of the closing switch, one of the delaying channel, and one or two of the flow control portions. Alternatively, the general-purpose analysis portion of the invention may include five of the reservoirs, two or more of the liquid switch portions, two or more of the closing switches, two or more of the delaying channels, and two or more of the flow control portions.

The general-purpose analysis portion in the invention may take varied configurations. Typical configuration is, for example, divided into the following three types: a first type general-purpose analysis portion described in the following (I), a second type general-purpose analysis portion described in the following (II), and a third type general-purpose analysis portion described in the following (III). The general-purpose analysis portion described in the following (I) to (III) have the aforementioned main channel and the aforementioned channel, and further has the following structure.

(I) First Type General-Purpose Analysis Portion

It includes one of said reservoir and one of the flow control portion. It may further have one of the closing switch.

(II) Second Type General-Purpose Analysis Portion

It includes at least two of the reservoirs, at least one of the flow control portion, at least one of the closing switch, at least one of the liquid switch, and at least one of the lag channel.

(III) Third Type General-Purpose Analysis Portion

It includes at least five of the wells, two or more of the liquid switch portions, two or more of the lag channels, two or more of the flow control portions, and one or more of the closing switch.

The structure of the above (I), (II) and (III) can mainly be used to an analysis of a predetermined component in a sample with one-step reaction, two-step reaction, and three-step reaction, respectively.

According to the present invention, there is provided a chip including a substrate, and a general-purpose analysis portion formed on the substrate. The chip of the invention, because of its including the aforementioned general-purpose analysis portion, is amenable to standardization, can be customized according to individual needs, and suitably used as a general-purpose chip. According to the inventive chip, it is possible to customize the configuration of a chip by opening/closing the flow control portions according the parameters required for a given test. Thus, it is possible to reliably perform a required test using only a minimum amount of reagent and sample.

The chip of the invention may include a plurality of the general-purpose analysis portions. By so doing, it is possible to standardize the configuration of the chip for plural analyses, which further enhances the utility of the chip. It is also possible to customize the configuration of a chip to match a given test for an examiner by setting the opening/closing-state of the flow control portions at a post-processing stage. In other words, the chip of the invention can be customized in accordance with the personal needs of individual users.

The configuration of a chip of the invention can be modified in widely different manners, but the chip may configured as following and the different configurations of a chip may be classified according to the type of disease.

(i) Chip for the Diagnosis of Diabetes Set

Provided is a configuration which has an analysis portion including at least one of the third type general-purpose analysis portion and at least three of the first or the second type general-purpose analysis portions, at least one has a reservoir holding a reagent, wherein, when the third type general-purpose analysis portion has the reagent, the reagent being necessary for the detection of anti glutamate decarboxylase antibody, and when the first or the second general-purpose analysis portion has the reagent, the reagent being stored is necessary for determining any one, or two or more test items selected from the group consisting of hemoglobin A1c, 1,5-anhydro-D-glucitol, and glycoalbumin.

(ii) Chip for the Diagnosis of Obesity Set

Provided is a configuration which has an analysis portion including at least eight of the first or the second type general-purpose analysis portions, at least one of the second type general-purpose analysis portions has a reservoir holding a reagent, wherein the reagent is necessary for determining any one, or two or more test items selected from the group consisting of aspartate aminotransferase, alanine aminotransferase, γ-glutamyl transpeptidase, total cholesterol, neutral fatty acid, HDL cholesterol, fasting blood sugar (glucose), and hemoglobin A1c.

(iii) Chip for the Diagnosis of Hyperlipidemia

Provided is a configuration which has an analysis portion including at least nine of the first or the second type general-purpose analysis portions, at least one of the nine of the first or the second type general-purpose analysis portions has a reservoir for holding a reagent, wherein the reagent is necessary for determining any one, or two or more test items selected from the group consisting of remnant lipoprotein cholesterol, LDL-cholesterol, lipoprotein a, apoprotein A-I, apoprotein A-II, apoprotein B, apoprotein C-II, apoprotein C-III, apoprotein E, creatine phosphokinase, aspartate aminotransferase, alanine aminotransferase, and γ-glutamine transpeptidase, and necessary for determining two or more test parameters.

In the above chip (iii), at least one of the nine second type general-purpose analysis portions has a reservoir for holding a reagent, wherein the reagent is necessary for determining any one, or two or more test items selected from the group consisting of remnant lipoprotein cholesterol, LDL-cholesterol, lipoprotein a, apoprotein A-I, apoprotein A-II, apoprotein B, apoprotein C-II, apoprotein C-III, and apoprotein E. The chip may further include, in addition to the nine general-purpose analysis portions, a general-purpose analysis portion that has a reservoir for holding a reagent, wherein the reagent is necessary for the detection of creatine phosphokinase, aspartate aminotransferase, alanine aminotransferase, and γ-glutamine transpeptidase. With this chip, it is possible to collect more accurate data necessary for hyperlipidemia including when the patient internally takes a therapeutic agent. The above chip (iii) may include an analysis portion including at least 13 of the first or the second general-purpose analysis portions.

(iv) Chip for the Diagnosis of Disordered Hepatic Function

Provided is a configuration which has an analysis portion including at least two of the third type general-purpose analysis portion, and at least eight of the first or the second type general-purpose analysis portions, at least one of the third type general-purpose analysis portion has a reservoir for holding a reagent, wherein, when the third type general-purpose analysis portion has the reagent, the reagent is necessary for determining any one, or two or more test items selected from the group consisting of HBs antibody, and HCV antibody, and when the first or the second type general-purpose analysis portion has the reagent, the reagent necessary for determining any one, or two or more test items selected from the group consisting of alkaline phosphatase, lactate dehydrogenase, total protein, albumin, agent for zinc phosphate turbidity test, agent for thymol turbidity test, choline esterase, and total bilirubin.

(v) Chip for the Diagnosis of Nephrosis

Provided is a configuration which has an analysis portion comprising at least seven of the first or the second type general-purpose analysis portions, at least one of the first or the second type general-purpose analysis portions has a reservoir for holding a reagent, wherein the reagent is necessary for determining any one, or two or more test items selected from the group consisting of total protein, albumin, urea nitrogen, creatinine, sodium ion, potassium ion, and chlorine ion.

(vi) Chip for the Diagnosis of Hypertension

Provided is a configuration which includes an analysis portion including at least two of the third type general-purpose analysis portion, and at least five of the first or the second type general-purpose analysis portions, at least one has a reservoir for holding a reagent, wherein, when the third type general-purpose analysis portion has the reagent, the reagent is selected from the group including agent for determining renin activity, and aldosterone, and when the first or the second type general-purpose analysis portion has the reagent, the reagent is necessary for determining any one, or two or more test items selected from the group consisting of nitrogen urea, creatinine, sodium ion, potassium ion, and chlorine ion.

(vii) Chip for the Diagnosis of Anemia Set

Provided is a configuration which includes an analysis portion comprising at least two of the third type general-purpose analysis portion, and at least two of the first or the second type general-purpose analysis portions, at least one has a reservoir for holding a reagent, wherein, when the third type general-purpose analysis portion has the reagent, the reagent is necessary for determining any one, or two or more test items selected from the group consisting of vitamin B12, and folic acid, and when the first or the second type general-purpose analysis portion has the reagent, the reagent is necessary for determining any one, or two or more test items selected from the group consisting of.

(viii) Chip for the Diagnosis of Gout

Provided is a configuration which includes an analysis portion comprising at least one the first or the second type general-purpose analysis portions, at least one holds a reagent necessary for the detection of uric acid.

(ix) Chip for the Diagnosis of Disorder of Thyroid Function

Provided is a configuration which includes an analysis portion comprising at least three of the third type general-purpose analysis portions, at least one of the third type general-purpose analysis portions has a reservoir for holding a reagent, wherein the reagent is necessary for determining any one, or two or more test items selected from the group consisting of triiodothyronine, thyroxine, and thyroid gland stimulating hormone.

(x) Chip for Determining the Activity of Adrenal

Provided is a configuration which includes an analysis portion comprising at least one of the third type general-purpose analysis portions which has a reagent necessary for the detection of cortisol.

The chip of the invention may be so constructed as to have the same number of general-purpose analysis portions with that for a sample, and may be subjected to the same analysis task using a standard solution instead of the sample. The result obtained by the chip from the standard solution may be compared with the result obtained by the test chip using a sample. The comparison will further enhance the accuracy of the diagnosis by the test chip.

The present invention provides an analysis chip allowing customization of the chip layout in accordance with test items, general-purpose analysis portion, and method for producing such a chip and analysis portion.

### BRIEF DESCRIPTION OF THE DRAWINGS

The object of the invention described above, and other objects, and its features and advantages will be further apparent by reading the following description of preferred embodiments with reference to the attached drawings.

FIG. 1 shows a block diagram for representing the functional components of a chip representing an embodiment of the invention.

FIG. 2 shows the configuration of a chip capable of achieving the functions as represented in FIG. 1.

FIG. 3 shows a cross-section of the chip shown in FIG. 2 cut along line A-A′.

FIG. 4 shows a cross-section of the chip of FIG. 2 cut along line B-B′.

FIG. 5 shows a cross-section of the chip of FIG. 2 cut along line B-B′.

FIG. 6 shows a cross-section of the chip of FIG. 2 cut along line C-C′.

FIG. 7 illustrates how a flow control portion of a chip is closed according to an embodiment of the invention.

FIG. 8 further illustrates how the flow control portion of a chip is closed according to the embodiment of the invention.

FIG. 9 still further illustrates how the flow control portion of a chip is closed according to the embodiment of the invention.

FIG. 10 shows a block diagram for representing the functional components of a chip representing another embodiment of the invention.

FIG. 11 shows the configuration of a chip capable of achieving the functions as represented in FIG. 10.

FIG. 12 shows the structure of measurement unit of the chip shown in FIG. 11.

FIG. 13 further shows the structure of measurement unit of the chip shown in FIG. 11.

FIG. 14 shows a diagram for schematically showing the structure of a measuring apparatus representing an embodiment.

FIG. 15 shows how a chip is inserted into the measuring apparatus shown in FIG. 14.

FIG. 16 shows the structure of a measuring apparatus representing an embodiment.

FIG. 17 shows the structure of a chip representing an embodiment.

FIG. 18 shows a cross-section of the chip shown in FIG. 17 cut along line D-D′.

FIG. 19 shows a block diagram for representing the functional components of a chip representing an embodiment of the invention.

FIG. 20 shows a block diagram for representing the functional components of another chip representing an embodiment of the invention.

FIG. 21 shows a diagram for representing the components of a chip further including a separation unit representing an embodiment.

FIG. 22 shows the structure of separation region of the chip shown in FIG. 21.

FIG. 23 illustrates the method of molecular separation occurring at the separation region shown in FIG. 22.

FIG. 24 shows the structure of a chip representing an embodiment.

FIG. 25 shows the structure of the mixing portion of the chip shown in FIG. 24.

FIG. 26 shows the structure of the mixing portion of the chip shown in FIG. 24.

FIG. 27 shows enlarged views of the liquid switch portion shown in FIG. 26.

FIG. 28 shows the damming portion of the liquid switch portion shown in FIG. 26.

FIG. 29 shows the structure of the trigger channel of the chip representing an embodiment.

FIG. 30 shows a block diagram for representing the functional components of a chip representing an embodiment of the invention.

FIG. 31 shows a block diagram for representing the functional components of another chip representing an embodiment of the invention.

FIG. 32 shows the structure of a chip representing an embodiment.

FIG. 33 shows the pretreatment unit of the chip shown in FIG. 32.

FIG. 34 shows a block diagram for representing the functional components of a chip representing an embodiment of the invention.

FIG. 35 shows a block diagram for representing the functional components of another chip representing an embodiment of the invention.

FIG. 36 shows the structure of a chip representing an embodiment.

FIG. 37 shows the structure of the reaction unit of the chip shown in FIG. 36.

FIG. 38 shows the structure of the detection unit of a chip representing the sixth embodiment.

FIG. 39 shows a schematic diagram for showing an exemplary chip manufacturing system representing ninth embodiment.

FIG. 40 shows a schematic diagram for showing another exemplary chip manufacturing system representing an embodiment.

FIG. 41 illustrates how the flow of sample through a flow control unit is intercepted according to an embodiment of the invention.

FIG. 42 further illustrates how the flow of sample through the flow control unit is intercepted according to the embodiment of the invention.

FIG. 43 shows the organization of a chip manufacturing system representing an embodiment.

FIG. 44 shows the procedures for the manufacture of a chip representing an embodiment.

FIG. 45 shows the structure of separation unit of the chip shown in FIG. 21.

FIG. 46 shows the structure of separation region of the chip shown in FIG. 21.

FIG. 47 shows plane views for showing the structure of a trigger channel of a chip representing an embodiment.

FIG. 48 shows plane views for showing the structure of a trigger channel of a chip representing an embodiment.

FIG. 49 shows a plane view for showing the structure of the detection unit of a chip representing an embodiment.

FIG. 50 shows a plane view for showing the structure of the detection unit of a chip representing the fifth embodiment.

FIG. 51 shows sectional views for showing the structure of a chip having a detection unit as depicted in FIG. 50.

FIG. 52 shows a plane view for showing the structure of the closing switch in the detection unit of a chip representing the sixth embodiment.

FIG. 53 shows a plane view for showing the structure of a liquid switch portion of a chip having a detection unit as depicted in FIG. 50.

FIG. 54 shows a list of principal test items required for a recheck test, involved measuring methods, and applicable class of reaction unit.

FIG. 55 shows another list of principal test items required for a recheck test, involved measuring methods, and applicable class of reaction unit.

FIG. 56 shows yet another list of principal test items required for a recheck test, involved measuring methods, and applicable class of reaction unit.

FIG. 57 shows yet another list of principal test items required for a recheck test, involved measuring methods, and applicable class of reaction unit.

FIG. 58 shows a plane view for showing the structure of the detection unit of a chip representing an embodiment.

### BEST MODE FOR CARRYING OUT THE INVENTION

The present invention will be described by means of embodiments with reference to the attached drawings. Throughout the drawings, every common element is represented by the same symbol, and its explanation will be not be repeated as appropriate.

At first, the basic structure of a chip receptive to customization upon which analysis of a sample will be made will be described in first and second embodiments. A chip includes, as its basic components, a sample introduction unit, flow control unit, and analysis unit. The analysis unit is a site where analysis of a component separated from a sample is performed. The analysis unit may also act as a detection unit when reaction is allowed to occur there to generate a visible product that indicates the presence of a target component. The analysis unit may also act as a measurement unit for storing a sample component that will serve as a subject to be analyzed by an external measuring device. The first embodiment represents a chip where the analysis unit serves as the detection unit while the second embodiment a chip where the analysis unit serves as the measurement unit. The structural details of the flow control unit will be described in the following embodiments.

### First Embodiment

This embodiment represents a chip that enables the determination of some parameters selected from available plural test items. The chip includes plural detection units responsible for the determination of respective test items, which will also serve as analysis portions. Each detection unit is in communication with a dispensing channel to which is provided a flow control unit which determines whether downward flow of liquid through the dispensing channel should be permitted or not. The chip is configured such that a sample is guided only to the detection units responsible for the determination of test items required for a given test, by setting an opening and closing of the flow control units connected to relevant detection units.

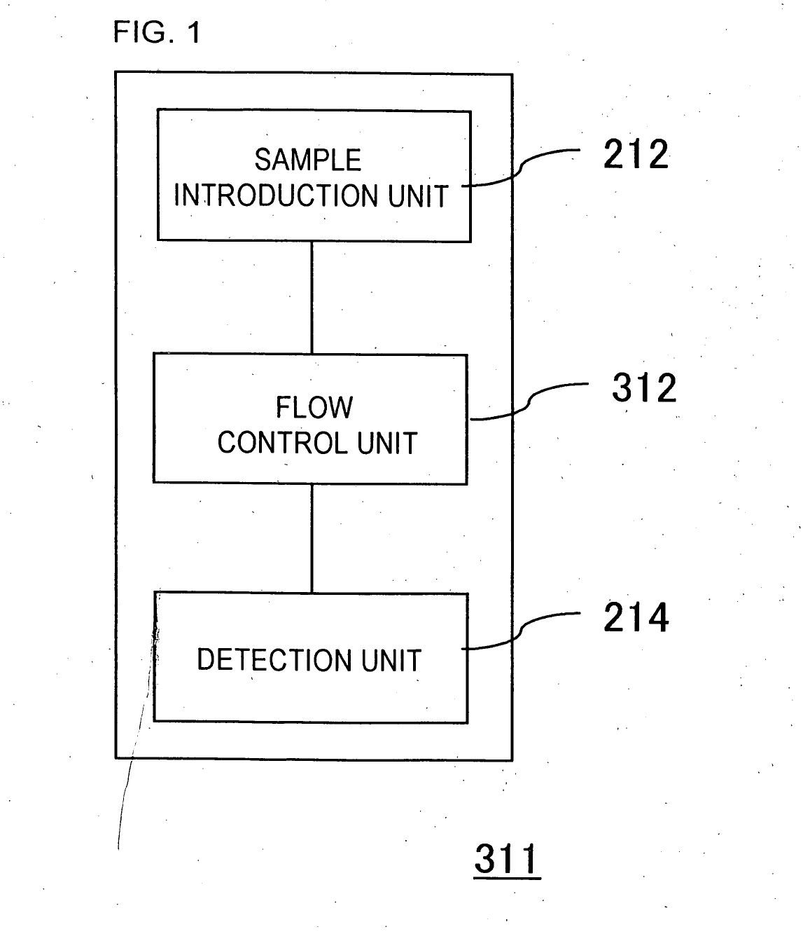

FIG. 1 shows a block diagram for representing the functional components of a chip representing the first embodiment. The chip shown in the FIG. 1 allows one to analyze the elements of a sample, and includes a sample introduction unit 212, a flow control unit 312, and a detection unit 214. The chip 311 includes a substrate made, for example, of silicon, glass, quartz, various plastic materials, or elastic materials such as rubber, and has an analysis system formed on its surface. The analysis system may be obtained by forming plural grooves on a surface of the substrate, placing a top member over the grooves to seal them, and providing, to the resulting closed channels, members and channels connecting those members so as to achieve the functions as shown in FIG. 1.

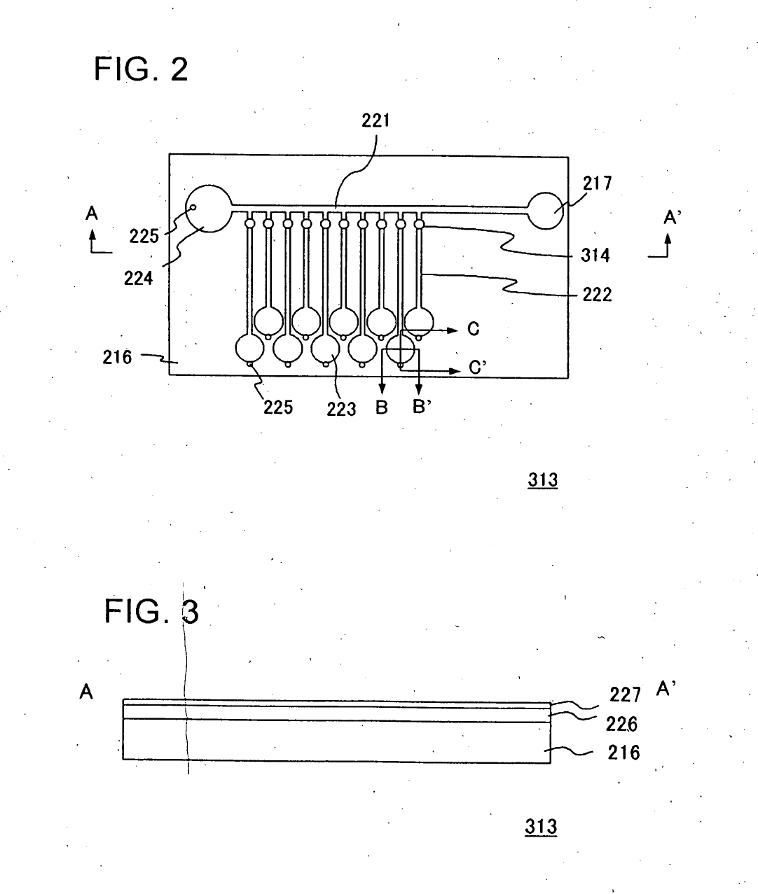

FIG. 2 shows an exemplary configuration of a chip 311 capable of achieving the functions as represented in FIG. 1. The chip 313 shown in FIG. 2 includes, on a surface of a substrate 216, an inlet 217, a main channel 221, dispensing channels 222, flow control units 314, detection reservoirs 223, and a reservoir 224. FIG. 3 shows a cross-section of the chip shown in FIG. 2 cut along line A-A′. FIG. 3 shows only the layered structure of the chip including substrate 216, lid 226 and seal 227 with the constitutive members such as the main channel 221 not being represented. The chip 313 has a lid 226 over the substrate 226. To the lid 226, are provided vent holes 225 communicating with the reservoir 224 and respective detection reservoirs 223. The top surface lid 226 may be sealed with a seal 227 to prevent the entry of contaminants including dust. FIG. 3 represents a cross-section of a chip having the seal.

The substrate 216 may have an area of 3 to 10 cm×2 to 7 cm, for example. It may have a thickness, for example, of 0.5 mm to 1 cm. The lid 226 may be made of the same material with the one constituting the substrate 216. The surface of substrate 216 and bottom surface of the lid 226, that is, the joining surfaces to the substrate 216 are preferably hydrophilic. Because then it is possible for a sample to be introduced into and move within the chip 313 via capillary action, without requiring the use of an external driving unit such as a pump or electrodes, and thus it is possible to simplify the structure of the system.

Each of the main channel 221 and dispensing channels 222 may have a cross-section of 100 μm in width and 20 μm in depth. Each detection reservoir 223 may have a cylindrical shape with a diameter of 2 mm, or rectangular shape with the side of 2 mm. The detection reservoir 223 may have the same depth with that of the dispensing channel 222, or may be as large as but slightly smaller than the thickness of the substrate. When optical measurement is performed on the detection reservoir 223 by radiating a beam onto the reservoir in a direction in parallel with the thickness of chip 313, and detecting the presence of a target component in a sample, it is preferable for the detection reservoir 223 to have a depth about as large as that of the dispensing channel 222, or as large as but slightly smaller than the thickness of the substrate, because then it is possible to lengthen the optical path, thereby enhancing the sensitivity of optical detection.

The vent hole 225 of a detection reservoir 223 is not necessarily provided on the top of the well, as long as the vent hole 225 is provided close to the well 223 in communication with the latter. The vent hole 225 may have a round cross-section, for example, with a diameter of 50 μm to 1 mm. By providing a vent hole to each detection reservoir 223, it is possible to securely guide fluid to the well 223. The surfaces of each vent hole 225 and its surrounds are preferably hydrophobic. Because then it is possible to prevent liquid poured into the detection reservoir 223 from leaking through the vent hole 225. When the leak of liquid from the detection reservoir 223 is safely prevented, it is possible to ensure that liquid stored in that detection reservoir 223 has a constant volume as specified. Loss of the sample is also prevented.

The seal 227 may be made of any appropriate material, as long as it can be peeled off prior to the use of a chip 313 to which it is applied. For example, a sheet obtained by applying an emulsion-based adhesive such as vinyl acetate on any one chosen from various plastic materials, may be used. Alternatively, an epoxy-based or silicone-based adhesive may be used.

The inlet 217 corresponding to a sample introduction unit 212 is a portion through which a sample is introduced into the system, and takes the shape of a reservoir or a well on the chip 313. The inlet 217 may be formed by preparing a cylindrical cavity with a diameter of about 3 mm in a substrate, preparing a circular throughhole of the same size on a lid 226 at a corresponding site, and putting the lid in place over the substrate.

The reservoir 224 serves as a waste reservoir and may be obtained by preparing a cylindrical cavity with a diameter of about 5 mm in a substrate, preparing a vent hole 225 on a lid 226 at a corresponding site, and putting the lid in place over the substrate. The surfaces of vent hole 225 of reservoir 224 and its surrounds are preferably made hydrophobic like those of vent holes 225 of detection reservoirs 223. The vent hole 225 of reservoir 224 is not necessarily provided on the top of the reservoir, as long as the vent hole 225 is provided close to reservoir 224 in communication with the latter. The vent hole 225 may have a round cross-section, for example, with a diameter of 50 μm to 2 mm. The vent hole in question may have a round cross-section whose diameter is larger than the corresponding diameter of vent holes 225 of adjacent detection reservoirs 223.

When a chip 313 is used for analysis, and has a seal 227 attached thereto, at first the seal 227 is removed. When the seal 227 is removed, the inlet 217 and vent holes 225 become open and exposed to external air. Then, a sample is introduced into the now opened inlet 217. The sample introduced is guided via capillary action into the main channel 221.

Various components contained in the sample that moves along the main channel 221 are distributed to dispensing channels 222 communicating with the main channel 221 to be guided to plural respective detection reservoirs 223 where they accumulate. The detection reservoirs 223 shown in FIG. 2 correspond to the detection unit 214 shown in FIG. 1. The dispensing channels 222 and detection reservoirs 223 may be provided in their number as needed on a substrate 216.

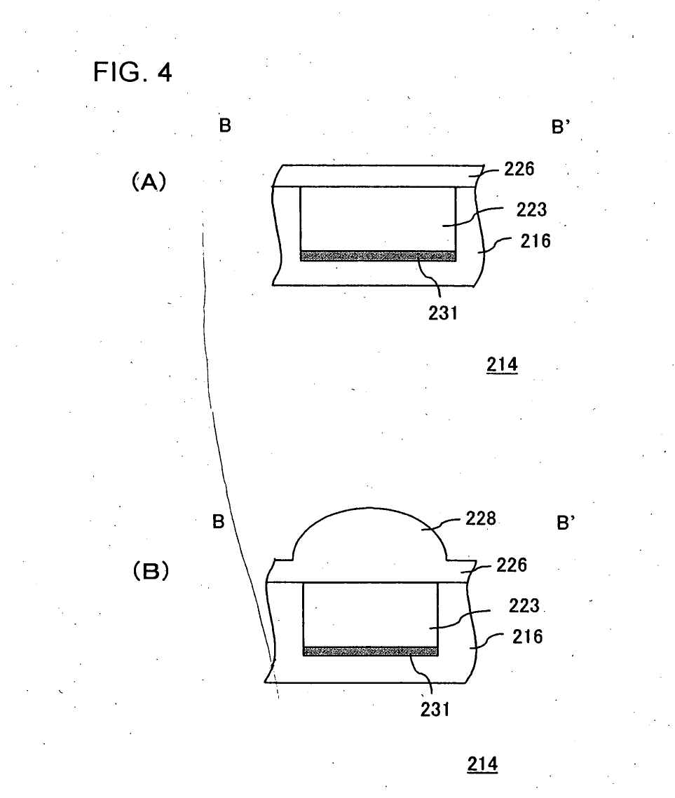

FIGS. 4A and 4B show a cross-section of the chip of FIG. 2 cut along line B-B′ with attention being paid to the detection unit 214 including a detection reservoir 223 as the main constructional member. Each of the detection reservoirs 223 shown in FIGS. 4A and 4B contains a detection reagent 231 in its bottom. The detection reagent 231 may be chosen such that when it is brought into contact with a specified component contained in a sample, it can give a color, emit light, change its color, be bleached, lose its light, or the like as a result of the interaction with the component. When a sample, after passing through a separation region 218, reaches a detection reservoir 223, a detection reagent 231 is dissolved or dispersed in a mobile phase, and undergoes the predetermined reaction with the sample in detection reservoir 223, and the reaction is detected. When a chip has plural detection reservoirs 223 like the chip 313 shown in FIG. 2, one detection reservoir 223 may be put aside from the analysis using a detection reagent 231, but used as a reference.

The detection reservoir shown in FIG. 4A is configured such that, when the sample develops a color and the like as a result of reaction, it can be visually confirmed through the lid 226. The detection reservoit shown in FIG. 4B has a lid 226 protruded in profile which serves as a micro-convex lens 228 when viewed from top for enlarging the image of the interior of detection reservoir 223. With this arrangement, it is possible to precisely check visually the change of sample solution such as development of color, emission of light, change of color, or bleaching or disappearance of color. Moreover, even if the detection reservoir 223 has a very limited area, it will be possible to reliably check the change of sample solution within such as development of color, emission of light, change of color, or bleaching or disappearance of color. This allows the amount of a sample required for analysis to be minimized.

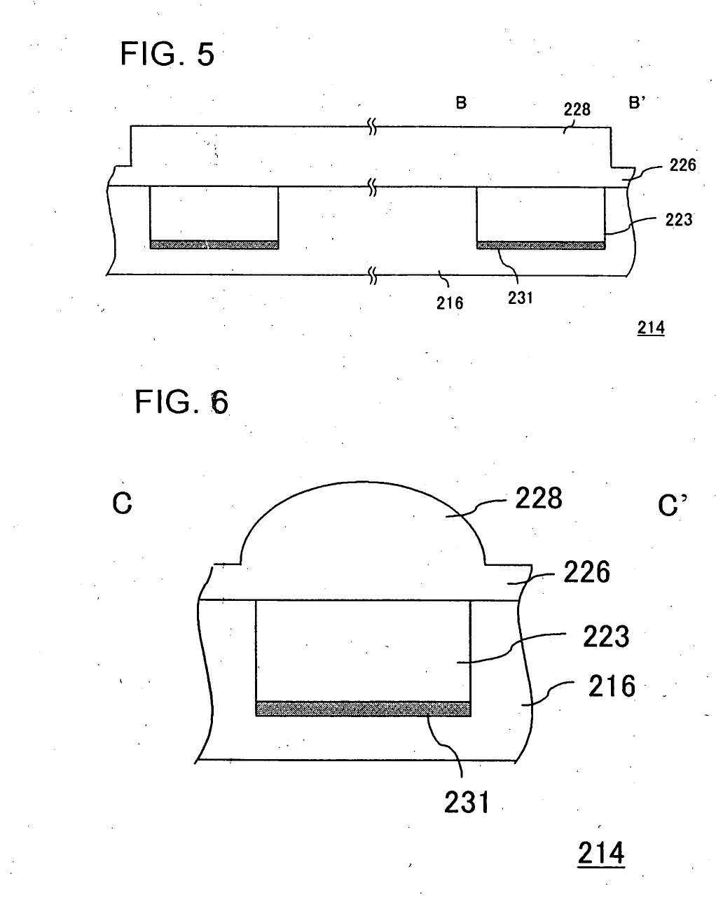

FIGS. 5 and 6 show the structure of detection unit 214 representing another embodiment. FIG. 5 shows a cross-section of the chip of FIG. 2 cut along line B-B′, and FIG. 6 a cross-section of the chip of FIG. 2 cut along line C-C′. As shown in FIGS. 5 and 6, a single micro-convex lens 228 may be extended to cover two or more detection reservoirs 223. In this case, the micro-convex lens 228 may be, for example, of half-cylinder shape which will simplify the structure of lid 226.

It is possible to supply a different detection reagent 231 to each of the plural detection reservoirs 223. By virtue of this arrangement, it is possible, even when a sample contains plural components, to detect tcomponents via the corresponding detection reactions by using a single chip. Therefore, it is possible to determine multiple parameters required for a given test using a minimum amount of sample.

Turn again to the chip 313 of FIG. 2. From main channel 221 are sequentially branched off plural dispensing channels 222. Since the dispensing channel 222 has a smaller cross-section than the main channel 221, a sample is withdrawn via capillary action sequentially from the upstream dispensing channel 222 with their detection reservoir 223 to the downstream dispensing channels 222 with their detection reservoir 223.

Each dispensing channel 222 is equipped with a flow control unit 314. The flow control unit 314 is so constructed as to close the dispensing channel 222 connected thereto as needed, thereby intercepting the flow of sample therethrough to downstream side. Therefore, a sample is withdrawn only through dispensing channels 222 whose flow control units 314 are kept open so that selected detection reactions can occur at the detection reservoirs 223 connected to the dispensing channels 222. On the contrary, the sample is not allowed to flow through the dispensing channels 222 whose flow control units 314 are kept closed and thus any detection reaction does not occur in the detection reservoirs 223 connected to those closed dispensing channels.

Unnecessary sample left after its necessary fraction being supplied through all the open dispensing channels 222 whose flow control units 314 are kept open to respective detection reservoirs 223 is evacuated into the reservoir 224.

By providing flow control units 314 to respective dispensing channels 222, it is possible to customize the configuration of chip 313 in accordance with the parameters to be determined for a given test. The detection reservoirs 223 in accordance with the expected clinical test items are provided in advance, a selective number of dispensing channels 222 are opened such that a sample can reach the detection reservoirs 223 which will give reactions responsible for the determination of the required items. On the other hand, according to this arrangement, the sample is prevented from entering unnecessary detection reservoirs 223. Thus, it is possible to provide a sufficient amount of sample to each detection reaction required for the test while consuming a minimum amount of sample.

Preparation of a chip as depicted in FIGS. 2 and 3 may be carried out, for example, via the procedures as described below. Grooves are formed on one surface of a substrate 216 to provide a main channel 221 and dispensing channels 222. An inlet 217 in communication with the main channel 221 is also formed together with detection reservoirs 223 and a reservoir 224. Formation of those channels and reservoir may be achieved by any appropriate means in accordance with the material of substrate 216, for example, when the substrate 216 is made of a plastic material, the grooves and others may be formed by etching, press molding using a die such as emboss molding, injection molding, molding using a material capable of curing via the exposure to light, and the like. The width of main channel 221 may be determined as appropriate in accordance with the property of a sample submitted to the test. For example, if a sample contains a polymer component (For example, DNA, RNA, protein, or saccharide chain), the main channel 221 preferably has a width of 5 μm to 1000 μm.

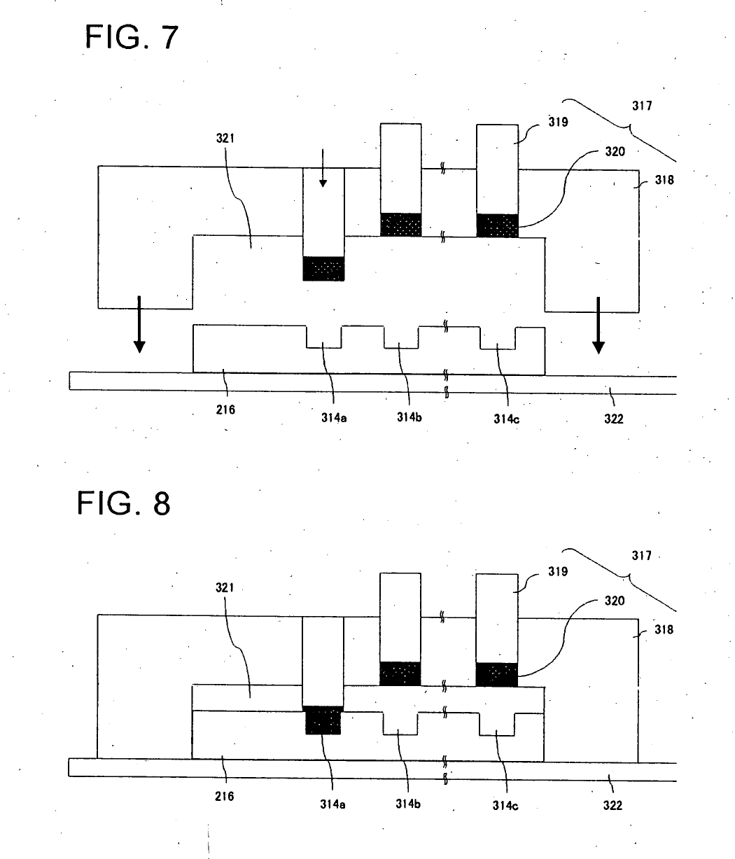

To each dispensing channel 222 is provided a flow control unit 314. The flow control portion 222 may have any desired structure as long as it can intercept the flow of liquid downstream through the dispensing channel 222 connected thereto as needed. For example, a flow control unit 314 may be formed by hydrophobizing a part of the dispensing channel 222. FIGS. 7 to 9 are the cross-sections of dispensing channels 222 for showing how the dispensing channel 222 can be selectively hydrophobic to intercept the flow of sample through the channel.

A substrate 216 shown in FIGS. 7 to 9 is placed on a platform 322. On the substrate 216 are provided three exemplary flow control units 314a to 314c as the flow control unit 314. In the particular example shown in FIGS. 7 to 9, the flow control unit 314a shall be closed while the flow control units 314b and 314c shall be kept open. Explanation will be given below how those flow control portions will see their assigned fates.

A flow control portion adjustment device 317 includes a pressing substrate 318 with a concave 321 having a size corresponding to that of a substrate 216, printing rods 319, and PDMS (polydimethyl siloxane) stamps 320. Printing rods 319 are provided opposite to respective flow control units 314, for example, with regard to the particular example shown in FIG. 7, to flow control units 314a to 314c. Each printing rod 319 has a PDMS stamp 320 on its tip, and is inserted into the pressing substrate 318 to be freely slidable therethrough up to downward or vice versa as shown in the figures.

Operation of the flow control portion adjustment device 317 proceeds as shown in FIG. 7 by projecting a printing rod 319 downward through the concave 321 towards a flow control unit 314 to be closed. In this particular example, since closure through flow control unit 314a is required, the printing rod 319 placed opposite to flow control unit 314a is projected to cavity 321 side.

FIG. 8 shows how the concave 321 is depressed until it engages with an underlying substrate 216 so that the flow control portion adjustment device 317 is pressed hard against the platform 322. The PDMS stamp 320 of the projecting printing rod 319 is deformed so much that the flow control unit 314a is filled therewith.

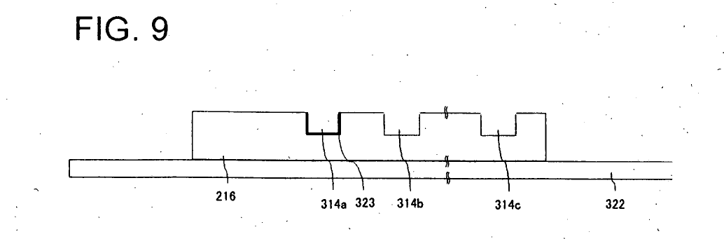

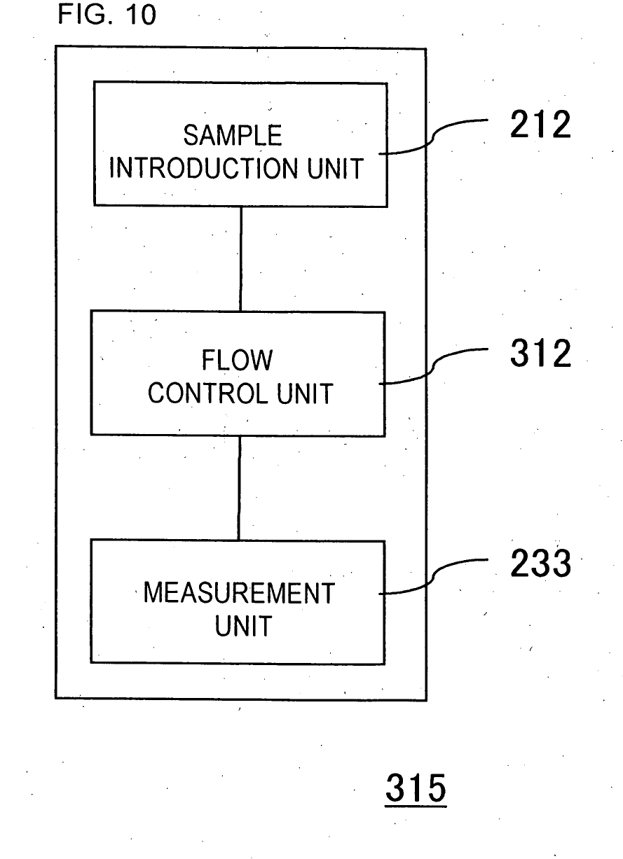

FIG. 9 shows the profile of flow control portions when the flow control portion adjustment device 317 is removed from platform 322. The flow control unit 314a has a thin layer 323 of PDMS formed on its surfaces as a result of the hard pressing of PDMS stamp 320 against it. Since the PDMS layer 323 is hydrophobic, sample flowing through the dispensing channel 222 connected to flow control unit 314a is prevented at that portion 314a from flowing further downstream. The band of hydrophobic PDMS layer 323 preferably has a width of 100 to 1000 μm.

According to this method, it is possible to reliably hydrophobize the surface of channels provided with flow control units 314 to be closed out of the flow control units 314 prepared on substrate 216 by bringing PDMS stamps 320 into contact with those flow control units 314. Thus, it is possible to close flow control units 314 to be closed easily, securely and selectively. Movement of printing rod 319 may be achieved, for example, by hand. Alternatively, a control unit for controlling the position of individual printing rods 319 may be provided to flow control adjustment device 317 so as to further facilitate the selective activation of printing rods 319. In this case, driving force necessary for moving selected printing rods 319 may be provided, for example, by a driving mechanism using a solenoid coil and magnet.

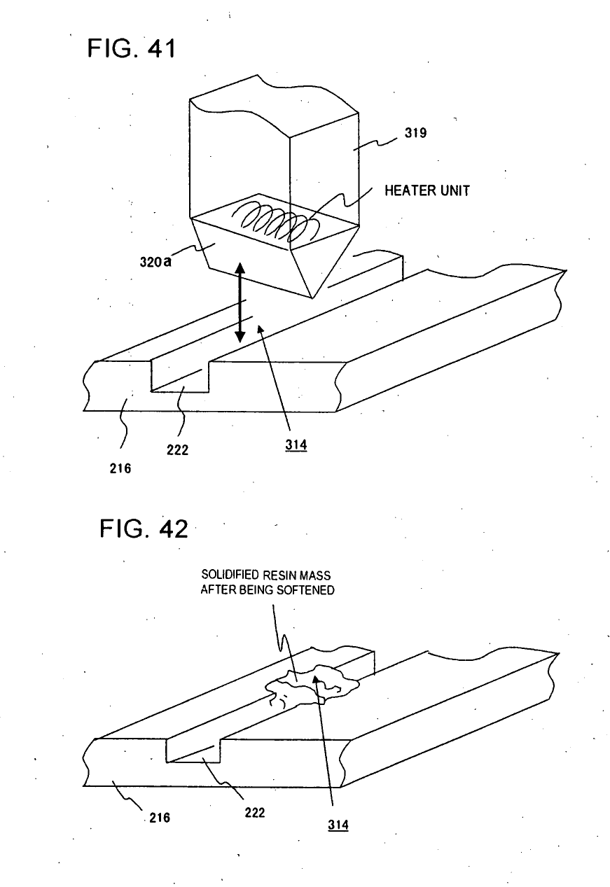

If the substrate 216 is made of a plastic material, it is possible to close a flow control unit 314 by using a printing rod with a heated stamp on its tip, and pressing the stamp against the flow control portion to cause a barrier to be formed there, thereby intercepting the channel. FIGS. 41 and 42 illustrate how a heated stamp is utilized to close a flow control unit 314.

As shown in FIG. 41, the stamp 320a attached to the tip end of printing rod 319 is heated to a temperature over the glass transition temperature of the constitutive material of substrate 216, and then pressed against a flow control unit 314 from the substrate 216 side. The stamp 320a may be made, for example, of a wedge-shaped metal strip protruding to the end. Heating of the stamp 320a may be achieved by a heater unit incorporated in the printing rod 319. The flow control unit 314 and its surrounds will soften via the contact with stamp 320a, and when the stamp 320a is further thrusted into the underlying substrate 216, the now softened resin constituting the substrate is pushed aside to form a bulge on the dispensing channel 222.

When the stamp 320a is removed from the substrate 216, and substrate 216 is cooled, the substrate 216 recovers its original hardness, and thus a barrier wall to seal and intercept dispensing channel 222 is formed as shown in FIG. 42.

According to this method, it is possible to readily seal the dispensing channel 222 and thus to intercept the flow reliably using a simple device. Then, it is possible to customize the configuration of a chip by providing the lay-out of the open/closure of individual flow control units 314. The metal strip to be used as stamp 320a may have its surface treated with Teflon (registered trademark). This arrangement will inhibit the adherence of plastic material of substrate 216 onto the surface of metal stamp 320a, which might otherwise occur when the metal stamp 320a is pressed hard against the substrate 216.

In the particular example shown in FIG. 42, the re-hardened resin bulge protrudes above the top surface of substrate 216, and such a bulge is preferably employed when the chip does not has a lid 226. If the bulge is not so high as to protrude above the top surface of substrate 216, removal of excess protrusion will be unnecessary even if the chip has a lid 226, which will make it possible to efficiently customize the configuration of analysis system in accordance with a given test.

According to the method underlying the embodiment, it is possible to readily customize the configuration of analysis system quickly in a short period of time because it is possible to opening/closing individual flow control units 314 via simple operation.

Turn again to FIGS. 2 and 3. An inlet 217 and vent holes 225 are formed on a lid 226.

The lid 226 is joined to the obtained substrate 216. Then, a seal 227 is applied as needed over the top surface of lid 226. Now, a chip 313 is obtained. Joining of lid 226 to substrate 216 may be achieved, for example, by apply a small amount of solvent to which the material of substrate 216 will dissolve onto the joining surfaces of substrate 216 and pressing the lid 226 against the surfaces of substrate 216. Alternatively, joining of lid 226 to substrate 216 may be achieved by applying a ultrasonic wave to the joining surfaces after the lid is attached to the substrate, or via a predetermined adhesive. If the lid 226 and substrate 216 are made of a plastic material, joining of the two may be achieved by welding.

The wall surfaces of main channel 221 and dispensing channels 222 is preferably coated in order to supress the adherence of molecules such as DNA or proteins thereto. Such coating will enhance the separation performance of a chip 313. Suitable materials for coating may include, for example, those that have a structure analogous to that of phospholipids constituting the cell membrane. It is also possible to supress the adherence of molecules such as DNA to the wall of channels, by coating the channel walls with a water-repellent resin such as fluorine contained resin, or with a hydrophilic substance such as bovine serum albumin. Or, a hydrophilic polymeric material such as MPC (2-methacryloyloxyethylphosphorylcholine) polymer or the like, or a hydrophilic silane coupling agent may be coated on the surface of a substrate 216.

If MPC polymer is used for rendering a surface of a substrate 216 hydrophilic, Lipidure (registered trademark, manufactured by NOF Corporation) or the like may be used. If Lipidure (registered trademark) is used, it may be dissolved in a buffer solution such as TBE (Trisborate+EDTA) buffer at 0.5 wt %, and the resulting solution is filled in main channel 221 and dispensing channels 222, and left for several minutes. Then, coating of the wall surfaces of those channels is achieved.

To further enhance the entry of a sample introduced via an inlet 217 into a channel 230, it is effective to form a hydrophilic layer such as silicon oxide layer on the surface of channel 230. It will be possible to smoothly introduce a buffer into a channel without the deliberate use of an external driving force, by forming a hydrophilic layer. Moreover, if at least the surface of the substrate 216 is made of a hydrophilic polymeric material such as PHEMA (polyhydroxyethylmethacrylate), it will be possible to prevent the non-specific adsorption of components contained in a sample to the surface of substrate 216. This will ensure the secure sorting and detection of a sample, even if the sample is very small in amount.

Turn back to FIG. 1. As described above, by using a chip 311 represented by the embodiment, it is possible to choose the detection of a predetermined component in a sample in accordance with the kind of the sample as appropriate by using a single chip 311. Thus, it is possible to carry out analysis only on the necessary parameters by using a minimum amount of sample.

Let's assume, for example, that in the chip 313 shown in FIG. 2, coloring reactions are allowed to occur at a plurality of the detection reservoirs 223. By so doing, it is possible to check whether any specific component is present in a sample or at which concentration the component is present in the sample by calorimetric method. In this case, the substrate 216 is preferably made of a transparent material. This is because such arrangement will ensure more accurate detection. Suitable materials may include, to mention concrete examples, quartz, cyclic polyolefin, PMMA (polymethylmethacrylate), PET (polyethyleneterephthalate), and the like.

Detection of a composition using a chip 313 is suitable for the detection directly using a sample introduced via an inlet 217. For this purpose, detection is preferably achieved at a single step in the detection reservoir 223. Suitable parameter for such detection may include, for example, the detection of alanine aminotransferase (ALT) which is a liver enzyme in blood plasma.

Incidentally, for the useless detection reservoirs 223 of a chip 313, that is, for the detection reservoirs 223 communicated with dispensing channels 222 whose flow control units 314 are closed, provision of detection reagents 231 may be omitted.

A chip 313 may be provided with another reservoir communicating with a main channel 221 so that the additional reservoir can contain buffer for diluting a sample or allow the introduction of buffer at a predetermined timing. By so doing it is possible to allow a sample to enter the system via inlet 217 to be diluted before it is guided to through dispensing channels 222 whose flow control units 314 are open as far as respective detection reservoirs 223. This arrangement makes it possible to dilute a sample to a concentration suitable for the occurrence of detection reaction at the detection reservoir 223, and thus to enable the highly sensitive measurement.

In the chip of this embodiment, it is possible to customize the configuration of the chip by selecting an opening/closing of flow control units 314, and thus the chip can be used profitably for performing the clinical laboratory test and the like. For example, a test item required for a hospital or a laboratory test center is easily selected, and it is to provide a chip suitable for the analysis of the test item. If the combination of the test item can be online-ordered by the hospital or laboratory test center, it will be possible for the laboratory to easily prepare necessary numbers of the made-to-order chips configured for the received test items.

It is also possible for a hospital or laboratory test center to readily prepare at site a chip suitable for the test item for a patient. With a view of managing his/her health, a user may make on-line access to a chip manufacturer and inform the manufacturer of his/her necessary health check items. Then, the manufacturer will be able to provide the user with necessary numbers of chips customized for the user's request.

Production of a customized chip will be detailed in relation to a ninth embodiment.

### Second Embodiment

This embodiment represents a chip that enables the selection of some parameters out of available plural parameters for the measurement by external measuring apparatuses. The chip includes plural measurement units responsible for the determination of respective detection parameters, which will also serve as analysis units. Each measurement unit is in communication with a dispensing channel to which is provided a flow control unit for adjusting the downward flow of liquid through the dispensing channel. The chip is configured such that a sample is guided only to the measurement unit responsible for the necessary parameters, by setting an opening and a closing of the flow control units connected to relevant measurement units.

FIG. 10 is a block diagram for representing the functional components of a chip representing this embodiment. A chip 315 is different from the one represented by the first embodiment in that it includes a measurement unit 233 instead of the detection unit 214. The measurement unit 233 is a portion for storing a sample component that will be subjected to the measurement by an external measuring apparatus.

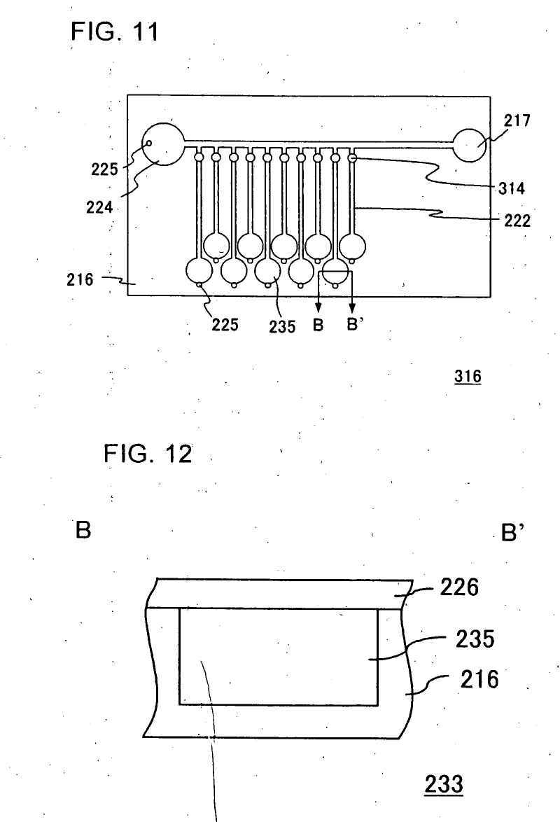

FIG. 11 shows the configuration of a chip 315 capable of achieving the functions as represented in FIG. 10. The basic structure of chip 316 shown in FIG. 11 is similar to that of chip 313 (FIG. 2) described in connection to the first embodiment, except that chip 316 includes fraction portions 235 instead of detection reservoirs 223. The fraction portion 235 is a reservoir for a fraction of a sample introduced via inlet 217.

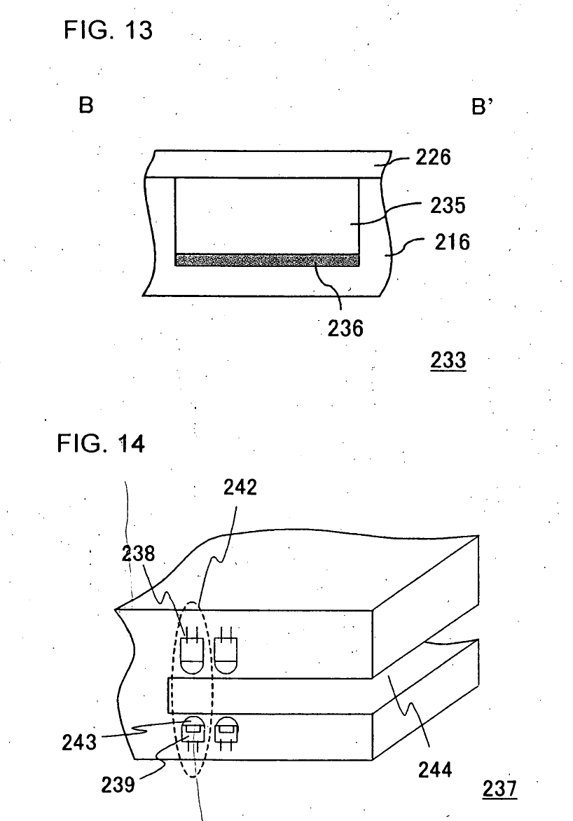

FIGS. 12 and 13 show the structure of measurement unit 233 that has a fraction portion 235 as a major structure member. The fraction portion 235 may be constituted only of a reservoir for storing sample as shown in FIG. 12. The separation portion 235 may further contain a measurement reagent 236 as shown in FIG. 13. The measurement reagent may include the same substances that are utilized as detection reagents 231 in a chip 313 representing the first embodiment. By using the measurement reagent 236, a specific element in sample is reliably analyzed by using a coloring reaction. To put it more specifically, it is possible to measure the intensity of light having a wavelength of 350 to 640 nm transmitted therethrough. Even when the measurement portion does not contain any measurement reagent like the one shown in FIG. 12, it is possible to select number of the fraction portions 235 used by setting the opening and closing of the flow control units 314 to meet the need of estimation for the bias value due to the coloration of the sample in itself.

FIG. 14 is a diagram for schematically showing the structure of a measuring apparatus 237 which will perform an optical measurement on a sample component in the fraction portions 235, by receiving the insertion of chip 316. The measuring apparatus 237 includes a socket 244 for receiving the insertion of a chip 316 and a measuring unit 242 for measuring the optical property of a sample in a fraction portion 235 of a chip 316 inserted into the socket 244 by irradiating a light thereto. The measuring unit 242 includes a light source 238, a light condenser 243, and photosensitive portion 239. In FIGS. 14 to 16, only two measurement units 242 and two fraction portions 235 are depicted for the convenience of illustration, but the measurement units 242 may be provided by the same number with that of the measurement units 233 provided on a chip 316.

The size of measurement unit 242 may be determined in accordance with the size of fraction portion 235 related thereto. For example, it is possible to prepare a chip 316 where each fraction portion 235 has a depth of about 1 mm, and there is an interval of about 1 mm between any two adjacent fraction portions 235. Then, each of the light source 238, the photosensitive portion 239 and the optical filter 240 is designed to have a size matching the dimension of fraction portions 235.

The light sources 238 may include, for example, an LED, laser diode, semiconductor laser, and so on. The action of light source varies depending on the wavelength for measurement, and thus the light source may be chosen as appropriate according to the wavelength of light emitted as a result of the coloring reaction based on a measurement reagent 236 under study. The light condenser 243 may be obtained, for example, by processing a self-focusing lens into a lens having a desired shape and size. Suitable photosensitive portion 239 may include a phototransistor, photoelectric cell, and so on.

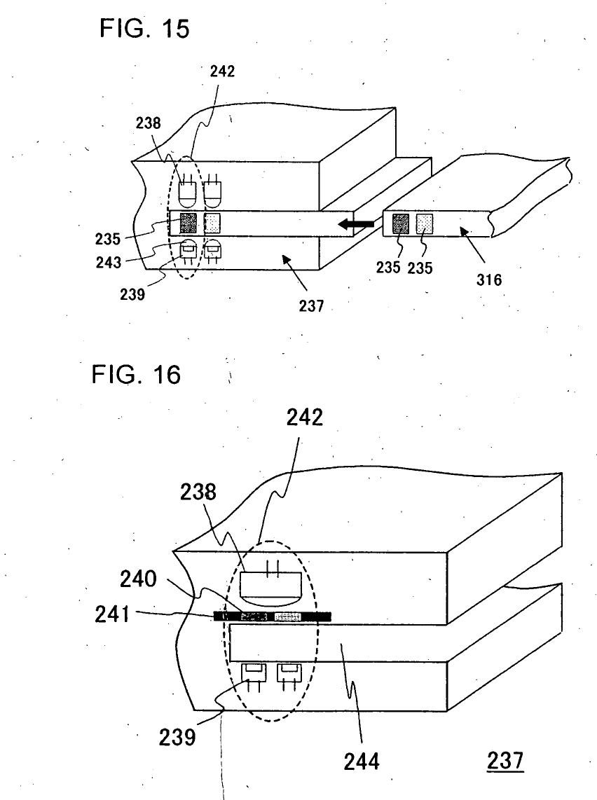

FIG. 15 illustrates how a chip 316 is inserted into the measuring apparatus 237 shown in FIG. 14. When a chip 316 is inserted into the socket 244 of a measuring apparatus 237, opposite to a measuring unit 242 is placed a corresponding fraction portion 235. To satisfy this condition, the measuring apparatus has the measurement units 242 of the same in number with that of fraction portions 235 of a chip 316 it receives. Then, the fraction portions 235 of chip 316 will be placed opposite to their corresponding measurement units 242, thereby enabling optical measurements to be performed on all the separation portions simultaneously, which will allow the measurement to be completed quickly. Alternatively, the measuring apparatus 237 may have a single measuring unit 242. Then, it will be possible to achieve optical measurement by sliding the chip 316 through the socket 244 so that a plurality of the fraction portion 235 is sequentially measured.

FIG. 16 shows a variation of the measuring apparatus 237. The measuring apparatus 237 shown in FIG. 16 is similar in its basic structure to the apparatus shown in FIG. 14, but different in that it includes a single light source 238, and further includes an optical filter 240 and light shield plate 241. The measurement unit shown in FIG. 16 lacks a light condenser 243, but may have one.

Using the optical filter 240, a light from the light source 238 and whose wavelength falls in a specified range can selectively be irradiate with to the fraction portion 235. Because of this arrangement, it is possible, even when the light source 238 emits light consisting of components having a wide range of wavelengths, to select the components having wavelengths falling within a specified range using the optical filter 240 in accordance with the wavelength for a measurement, thereby using only those light components for the measurement. In addition, since the optical filter 240 is supported by a light shielding plate 241, leak of a light from the measurement unit 242 irradiated from light source 238 to adjacent units 242 can be safely avoided.

The optical filter 240 may be obtained by cutting a material known as a material for optical filter to a specified size.

For the measurement apparatus 237 as shown in FIGS. 14 to 16, instead of the fixed light source 238, an optical fiber from a remote light source may be placed with respect to the measurement apparatus 237 such that it directs light from the light source to an assigned fraction portion 235. The above explanation has been given on the premise that light from a light source transmits the fraction portion 235. However, the measurement unit 242 may be constructed so as to monitor the absorption or scattering of light.

The configuration of chip 315 shown in FIG. 10 is not limited to those described above, but may take many other variations. For a chip 316 where measurement units 233 is served as analysis units, it is possible to customize the configuration of the chip 316 in accordance with the parameters required for a given test by providing flow control units 314 to respective dispensing channels 222 communicating with the fraction portions 235 and opening/closing the flow control channels 314. The chip 316 is allowed to have in advance fraction portions 235 prepared in accordance with the expected test items. Then, it is possible to allow a sample to flow through the dispensing channels 222 connected to the fraction portions 235 which are involved in the determination of necessary parameters while the sample is prevented from entering unnecessary fraction portions 235. Thus, it is possible to provide a sufficient amount of sample required for the test while consuming a minimum amount of sample. Therefore, it is possible by using the measurement apparatus 237 to reliably perform necessary tests about the components of a sample via simplified procedures.

The configuration of chip 316 embodying the essence of the chip 315 shown in FIG. 10 and that of the measurement apparatus 237 are not limited to those described above, but may include many variations.

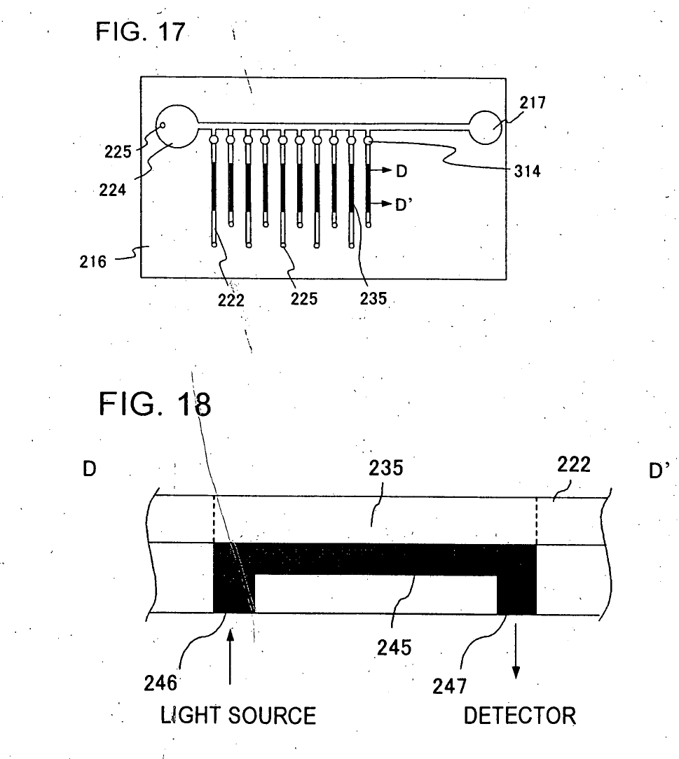

For example, the chip may include a variation as shown in FIG. 17 where each fraction portion 235 is placed over the corresponding dispensing channel 222, and an optical waveguide 245 is placed beneath the fraction portion 235. The optical waveguide 245 may be made of a quartz-based material or an organic polymer-based material. The optical waveguide 245 is made of a material having a higher refractive index than that of surrounding materials. In this case, light enters from the bottom of the chip into the optical waveguide 245, and exits outside from the bottom of the chip. FIG. 18 shows a cross-section of the chip shown in FIG. 17 cut along line D-D′.

In this case, a light source may be provided on the bottom surface and the like of measuring apparatus 237 so that it directs light towards an optical waveguide for illumination 246, while a photosensitive portion may be provided on the bottom surface and the like of measuring apparatus 237 to receive light emanating from an optical waveguide for sensor 247. Then, the surface in which the optical waveguide for illumination 246 and the optical waveguide for sensor 247 are exposed may attached to the bottom surface and the like of measuring apparatus 237, so as to guide the entry of light into the fraction portion 235 of a dispensing channel 222 and to receive light emanating from the fraction portion 235 for measurement.

The chip shown in FIGS. 17 and 18 may be constructed so as to have the optical waveguide for illumination 246 and the optical waveguide for sensor 247 while being devoid of the optical waveguide 245. With the chip of this configuration, light emanating from a light source is guided via the optical waveguide for illumination 246 to a fraction portion 235 which passes the light via the optical waveguide for sensor 247 to a photosensitive portion. With the chip of the above configuration, it is possible to perform optical measurement on a specified component in a liquid captured in the fraction portion 235. Since the chip lacks the optical waveguide 245, it is possible to simplify the structure of the chip.

The above explanation has been given on the premise that the measuring apparatus 237 uses, for detection, light transmitted through a fraction portion 235. However, the photosensitive portion 239 may be constructed so as to use, for detection, light reflected from a fraction portion 235.

Instead of being applied to the measuring apparatus 237 for measurement, the chip 316 may be constructed so that an aliquot can be dispensed from sample captured in a fraction portion 235 of the chip 316 to be measured by an external measuring apparatus.

It is possible to detect the presence, for example, of ALT that is one of the hepatic enzyme at the fraction portion 235 of chip 316. For example, if a sample consisting of blood plasma is introduced into inlet 217, it, will be guided only to the fraction portions 235 whose flow control channels 314 are kept open. If one of those fraction portions 235 whose flow control channels 314 are kept open is allowed to contain in advance a measurement reagent 236 comprising, for example, L-alanine, α-ketoglutaric acid, β-reduced nicotinamideadeninedinucleotide (NADH), L-lactatedehydrogenase (LDH), and so on, a coloring reaction represented by:
NADH—>NAD⁺
will occur at the fraction portion 235 which will be detected by the measuring apparatus 237. The ALT activity is determined based on the altered transmittance of light having a wavelength of 340 nm passing through the fraction portion 235 and detected by the measuring apparatus 237. To eliminate the contribution of non-specific absorption, dual wavelength measurement including the measurement at the wavelength of 405 nm may be performed.

### Third Embodiment

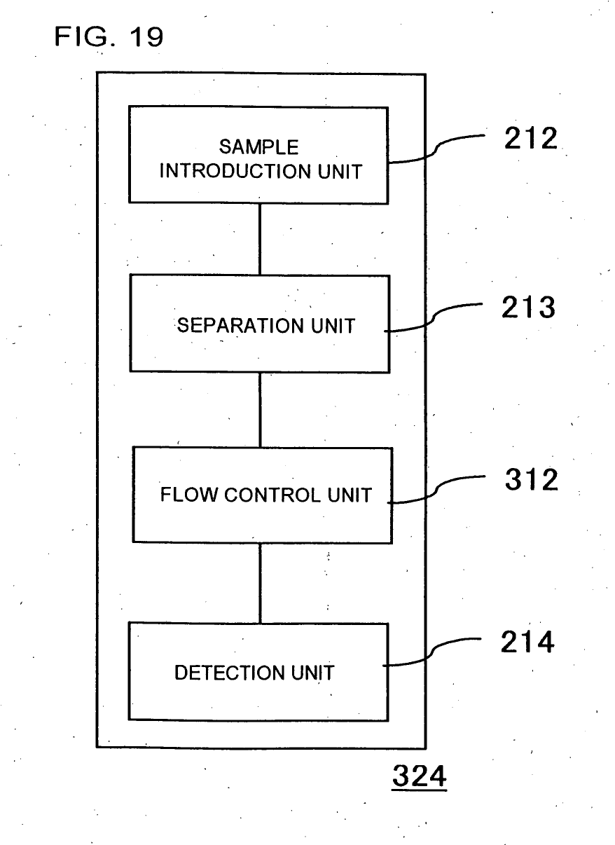

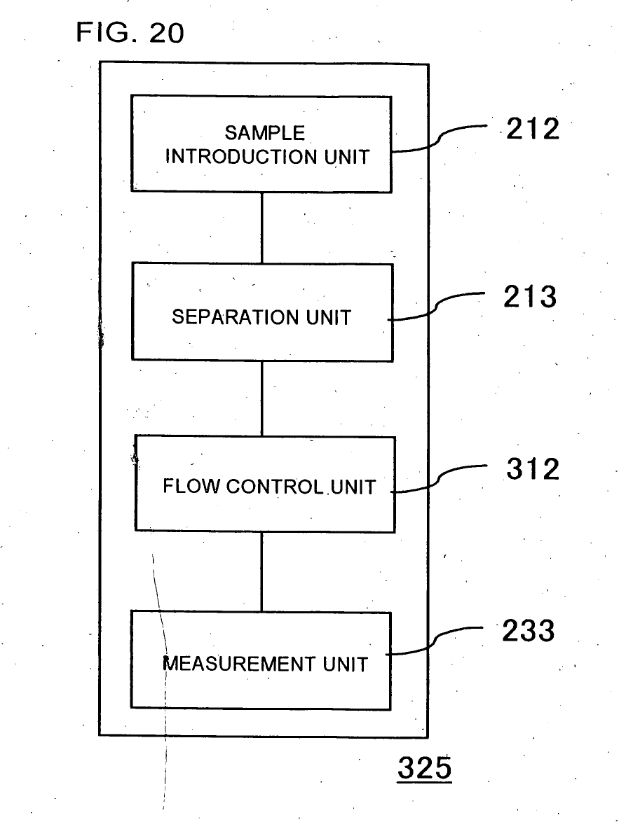

The chip described in relation to the first and second embodiments may further include a separation unit for separating specified components of a sample before the sample is subjected to analysis (detection or measurement), at an intermediate stage between the sample sample introduction unit 212 and the flow control unit 312. FIGS. 19 and 20 are block diagrams for representing the functional components of chips representing this embodiment of the invention. The chips 324 and 325 shown in FIGS. 19 and 20 respectively further include a separation unit 213 between sample introduction unit 212 and flow control unit 312 and makes it possible to perform analysis (detection or measurement) on the component separated in advance from a sample. Description will be given below taking as an example a chip including a detection unit 214, which serves as an analysis unit (FIG. 19).

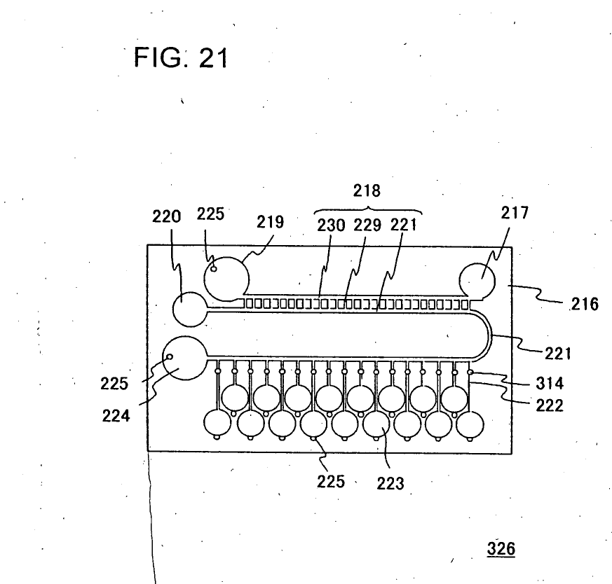

FIG. 21 is a diagram for representing the components of an exemplary chip further including a separation unit 213. The chip 326 shown in FIG. 21 is similar in its basic structure to the chip 313 shown in FIG. 2 except that it includes between sample inlet 217 and dispensing channels 222 a separation region 218, which contains part of main channel 221. The chip 326 further includes, in addition to the components of the chip shown in FIG. 2, a waste reservoir 219, a buffer inlet 220, and a channel 230. The number of detection reservoirs 223 may be determined as appropriate.

The separation region 218 includes the channel 230, main channel 221, and plural thin channels 229 connecting them, and acts like a filter. In communication with the channel 230 there is provided the waste reservoir 219 for the disposal of waste. In communication with the main channel 221 there is provided the buffer inlet 220. With the exemplary chip 326 shown in FIG. 21, the separation region 218 acts like a filter. However, the operation style of separation region 218 is not limited to the above, but may include many variations.

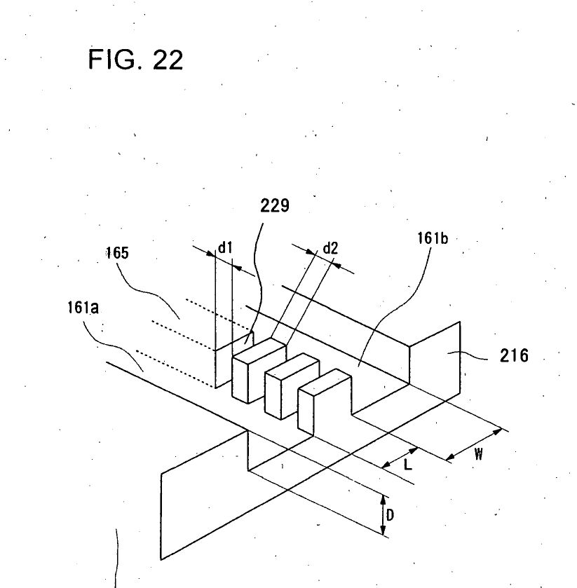

FIG. 22 illustrates the structure of separation region 218. Referring to FIG. 22, there are formed on a substrate 216 channel grooves 161a and 161b (each having a width W and depth D) with a partition wall 165 in between. Either one of the channel grooves 161a and 161b serves as main channel 221 and the other as channel 230. Across the partition wall 165 there are formed separation channels at regular intervals. The term “separation channel” used herein represents each of the thin channels 229. The separation channels each having a width of d1 cross at right angles with channel grooves 161a and 161b, and are arranged with a constant interval of d2 between adjacent separation channels. The dimensions of the components shown in the figure may be altered as appropriate depending on the type of samples to be separated, and, for example, appropriate values may be chosen from the ranges cited below.

W: 10 μm to 1000 μm

L: 10 μm to 1000 μm

D: 50 nm to 1000 μm

d1: 10 nm to 10 μm

d2: 10 nm to 100 μm

Out of the above dimensions, the value L defining the length of separation channel must be precisely determined according to the analysis purpose because it directly affects the separation ability. For example, if separation of a polymer is required, the polymer will undergo conformational change during its passage through a separation channel that will cause the change of enthalpy. The overall change of enthalpy a polymer undergoes during its passage through a separation channel varies depending on the length of the separation channel. Thus, from the viewpoint of separation channel, its length affects the separation ability of the separation channel. In the invention, since the channels consist of grooves, it is possible to form them by etching or molding while precisely controlling their shape and size. As a result it is possible to stably produce a separation device having a specified separation ability. Formation of channel grooves 161a and 161b, and separation channels may be achieved by any appropriate method, but is preferably practiced by dry etching in combination with electron beam exposure, particular when the separation channel is designed to have a depth d1 and interval d2 of 100 nm or less.

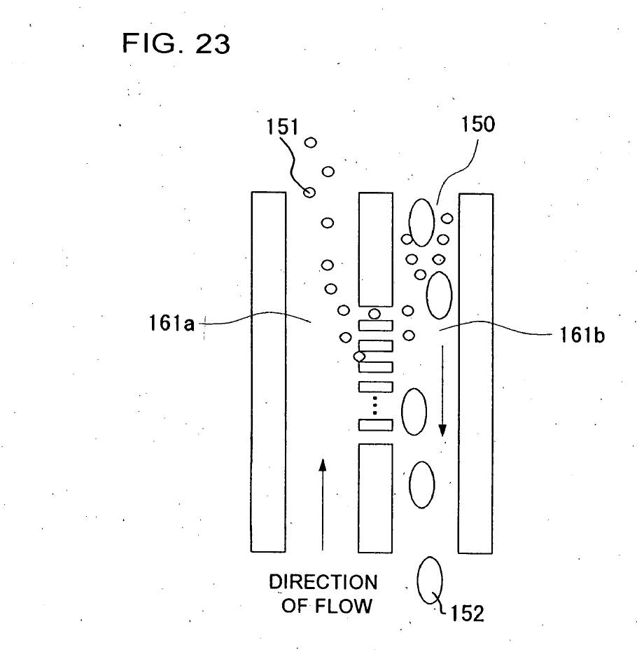

Separation of molecules occurring at the separation region 218 shown in FIG. 22 will be described with reference to FIG. 23. FIG. 23 is a top view of the separation device for showing the outline of the structure thereof. First, before separating components from a sample, a buffer serving as a carrier is allowed to flow through the channel grooves. Referring to FIG. 23, an original sample containing a mixture 150 of different components flows from up downward in the figure through channel groove 161b. Then, smaller molecules 151 of the mixture pass through separation channels provided to the partition shown at the center of the figure and enter into the adjacent channel groove 161a. Through the channel groove 161a there flows from down upward in the figure a solvent that is chosen so as not to react with the molecules to be separated. Hence, the smaller molecules 151 entering the channel groove 161a move upward in the figure being carried by the flow of solvent. On the other hand, larger molecules 152 flowing along channel groove 161b cannot pass through the separation channels, and keep flowing in the same direction along the channel groove 161b. Thus, smaller molecules 151 are separated from larger molecules 152.

In the separation unit shown in FIG. 22, currents flowing along the channel grooves 161a and 161b are opposite to each other. But, the two currents may flow in the same direction. Use of two currents flowing in the opposite directions, however, will improve the separation efficiency of the unit. For example, the current flowing along channel groove 161a may be allowed to flow from up downward instead of from down upward. Then, a concentration of smaller molecules 151 becomes higher with its passage through the separation path. Thus, the concentration of smaller molecules 151 in the current flowing along channel groove 161a approaches the counterpart in the channel groove 161b until the former equals to the latter at certain point. Ahead of the point, migration of larger molecules 152 through separation channels will rarely occur, thus incapacitating the separation of larger molecules 152. In contrast, when the two currents flow in the opposite directions as in the present embodiment, the difference in concentration of larger molecules 152 between the two currents flowing along channel grooves 161a and 161b is maintained, and thus the high separation ability of the unit is ensured even when the separation channel has a certain large length.

In the above embodiment, plural thin channels 229 which serve as separation channels are formed through the partition wall. The separation region 218 may include a bank portion as in an embodiment described below.

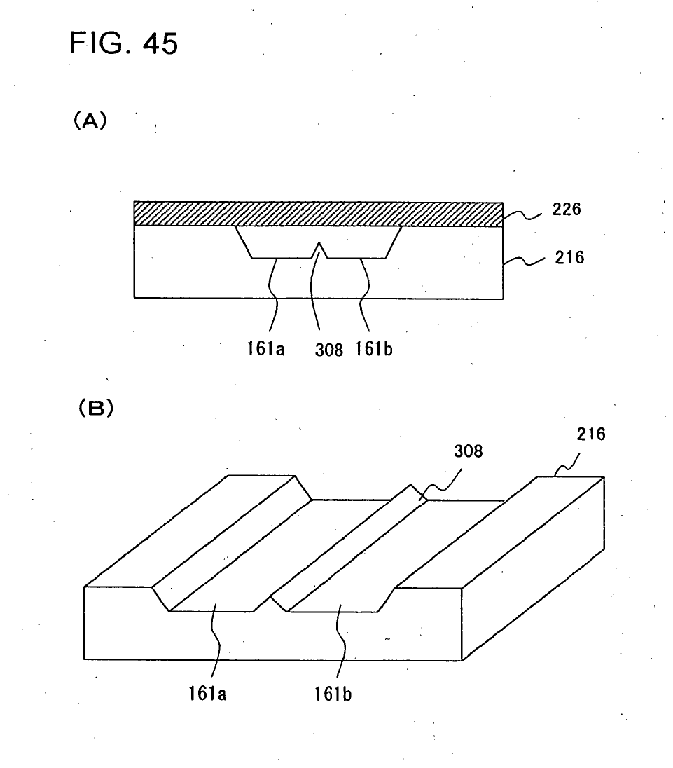

FIG. 45 shows another configuration of the separation region 218. separated Figs. A and B show its sectional and perspective views respectively. As shown in FIG. 45A, on a substrate 216 there are formed two parallel channel grooves 161a and 161b with a partition wall 308 between the two grooves. The partition wall 308 is in the form of a bank. A lid 226 is placed over the substrate 166. In FIG. 45B, the lid 226 is not represented for the convenience of illustration.

As seen from FIG. 45A, a blank space exists between the summit of partition wall 308 and the lid 226, and thus the two channel grooves 161a and 161b communicate with each other through the blank space. This blank space corresponds to the separation channels formed through the partition wall 165 of separation region 218 described above. Thus, it is possible to practice separation operation by flowing a sample containing a substance to be separated along channel groove 161a, and a buffer along channel groove 161b.

In this case, the lid 226 is preferably made of a hydrophobic material such as polydimethylsiloxane, polycarbonate and so on. By so doing it becomes possible to reliably introduce sample and buffer into their respective channel grooves without taking the risk of cross-contamination between the two, and to allow the two solutions to contact with each other via the blank space at a time when the two channel grooves are filled with sample and buffer. The same advantage will be ensured by lacking the lid 226, because then air will act as a hydrophobic substance, and perform the same task as does the lid 226.

Let's assume that the separation unit has a lid 226 made of a hydrophilic material such as polyethylene terephthalate. If a sample is passed through, for example, channel groove 161a, part of the sample will move to the other channel groove 161b. During the movement of sample, only the molecules whose size is smaller than the blank space formed between the partition wall 308 and the lid 226 will be allowed to selectively pass through the space. Thus, separation of smaller molecules can be achieved.

According to this embodiment, by providing the partition wall 308, since one current flowing along channel groove 161a and the other current flowing along channel groove 161a allowed to contact with other, separation of target molecules occur at a larger extent than is possible with the above embodiment where separation of target molecules occur at thin separation passages 229 formed at a part of partition wall 165, which will improve the efficiency of separation. If the molecules have a slender contour, they will easily pass through the space without being captured there. Thus, the unit can be profitably used for the analysis of a sample that contains molecules having such a complex shape.

Formation of channel grooves 161a and 161b with a partition wall 308 in between may be achieved by processing a (100) Si substrate using wet etching. When a (100) Si substrate is used, a groove with a trapezoidal profile as shown in the figure will be formed by etching in a direction in parallel with or normal to (001) direction. It will be thus possible to determine the height of partition wall 308 by adjusting the etching time.

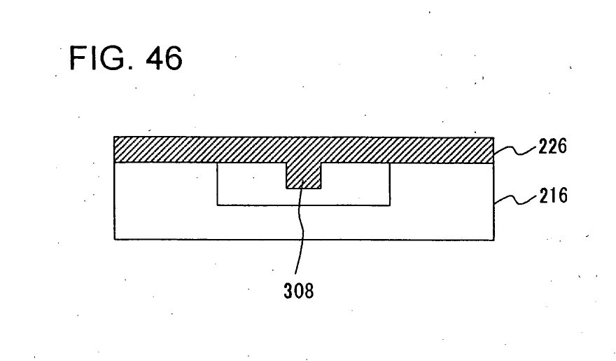

The partition wall 308 may be formed on the lid 226 as shown in FIG. 46. It is possible to readily obtain a lid 226 with a partition wall 308 by subjecting a resin such as polystyrene to injection molding. On the other hand, it is possible to obtain the substrate 216 by forming a single passage groove on a substrate 216 by etching. Since it is possible to process the separation region 218 by simple procedures as described above, the chip is suitable for mass production.

As seen from above, by attaching a separation region 218 to part of main channel 221, it is possible to separate the introduction of liquid sample into the system via capillary action and the separation of target components via diffusion. Separation of target molecules may occur via their difference in osmotic pressure.

Turn back again to FIG. 21. A sample, after being introduced into inlet 217, is guided via capillary action to channel 230. When the channel 230 is filled with the sample, a predetermined buffer is introduced via the buffer inlet 220. The buffer is used as a mobile phase for separating components contained in the sample. Buffer, after being introduced into buffer inlet 220, is guided via capillary action to main channel 221, and flows in a direction opposite to the direction in which sample in the channel 230 flows.

Since thin channels 229 connecting channel 230 and main channel 221 have a smaller width or depth than the channel 230, in the channel 230 only the molecules having predetermined size and shape can pass through the thin passages 229 to reach the main channel 221 to merge the current there. The molecules which cannot enter the thin channels 229 are evacuated into the waste reservoir 219. Thus, the molecules of a sample can be separated according to a size or a shape they take in the mobile phase. The thin channels 229 may be substituted for tiny orifices formed on a thin partition separating channel 230 and main channel 221.

Introduction of such a separation region 218 will make it possible to subject a sample to coarse separation or refined purification. By subjecting a sample to coarse separation, it is possible to remove the sample of solid objects, cells and so on. If a sample is liquid, it is possible to separate low molecular weight components from high molecular weight components.

Sample components flowing through the main channel 221 are distributed to the dispensing channels 222 communicating with the main channel 221 to be guided to the respective detection reservoirs 223 for dispensing. For the chip 326 as in the chip 313 shown in FIG. 2, the sample is distributed only to the detection reservoirs 223 which are connected to the dispensing channels 222 whose flow control units 314 are kept open.

In the embodiment, since flow control units 314 of dispensing channels 222 with their detection reservoirs 223 are provided downstream of separation region 218, it is possible to perform detection or measurement operation on the component having undergone separation operation secondary to the introduction of sample via inlet 217, in accordance with the parameters required for a given test. Thus, it is possible to customize the configuration of the chip 326 by setting an opening and closing the flow control channels 314 connected to respective dispensing channels 222. According to the embodiment, since target components in a sample are separated in advance prior to analysis, it is possible to perform high sensitivity detection operation at the detection reservoir 223.

For example, it is possible to determine the concentration of sugar in blood at a detection reservoir 223 of a chip 326. In this case, when a blood sample is introduced via an inlet 217, red cells are separated out at the separation region 218. The plasma component is then diluted with buffer introduced via a buffer inlet 220 and guided to the detection reservoir 223. A detection reagent 231 including NAD (β-oxidized nicotinamide adenine dinucleotide), ATP (adenosine triphosphate sodium), hexokinase, glucose-6-phosphatedehydrogenase, and magnesium acetate is put in advance in the detection reservoir 223. The coloring reaction occurs at the detection reservoir 223. Then, the blood sugar level of the sample can be easily determined by measuring the intensity of coloration.

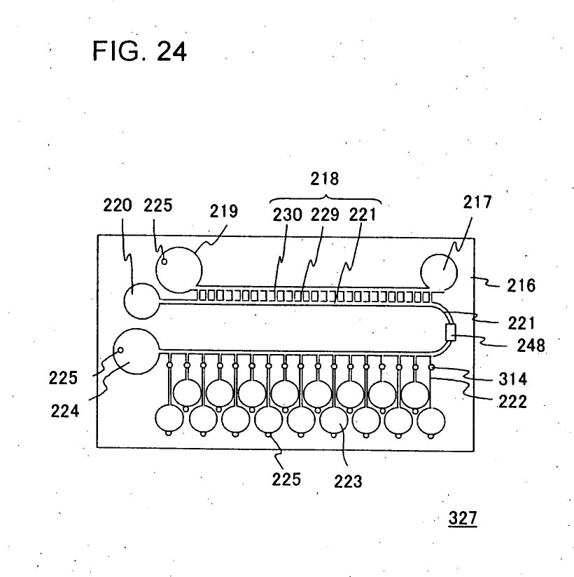

The present embodiment may further include a mixing portion between the separation unit 213 and analysis unit (detection unit 214 or measurement unit 233) to enhance the homogenous mixture of sample before the sample is subjected to detection or measurement. Explanation will be given below with reference to a chip including a detection unit 214. FIG. 24 shows the configuration of an exemplary chip having a mixing portion 248. The chip 327 shown in FIG. 24 is similar in its basic structure to the chip 326 shown in FIG. 21 except that it includes a mixing portion 248 at a section of main channel 221 between separation region 218 and dispensing channels 222.

The mixing portion 248 of the chip 327 is not limited to any specific shape and structure, as long as it can homogenize the sample flowing through the main channel 221, but may take, for example, the following structure.

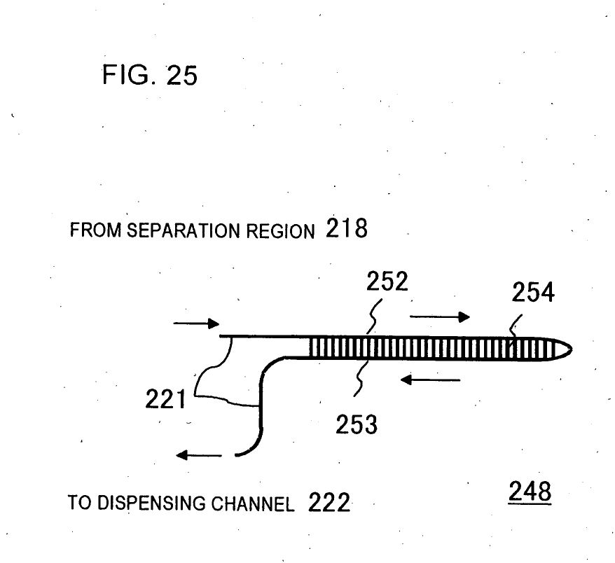

FIG. 25 shows an exemplary configuration of the mixing portion 248. The mixing portion 248 shown in FIG. 25 consists of an entrance passage including a current and counter current to enhance the vigorous agitation of flow therethrough. The entrance passage includes a forward passage 252 and backward passage 253 both forming the segments of main channel 221, and tiny passages for mixing 254 connecting the two passages. The tiny passages for mixing 254 may be substituted, for example, for tiny orifices formed on a partition separating the forward passage 252 from the backward passage 253.

The surfaces of fine channel for mixing 254 are preferably more hydrophobic than those of forward passage 252, because then current will not move from forward passage 252 through the fine channel for mixing 254 to backward passage 253 until the forward passage 252 is filled with liquid flowing from the separation region 218. When the forward passage 252 is filled with the liquid, and the liquid reaches the head portion of backward passage 253, into the fine channel for mixing 254 one fraction of liquid enters from backward passage 253 and another fraction of liquid enters from forward passage 252, and the two fractions of liquid collide in the fine channel for mixing 254 where dispersion of elements to opposite liquid masses occurs which contributes to the homogenization of liquid. The thus homogenized liquid flows along main channel 221 to be distributed via dispensing channels 222 to their respective detection reservoirs 223.

Thus, it is possible to homogenize the concentration of a liquid reaching dispensing channels 222 after having passed the backward passage 253. Accordingly, even if the density of sample components contained in the liquid having passed the separation region 218 is uneven, it is possible to provide fractional liquids where the density of a sample component is homogenized to selected detection reservoirs 223, which will elevate the precision of detection of reaction occurring at the detection well.

Let's assume, for example, that a front part of a liquid mass has a higher density of sample components. As the liquid mass moves from main channel 221 to forward passage 252, its front part intermingles with the inflow of liquid from backward passage 253 which has been sufficiently mixed to have a comparatively homogeneous density, and thus the front part comes to have a more even density of components with progression of the liquid mass through the mixing portion. Then, let's assume the contrary case in which a tail part of a liquid mass has a higher density of sample components. The liquid mass moves from main channel 221 to forward passage 252. It occurs at a certain time that when the front part of liquid mass runs backward passage 253, its tail part moves still along forward passage 252. Then, its front part having a lower density of components intermingles with the inflow of liquid from the tail part having a higher density of components, which will homogenize the density of the two parts in question. In the particular embodiment shown in FIG. 25, the main channel 221 is in the form of a straight line, but it may take a zigzag line or spiral line. By so doing it is possible to further miniaturize the mixing portion 248, thereby reducing the overall size of a chip.

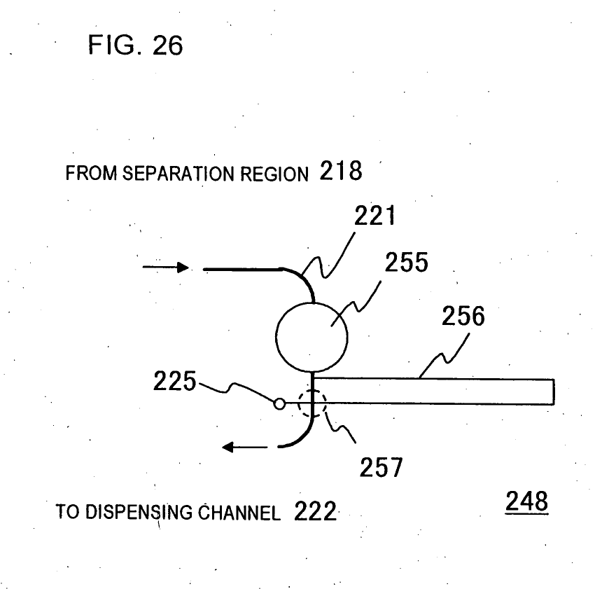

FIG. 26 shows another configuration of the mixing portion 248. The mixing portion 248 shown in FIG. 26 includes a reservoir 255 along the path of main channel 221, and, downstream of the reservoir 225, a trigger channel 256 connecting main channel 221 at its two different sites. The trigger channel 256 is a collateral channel connecting two different sites of main channel downstream of the reservoir 255. It is possible to control the flow velocity of a liquid through the trigger channel 256 by changing the hydrophilic property or the size of the channel as appropriate. This helps to control the speed of switching operation. The trigger channel 256 has two intersections with main channel 221. The intersection on the downstream side or towards dispensing channels 222 includes a liquid switch portion 257.

With the mixing portion 248 configured as described above, the liquid switch portion 257 is initially closed, and thus liquid from the separation region 218 accumulates in the reservoir 255, and the conentration of the liquid is homogenized. When the reservoir 255 is filled with liquid, a fraction of the liquid enters into the trigger channel 256. When the trigger channel 256 is filled with liquid, and the front part of liquid reaches the liquid switch portion 257, the liquid switch portion 257 is opened, and liquid whose cocentration is homogenized in the reservoir 255 is allowed to flow towards dispensing channels 222.

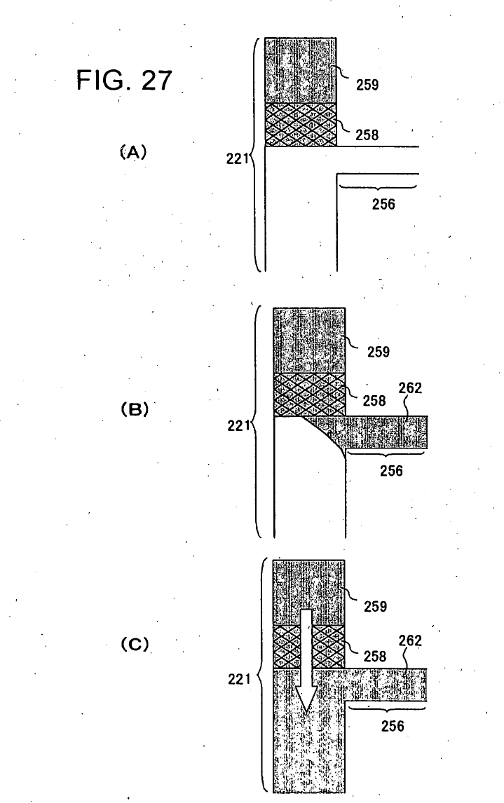

FIGS. 27A to 27C are top views of the liquid switch portion 257 shown in FIG. 26. The liquid switch portion 257 is a switch for controlling the movement of liquid, and is opened/closed via the triggering action of liquid movement. FIG. 27A depicts the switch closed; and FIGS. 27B and 27C the switch opened. In the figure, to the side surface of main channel 221 is connected the trigger channel 256. It is possible to control the flow velocity of liquid through the trigger channel 256 by changing the hydrophilic property or the diameter of the passage as appropriate. This helps to control the speed of switching operation. Upstream (upward in the figure) of the intersection between main channel 221 and trigger channel 256, there is provided a damming portion 258. The damming portion 258 is so constructed as to exert stronger capillary action than other adjacent portion in the channel. As exemplary structures of the damming portion 258, followings may be mentioned.

(i) Structure Constituted of Plural Columnar Bodies

The damming portion 258 having this structure has a larger surface area per unit volume than other channels corresponding in size. Therefore, even when the main channel 221 is filled with liquid, the damming portion 258 will have a larger solid-liquid interface than other parts of the channel.

(ii) Structure Filled with Porous Particles or Beads

The damming portion 258 having this structure will have a larger solid-liquid interface than other part of the channel.

(iii) Structure Consisting of Hydrophobic Surfaces

The damming portion 258 having this structure will intercept the passage of liquid because of the lyophobic activity of its columnar walls.

When the damming portion 258 adopts structure (i), the dimension of the columnar body may be determined according to the shape and material of a substrate. If a substrate made of glass or quartz is used, the columnar bodies may be prepared by photo-lithography or dry etching. If a substrate made of plastic is used, a die is prepared that includes a negative cast of the columnar bodies, and the desired columnar bodies are obtained by molding plastic with the die. Incidentally, such a die as described above can be obtained by photo-lithography or dry etching.

When the damming portion 258 adopts structure (ii), that damming portion will be obtained by filling the necessary part of main channel with porous particles or beads, or attaching porous particles or beads by adhering to the walls of the necessary part of main channel.

The present embodiment will be described on the premise that the damming portion thereof adopts structure (i).

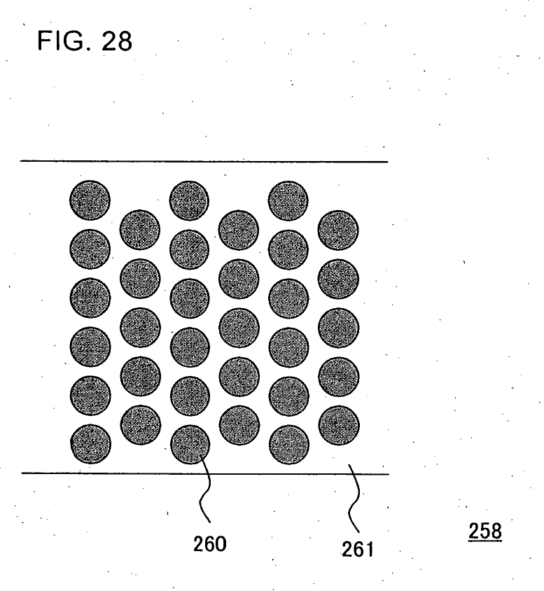

FIG. 28 is a top view of the damming portion 258. Plural columnar bodies 260 are arranged in an orderly fashion with a constant interval between adjacent columnar bodies. The space left by the columnar bodies 260 constitutes a fine channel 261. The damming portion 258 having this structure has a larger surface area per unit volume than other chanels corresponding in size. Because of this, liquid reaching the damming portion 258 is prevented from advancing further by the capillary action exerted by the damming portion, and thus it is holded at the fine channel 261.

FIG. 27A shows the stand-by state of liquid switch portion 257. At the state, liquid sample 259 having flown along main channel 221 is held at the damming portion 258. At a desired timing under this state, when a triggering liquid 262 is allowed to pass through the trigger channel 256, as shown in FIG. 27B, the front part of liquid mass passing through the triggering liquid 262 comes into contact with the damming portion 258. Although, under the state shown in FIG. 27A, the liquid sample 259 is held stationary on account of the capillary action exerted by the damming portion 258, at the moment when the triggering liquid 262 comes into contact with the liquid sample 259 as shown in FIG. 27B, the liquid sample 259 moves downstream (downward in the figure) to resume its flow. Namely, the triggering liquid 262 serves as priming water and the function as a liquid switch portion is achieved for urging the downward flow of liquid sample 259.

Both the liquid sample 259 and triggering liquid 262 have passed reservoir 255. Thus, according to this embodiment, it is possible to prohibit the entry of sample to dispensing channels 222 for the period elapsed while the sample has passed separation region 218, filled reservoir 255 and reached to the front part of the trigger channel 256, that is, the intersection of main channel 221 with trigger channel 256. Since, during the period, homogenization of the concentration by diffusion and the like in reservoir 255 is advanced, it is possible to further enhance the homogenization of the concentration of sample components.

The timing at which liquid comes to dispensing channels 222 can be varied by altering the length and shape of trigger channel 256 as appropriate. Thus, it is possible to delay the timing of the entry of liquid to dispensing channels 222 by appropriately adjusting the trigger channel 256. Or, in other words, the trigger channel 256 can also acts as a delaying channel.

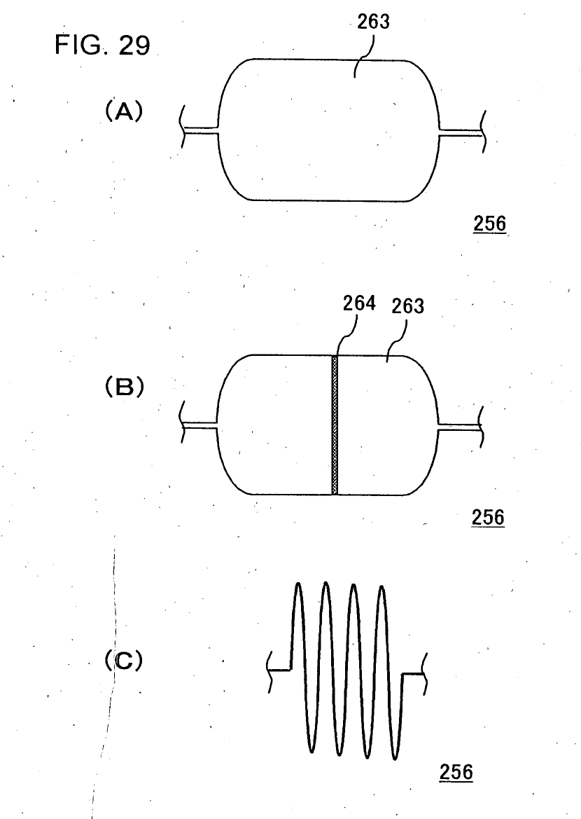

FIGS. 29A to 29C illustrate the exemplary structures of the trigger channel 256. The trigger channel 256 shown in FIG. 29A has an expanded channel region 263 along the path of trigger channel 256. The expanded channel region 263 serves as a delaying reservoir of the trigger channel 256 or acts as a delaying channel. Introduction of the expanded channel region can delay the timing at which the liquid switch portion 257 is opened.

The trigger channel 256 shown in FIG. 29B includes the same expanded channel region 263 with the one shown in FIG. 29A except that the region has a hydrophobic region 264. The hydrophobic region 264 is formed across the expanded channel region 263 in a direction perpendicular to the direction of flow in trigger channel 256, and this inhibits the flow of liquid along the inner wall of expanded channel region 263.

FIG. 29C shows a trigger channel 256 in the form of a zigzag line. With such a trigger channel, it is possible to alter the delay time, or to open the liquid switch portion 257 at a desired timing by adjusting the shape and length of the trigger channel 256. The shape of trigger channel 256 is not limited to the one shown in FIG. 29C, but may take, for example, a spiral form as long as it is sufficiently small in size.

According to the embodiment configured as described above, components are separated from a sample at the separation portion 218, the density of the components is homogenized at mixing portion 248, and the resulting liquid is distributed to dispensing channels 222. Thus, the liquid passed through the separation unit 213 is homogenized and then provided to each detection unit 214. Thus, it is possible to perform more precise, sensitive detection operation on the reaction for the test items selected by the opening and closing of the flow control units 314.

The trigger channel 256 having a delaying channel may be further modified such that its delaying time can be customized in accordance with a given request. Reaction which is necessary for detection requires a certain time, and mixture of one reagent component with another also requires another definite time. The delaying channel is introduced for ensuring such wait time. The wait time is different for each reaction and each operation. For a chip having a basic structure comprising plural analysis units (detection portions 214 or measurement units 233) to achieve plural analyses procedure different from each other, it is desirable to include a delaying channel that allows the setting of any desired delay time in accordance with the test. If the delaying channel described below is incorporated in the embodiment or other embodiments in the specification, it will be possible for those embodiments to set the wait time of delaying channel in accordance with the requirement from a given test.

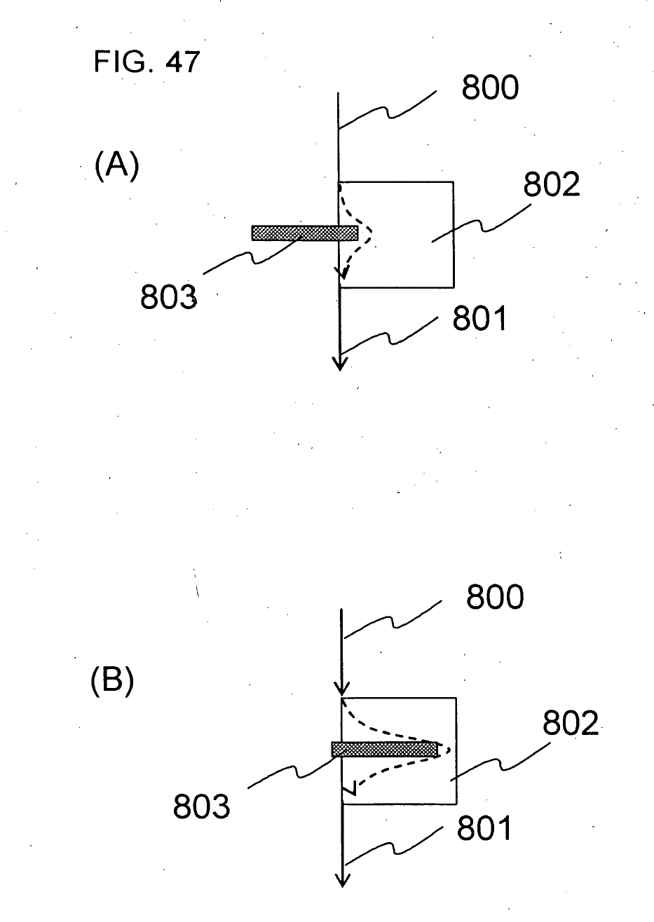

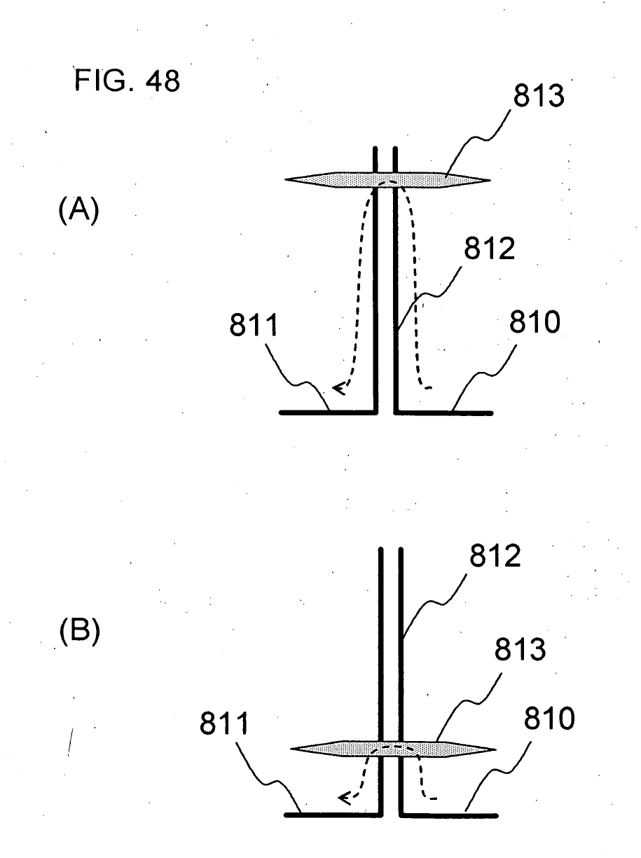

FIGS. 47A and 47B, and FIGS. 48A and 48B are top views for showing the structure of delaying channel capable of setting a desired delay time. The delaying channels shown in FIGS. 47A and 47B represent the modifications of the expanded channel regions 263 shown in FIGS. 29A and 29B of trigger channel 256.

The delaying channel shown in FIGS. 47A and 47B include, as its basic structure, an inflow channel 800, an outflow channel 801, and an expanded channel region 802. To customize the delaying channel shown in FIGS. 47A and 47B such that it gives a desired delay time, it is necessary to adjust the position of an interrupter for customizing 803. Formation of the interrupter for customizing 803 may be achieved by pressing a heater as depicted in FIG. 41 to an appropriate position of expanded channel region to deform a thermoplastic material there into a bulge that will act as an obstacle to the flow through the region. It is possible to obtain a desired delay time by changing the position of heater unit, thereby shifting the position of the interrupter for customizing 803 as appropriate.

Formation of the interrupter for customizing 803 may be achieved by pressing a stamp having a hydrophobic PDMS rubber pad or by applying hydrophobic ink onto an appropriate position of expanded channel region, thereby forming a hydrophobic coat there.

Protruding region of the interrupter for customizing 803 shown in FIG. 47A into the expanded channel region 802 is rather short, and thus current from inflow channel 800 can take a short course to reach outflow channel 801, which allows the current to pass the expanded channel region 802 in a short period. In contrast, when interrupter 803 protrudes more heavily towards the expanded channel region 802 as shown in FIG. 47B, current from inflow channel 800 must take a longer course to reach outflow channel 801, which causes the current to take a longer time to pass the expanded channel region 802. Thus, it is possible to customize in advance the delay time in accordance with a given test by adjusting the position of the interrupter for customizing 803. The number of the interrupter for customizing 803 is not limited to any specific one. For example, it is possible to lengthen the maximum delay time by providing two or more the interrupter for customizing 803 arranged in parallel within the expanded channel region 802.

FIGS. 48A and 48B show a different kind of delaying channel which alters the delay time by changing the length of channel. The delaying channel shown in FIGS. 48A and 48B includes an inflow channel 810, an outflow channel 811, and two extended connectors 812 connecting the two. When it is required to obtain a delaying channel with a desired delay time, a customizing channel 813 is formed between the two extended connectors 812 at an appropriately chosen level. Formation of a customizing channel 813 may be achieved by cutting a groove connecting the two extended connectors 812 using a thin, microscopic abrasion machine like the one used for dicing. The cutting edge of microscopic abrasion machine is so sharp that the notch of extended connector 812 coincides well with the profile of the customizing channel 813.

The customizing channel 813 may be a bridge made of a highly hydrophilic substance such as carboxymethylcellulose gel or agarose gel, which is placed between the two extended connectors 812. Since aqueous solution can move such a hydrophilic bridge by wetting it, the connection will be established between the two extended flow passages. Formation of such a bridge may be achieved by stamping a hydrophilic gel between the two passages, or by printing a sol form between the two passages to be dried later.

The customizing channel 813 shown in FIG. 48A is formed at a position far apart from the extended connector 812. In this case, current must flow a long distance as shown by the dotted line in the figure, thus lengthening the delay time. In contrast, the customizing channel 813 shown in FIG. 48B is formed close to the extended connector 812. Thus, the path for current to take for passing from inflow channel 810 to outflow channel 811 becomes short which reduces the delay time. Thus, it is possible to customize the delay time in accordance with a given test by forming a customizing channel 813 at an appropriate position using an abrasion cutter. Incidentally, for the delaying passage shown in FIGS. 48A and 48B, the two extended connectors 812 run parallel to each other with blind ends at their terminals. However, they may be in communication with each other at their distal ends. In short, they may take any form, as long as the form does not interfere with the function of customizing channel 813.

The above explanation has been given on the premise that the trigger channel 256 also includes a lag channel as shown in FIGS. 47A and 47B, and in FIGS. 48A and 48B. The embodiments shown in the figures can be attached to any specified channel or trigger channel of any chip described in the embodiment and other embodiments in the specification, and thereby make it possible to customize the delay time in accordance with a given test.

The basic structure of liquid switch portion of a chip representing the present embodiment can also be incorporated in the embodiments described below.

### Fourth Embodiments

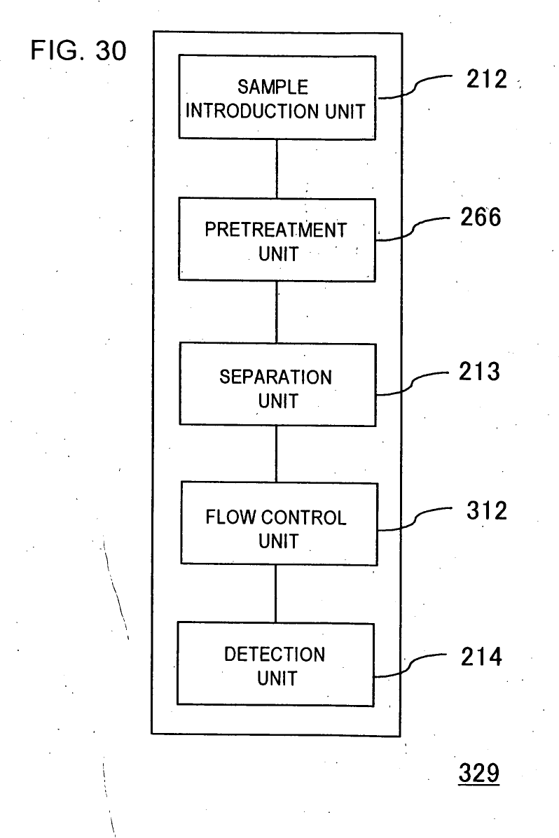

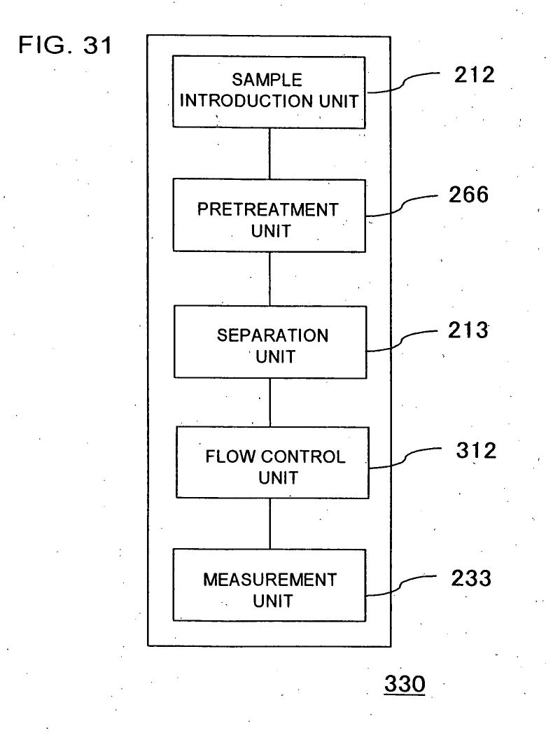

The chip described in relation to the first to third embodiments may further include a pretreatment unit for applying specified pretreatment on a sample before the sample is subjected to separation, at an intermediate stage between the sample introduction unit 212 and the separation unit 213 and the pretreatment unit can further include a flow control unit 314. FIGS. 30 and 31 are block diagrams for representing the functional components of chips representing this embodiment of the invention. The chips shown in FIGS. 30 and 31 include detection unit 214 and measurement unit 233 respectively as analysis unit. Both the chips 329 and 330 shown in FIGS. 30 and 31 include a pretreatment unit 266 between sample introduction unit 212 and separation unit 213. Description will be given below taking as an example a chip including a detection unit 214 like the one shown in FIG. 30.

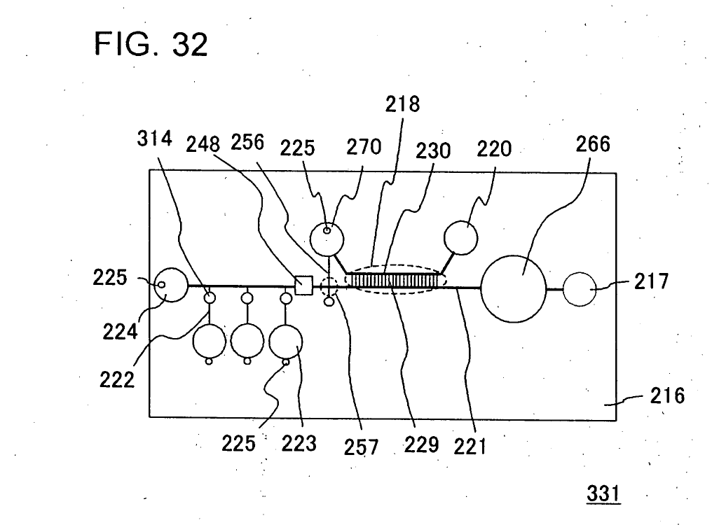

FIG. 32 is a diagram for representing the components of an exemplary chip that can be used as a chip 329. The chip 331 shown in FIG. 32 includes a pretreatment unit 266 with a control portion between inlet 217 and separation region 218. Pretreatment performed at pretreatment unit 266 may include the dissolution of extracellular components (for example, collagen), or reduction of the viscosity of a thick sample (for example, saliva or nasal secretion) to improve the fluidity of the sample.

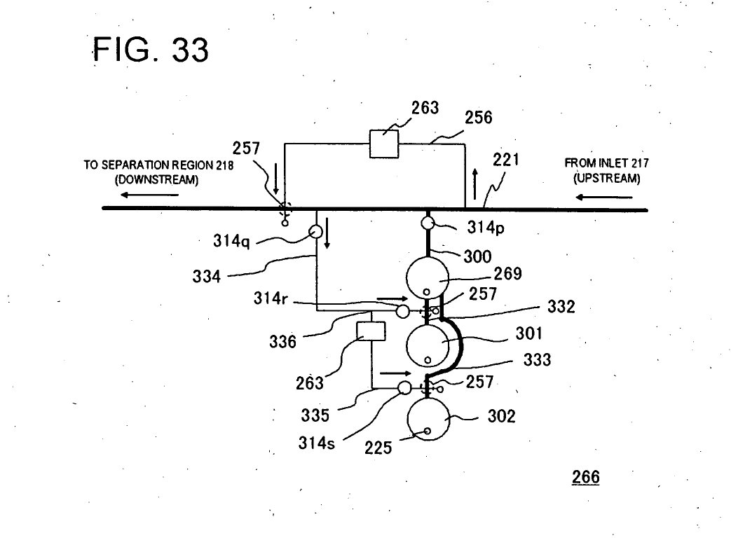

FIG. 33 is an enlarged view of the pretreatment unit 266 shown in FIG. 32. The pretreatment unit 266 includes a channel 300 in communication with main channel 221; a pretreatment reservoir 269 provided along the channel 300; channels 332 and 333 in communication with pretreatment reservoir 269; reagent reservoirs 301 and 302 in communication with channels 301 and 302 respectively; a trigger channel 334 branched from the main channel 221 at the downstream side of the channel 330 and in communicating with the channel 332; a trigger channel 256 which is branched from the trigger channel 334 at the branching portion 336, providing an expanded channel region 263 as the time delaying reservoir thereto, interflowing to the main channel 221 via the liquid channel portion 257 at the downstream side of trigger channel 334, and provided the expanded channel region 263 thereto.

The pretreatment unit 266 further includes flow control units 314p, 314q, 314r, and 314s on the passage 300, trigger channel 334 upstream of the branching potion 336, trigger channel 334 at downstream of the branching portion 336, and trigger channel 335, respectively. Each of the pretreatment reservoir 269, the reagent reservoir 301, the reagent reservoir 302, trigger channel 256, trigger channel 334 and trigger channel 335 is provided with a vent hole 225.

Since the pretreatment unit 266 includes flow control units 314p and 314q, it can perform pretreatment operation at a pretreatment reservoir 269 at a single step or two separate steps. Or if it is required to do so, the pretreatment unit 266 can omit the execution of pretreatment.

(a) Pretreatment at Pretreatment Reservoir 269 is not Executed

The flow control units 314p and 314q of pretreatment unit 266 should be closed. The structure of flow control units 314p and 314q may be the same as that of the corresponding flow control portion of the first embodiment. When flow control units 314p and 314q are closed, sample flowing through main channel 221 cannot advance to the pretreatment reservoir 269 or to channel 332. The sample will pass by the pretreatment unit 266 leaving it alone.

Sample is blocked at the liquid switch portion 257 provided on main channel 221. Alternatively, some part of sample enters trigger channel 256 from main channel 221 and moves along the circuit to reach the liquid switch portion 257. At that moment, the liquid switch portion 257 is opened in the manner as described in relation to the third embodiment. Thus, the sample restarts to flow along main channel 221 towards the separation region 218. In this case, the expanded channel region 263 may be set to give a minimum delay, or the trigger channel 256, the liquid switch portion 257, the expanded channel region 263, and the liquid switch portion 257 may be omitted in acvance.

(b) One Step Pretreatment is Executed at Pretreatment Reservoir 269

In this case, flow control unit 314p on channel 300, and flow control units 314q and 314r are opened, and flow control unit 314s is closed.

Since flow control unit 314p on channel 300 is open, a sample introduced via inlet 217 into the system runs past main channel 221 and channel 300 to enter pretreatment reservoir 269. The pretreatment reservoir 269 is a well for applying specified pretreatment to sample introduced via inlet 217. The pretreatment reservoir 269 may include in advance a reagent (not shown) necessary for pretreatment such as an enzyme, for example, collagenase, lysozyme chloride and so on. If the pretreatment consists of certain operation such as incubation, introduction of a pretreatment reagent is not necessary.

The reagent reservoir 301 may include the same volume of buffer with the capacity of pretreatment reservoir 269. Sample, after having undergone pretreatment at pretreatment reservoir 269, must flow back to main channel 221. For this purpose, the level of liquid in the reagent reservoir 301 should be maintained as high as or higher than the liquid in the main channel 221.

The liquid switch portion 257 provided between pretreatment reservoir 269 and reagent reservoir 301 allows reagent reservoir 301 to hold buffer by taking the structure shown in FIG. 53 which will be described later.

When a pretreatment reagent is introduced into pretreatment reservoir 269, it reacts with a reagent put in advance to produce a specified pretreatment reaction. Incidentally, a part of the sample move from the pretreatment reservoir 269 to the channels 332 and 333, and is dammed at the liquid switch portions 257 provided to the channels 332 and 333, respectively.

Part of reagent moves along channel 300, past main line 221, enters trigger channel 334 on the channel connecting the pretreatment reservoir 269 and the reagent reservoir 301, and reaches liquid switch portion 257 to open it. Then, buffer in the reagent reservoir 301 flows upstream towards pretreatment reservoir 269 and pushes the content of pretreatment reservoir 269 towards main channel 221. The delay time the trigger channel 334 requires for opening the liquid switch portion 257 is adjusted to be longer than the time required for pretreatment reaction. For this purpose, an expanded channel region may additionally be introduced into the loop of the trigger channel 334.

One other part of reagent enters from main channel 221 into the trigger channel 256 and reaches liquid switch portion 257. When the sample in trigger channel 256 reaches the liquid switch portion 257, the liquid switch portion 257 opens, and thus sample in main channel 221 starts to flow again towards separation region 218. The trigger channel 256, expanded channel region 263, and liquid switch portion 257 are provided to close main channel 221 until sample has been pretreated sufficiently in pretreatment reservoir 269. The delay time introduced by expanded channel region 263 should be adjusted to be sufficiently long as to allow the pretreatment reservoir 269 to be filled with sample.

(c) Two Step Pretreatment is Executed at Pretreatment Reservoir 269

Two step pretreatment may include, for example, a first step of decomposing extracellular substances such as collagen to allow thereby cells (for example, insulin cells, glucagon cells) in a sample (for example, tissue such as Langerhans' islands) to deposit at the base of reaction reservoir, and a second step of applying a chemical agent (for example, glucose) to the precipitated cells, recovering the product (for example, insulin) secreted by the cells as a result of reaction, and transporting the product to main channel.

In this case, flow control unit 314p on channel 300, and flow control units 314q, 314r and 314s are opened. The pretreatment reservoir 269 may include in advance a reagent (for example, freeze-dried collagenase) as needed. The reagent reservoir 301 may also include a reagent or a buffer (for example, glucose solution) necessary for the second step reaction. The reagent reservoir 302 may include buffer that pushes back the sample having undergone reaction towards the main channel. The level of liquid in the first and reagent reservoirs 301 and 302 should be maintained as high as or higher than the liquid in the main channel 221. The reagent reservoirs 301 and 302 may include a volume of buffer as large as or larger than the capacity of pretreatment reservoir 269.

Sample introduced via inlet 217 enters the pretreatment reservoir 269 to fill it. Then the first step reaction (for example, exposure of cells as a result of the dissolution of collagen and deposition of cells) occurs. The sample advances further along main channel 221 and a part of the sample turns aside to enter trigger channel 334. After a sufficiently long delay for the completion of first step reaction which is generated by the sample running along the trigger channel 334, the liquid switch portion 257 between pretreatment reservoir 269 and reagent reservoir 301 is opened. Then, a reagent (for example, glucose solution) necessary for the second step reaction and stored in reagent reservoir 301 moves to pretreatment reservoir 269, and pushes out the liquid resting in pretreatment reservoir 269 to displace the liquid there completely. The liquid pushed into main channel flows upstream along main channel because the liquid switch portion 257 provided to a downstream point of main channel is still closed.

After a sufficiently long delay for the completion of second step reaction (for example, reaction in which insulin cells produce insulin as a response to glucose solution), the liquid switch portion 257 between pretreatment reservoir 269 and reagent reservoir 302 is opened. Then, buffer stored in the reagent reservoir 302 pushes out the content (for example, glucose solution containing insulin produced by cells) of pretreatment reservoir 269, and transports the content as a new sample to main channel 221.

Another part of sample enters from main channel 221 into trigger channel 256 and runs along the latter to reach liquid switch portion 257. When the sample in the trigger channel 256 reaches the liquid switch portion 257, the liquid switch portion 257 opens, and the sample having undergone pretreatment advances towards separation region 218.

As seen from above, it is possible according to the chip of the invention to allow the first and second step reactions to occur dependent on the configuration of the chip itself without requiring any external control unit for the purpose.

### Fifth Embodiment

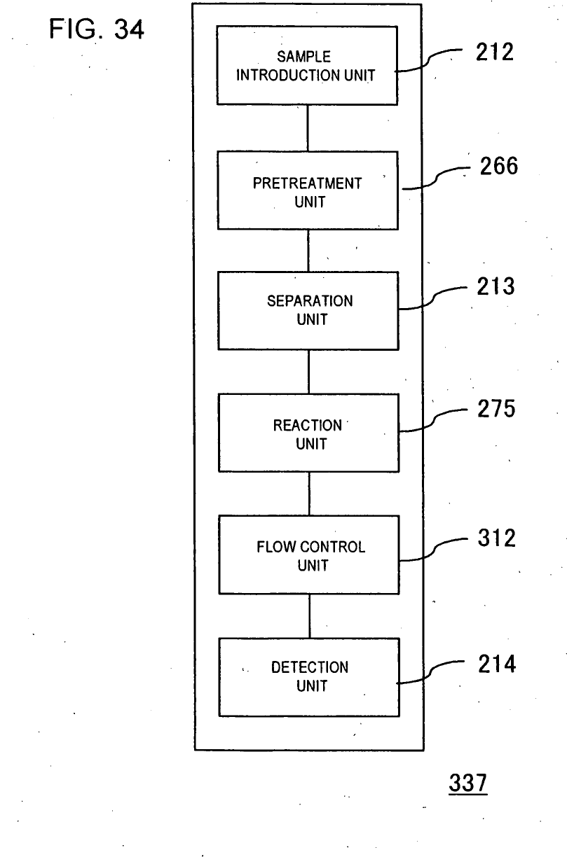

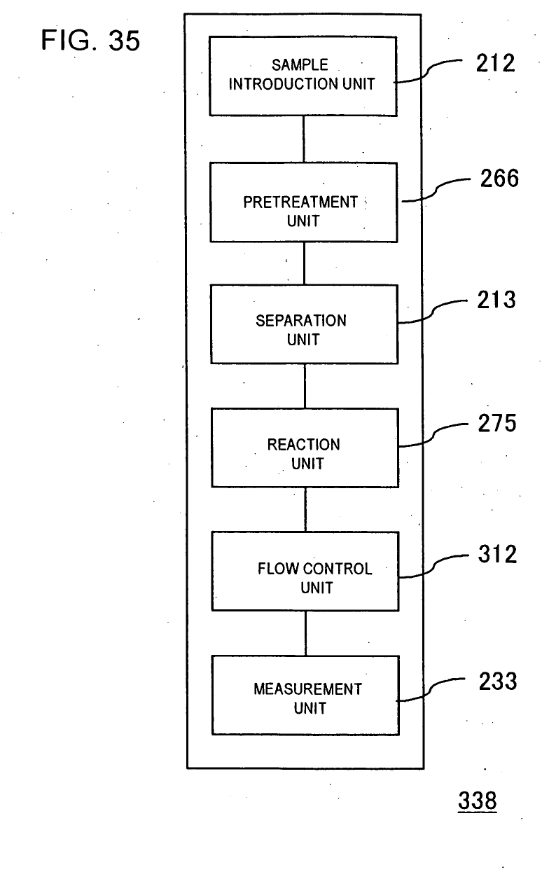

The chip described in relation to the above embodiments may further include a reaction unit 275 between the separation unit 213 and the flow control unit 312, and the reaction unit may further include a flow control unit 314. FIGS. 34 and 35 are block diagrams for representing the functional components of chips representing this embodiment of the invention. The chips shown in FIGS. 34 and 35 include detection unit 214 and measurement unit 233 respectively as analysis unit. Both the chips 337 and 338 shown in FIGS. 34 and 35 include a reaction unit 275 between separation unit 213 and flow control unit 312.

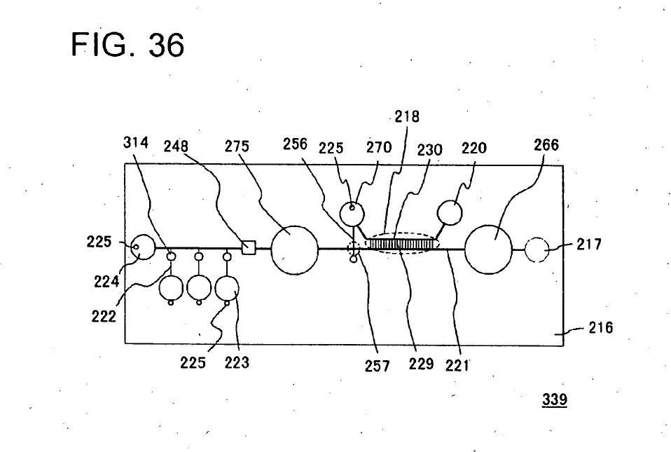

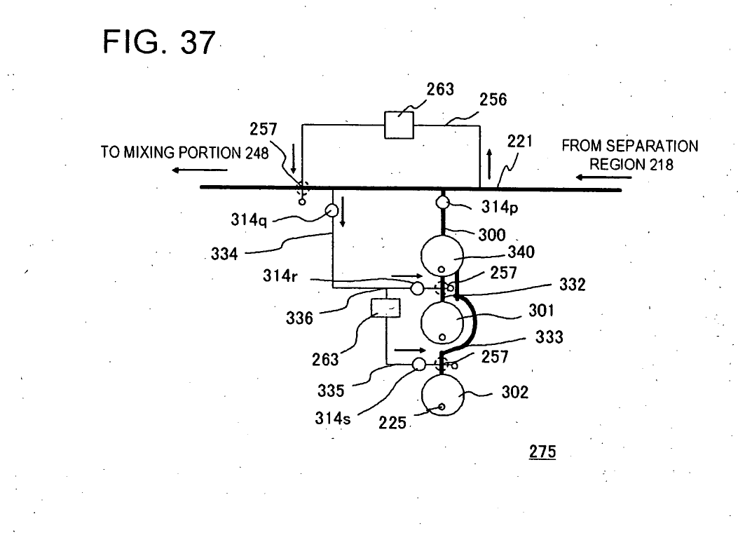

Description will be given below taking as an example a cofiguration corresponding to the chip 337 shown in FIG. 34. FIG. 36 is a diagram for representing the components of an exemplary chip that corresponds to a chip 337. The chip 339 shown in FIG. 36 includes a reaction unit 275 between separation portion 218 on main channel 221 and dispensing channels 222. FIG. 37 illustrates the configuration of reaction unit 275 shown in FIG. 36. The reaction unit 275 shown in FIG. 37 is similar in its basic structure to the pretreatment unit 266 shown in FIG. 33 except that it includes a reaction reservoir 340 instead of a pretreatment reservoir 269.

The reaction reservoir 340 shown in FIG. 37 is a well for allowing an element contained in sample and separated at the separation region 218 to undergo a specified reaction. Like the pretreatment unit 266 described in relation to the fourth embodiment, the reaction unit 275 includes flow control units 314p, 314q, 314r, and 314s. It is possible to achieve, at the reaction unit 275 like the foregoing pretreatment portion, no reaction, one-step reaction or two-step reaction by setting an opening and closing the flow control portions appropriately. Exemplary one step reactions may include solubilization of cells, mixing of reagents and so on, while exemplary two step reactions may include recovery of a product secreted by cells such as insulin or the like. The treatment step is similar to that observed with the pretreatment unit 266.

For one step pretreatment to be executed at reaction portion, flow control unit 314p on reaction unit 275, and flow control units 314q and 314r are opened, and flow control unit 314s is closed. The reaction reservoir 340 may include in advance a surfactant for solubilizing the membrane of cells which is constituted of lipids membrane, and freeze-frozen lipase for decomposing lipids, while a reagent reservoir 301 may contain buffer.

The reaction unit 275 may further includes another separation region 218. Then, sample having undergone reaction is transported to the separation region 218 downstream of the reaction reservoir 340 where further separation is executed. Then, if sample having undergone solubilization reaction still contains some insoluble molecules, those insoluble molecules will be eliminated during the passage of the sample through the separation region 218 provided downstream of reaction reservoir 340.

The above explanation has been given on the premise that the reaction unit 275 includes the reagent wells 301, 302 connected in series. However, the reaction unit 275 may include three or more reagent reservoirs. The above exemplary chip includes a single reaction unit 275. However, the chip may include two or more reaction portions 275.

### Sixth Embodiment

The chip described above in relation to the foregoing embodiments may include an analysis unit (detection unit 214 or measurement unit 233) connected to a reservoir where a flow control unit 312 is provided to the connecting channel. With a chip having a configuration as described above, it is possible to select any desired one among one step reaction to multi-step reaction as appropriate in accordance with the requirement from a given test. The configuration of analysis portion can be varied so as to allow any typical reactions to occur to meet any general-purpose applications as will be described later in relation of an eighth embodiment. If a number of such general-purpose analysis portions different in their property are combined on a chip, the chip will command profitably wide applicability.

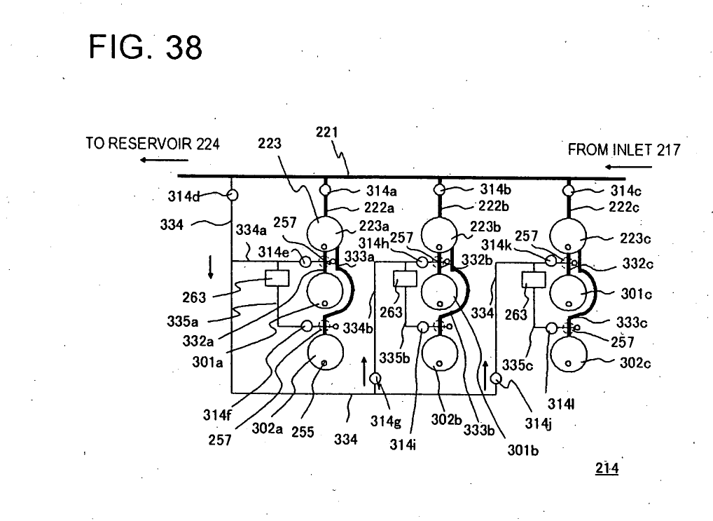

Description will be given below taking as an example a detection unit 214 that allows predetermined-step(s) reaction to occur. Description will be given below with reference to figures about a detection unit 214 having a single detection reservoir 223. However, as described later with reference to FIG. 38, the detection unit 214 may include two or more detection reservoirs 223 and peripheral members.

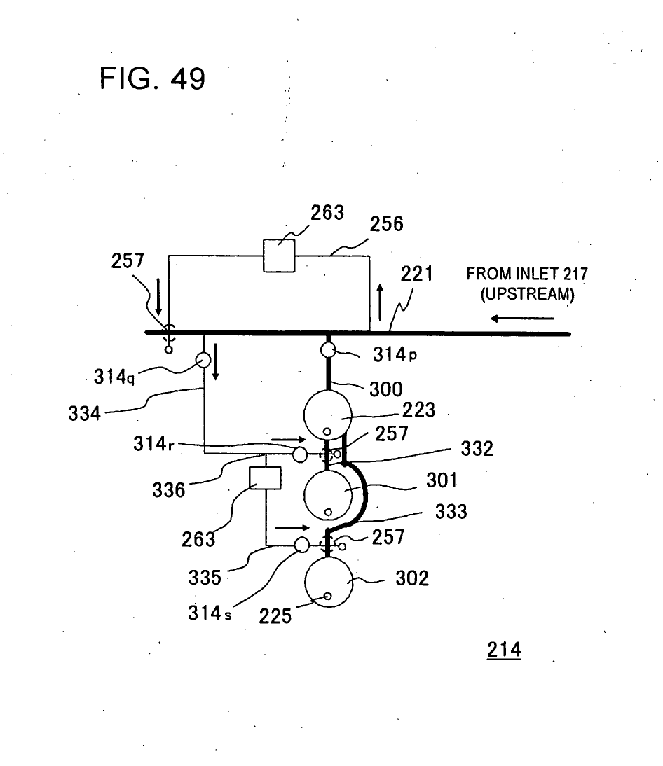

FIG. 49 illustrates the configuration of a detection unit 214. The detection unit 214 shown in FIG. 49 is similar in its basic structure to the pretreatment unit 266 shown in FIG. 33 except that it includes a detection reservoir 223 instead of a pretreatment reservoir 269.

The detection reservoir 223 shown in FIG. 49 is a well for allowing an component of a sample introduced via inlet 217 to undergo a specified detection reaction. Like the pretreatment unit 266 described in relation to the fourth embodiment, the detection unit 214 includes a flow control unit 314. It is possible to achieve, at the detection unit 214, one step reaction to two step reaction by setting an opening and closing of the flow control unit 314 appropriately, or to omit such reaction altogether.

Sample having undergone separation operation at separation region 218 enters detection reservoir 223 as needed, and undergoes specified reaction. Downstream of the detection reservoir 223, there is provided a liquid switch portion 257, and thus downstream movement of liquid in detection reservoir 223 beyond the liquid switch portion 257 is prohibited. It is possible to alter the layout of trigger channel 256 so as to give a delay sufficiently long to allow the detection reaction occurring at detection reservoir 223 to be completed. If the detection reaction takes a long time, the expanded channel region 263 may be enlarged. It is also possible for the trigger channel 256 to include a lag channel thereby altering the delay time to any desired length of time.

The above explanation has been given with respect to FIG. 49 on the premise that the detection reservoir 223 includes reagent reservoirs 301, 302 connected in series. However, the detection reservoir 223 may include a single reagent reservoir 301, or three or more reagent reservoirs.

FIG. 58 shows a variation of the detection unit shown in FIG. 49 in which the detection reservoir includes a single reagent reservoir 301. For detection reaction to occur at the detection reservoir 223 shown in FIG. 58, it is necessary to open flow control units 314p and 314q. In contrast, to prohibit detection reaction at detection reservoir 223, it is necessary to close flow control units 314p and 314q.

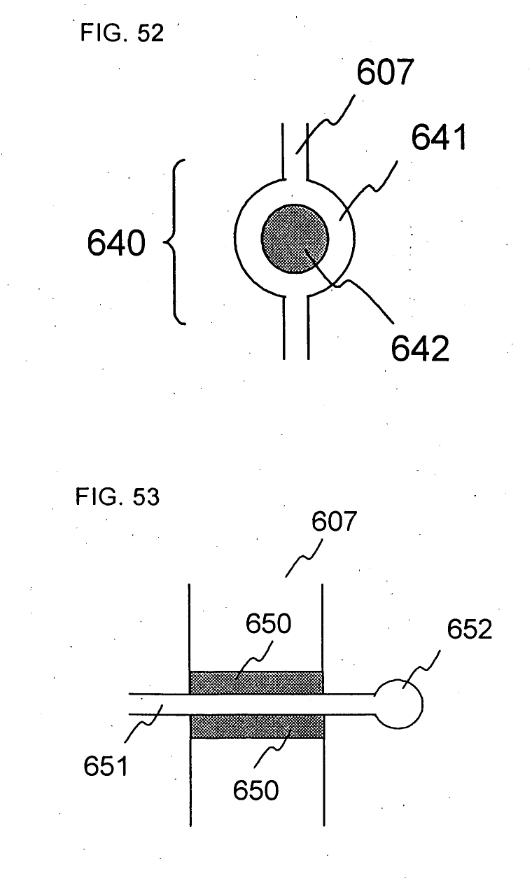

The detection portion shown in FIG. 58 includes a closing switch 640 between flow control unit 314p on the passage 300 and the detection reservoir 223. FIG. 52 is a top view for showing the structure of the closing switch 640 provided to the detection portion. The closing switch 640 is provided for preventing the upstream flow of sample having undergone reaction from the reservoirs towards main channel 221. The closing switch 640 includes an expanded portion 641 provided to the channel and a bulging body 642 embedded in the expanded portion. When liquid passes through the channel 607 and the expanded portion 641, the bulging body 642 bulges slowly as a result of the interaction with the liquid until it completely occludes the expanded portion 641 that enables the delayed occlusion of the channel 607.

Suitable materials for the bulging body 642 may include dried and contracted polyacrylamide, beads consisting of a water-absorbing polymer. The bulging body 642 is stabilized in the expanded portion 641 due to its diameter being larger than that of the channel 607, adhesion to part of the expanded portion 641 and so on.

Turn back again to FIG. 58. It is possible to simplify the structure of the system by allowing a detection reservoir 223 to have a single reagent well 301. For example, a chip where one-step detection reaction is allowed to occur may have such a simplified structure. About the detection portion shown in FIG. 58, description has been given on the premise that the detection unit includes two of the flow control units 314p, 314q, one is on the channel 300 and the other is on the trigger channel 334. However, it is only necessary to provide at least the flow control unit 314p. It is possible to more reliably inhibit the wasteful consumption of sample by providing flow control unit 314q to trigger channel 334.

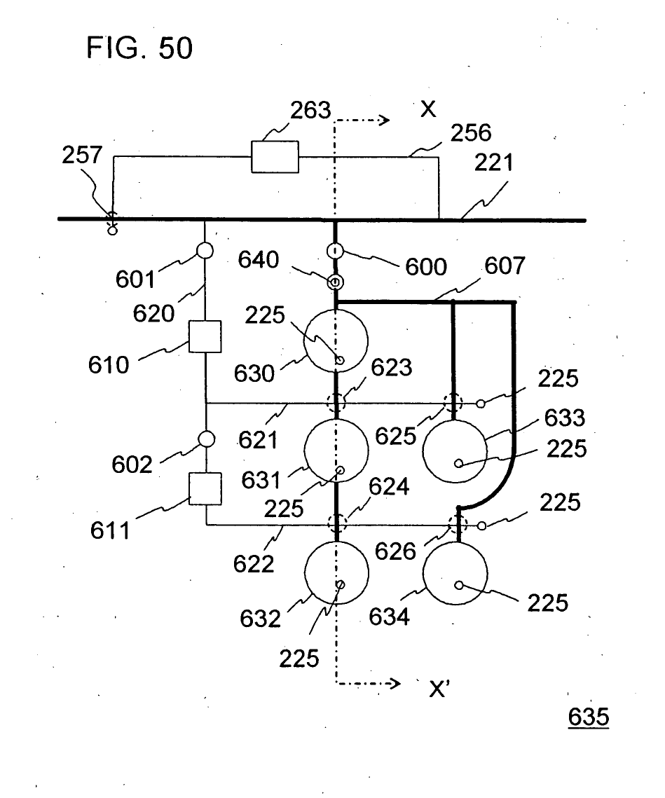

For example, the detection unit 214 may include five reservoirs. These reservoirs are used as detection reservoir, waste reservoir, reagent reservoir, buffer reservoir and so on, according to the kind of detection reactions. FIG. 50 is a plane view for showing the configuration of another detection unit 635. The detection unit 635 shown in FIG. 50 includes a reservoir group consisting of five reservoirs 630, 631, 632, 633 and 634; a channel 607 connecting the reservoir group to main channel 221; a liquid switch portion group consisting of four liquid switch portions 623, 624, 625 and 626; an assembly of trigger channels consisting of the trigger channels 620, 621 and 622 connecting the liquid switch portion group to the main channel 221; lag channels 610 and 611 provided to the trigger channels; and flow control portions 600, 601 and 602 responsible for the opening and closing of channel 607 and trigger channels.

The detection unit may further include, although it is not absolutely necessary, a trigger channel 256, an expanded channel region 263, and a liquid switch portion 257, in order to close main channel 221 until sample fills the reservoir 630.

Each of the five reservoirs 630, 631, 632, 633 and 634 and the reservoir 635 has a vent hole 225. The reservoir 630 is mainly used as a detection reservoir. The reservoirs 631 and 632 are mainly used for storing waste. The reservoirs 633 and 634 are mainly used as reagent reservoirs for supplying reagent to reservoir 630.

To main channel 221, there are provided from upstream downward in order a bifurcation for trigger channel 256, bifurcation for channel 607, bifurcation for trigger up-loop circuit 620, and bifurcation (that is to say, liquid switch 257) for the downstream limb of trigger channel 256.

To the stem channel 607 bifurcating from main channel 221 there are provided from upstream downward in order a flow control portion 600, closing switch 640, bifurcation for first limb channel 607, reservoir 630, liquid switch portion 623, reservoir 631, liquid switch portion 624, and reservoir 632. To one second limb channel 607 bifurcated from first limb detection passage, there are provided from upstream in order a liquid switch portion 625 and reservoir 633. To other second limb detection passage bifurcated from first limb detection passage, there are provided from upstream in order a liquid switch portion 626 and reservoir 634.

The trigger channel 620 includes from upstream downward in order a flow control unit 601 and a lag channel 610, and bifurcates at a downstream end to produce a trigger channel 621 and another trigger channel 622. To the trigger channel 621 there are provided from upstream downward in order a liquid switch portion 623 and another liquid switch portion 625. The distal end of the trigger channel 621 communicate with a vent hole 225. To the trigger channel 622 there are provided from upstream downward in order a flow control portion 602, lag channel 611, liquid switch portion 624, and liquid switch portion 626. The distal end of trigger limb 622 communicates with another vent hole 225.

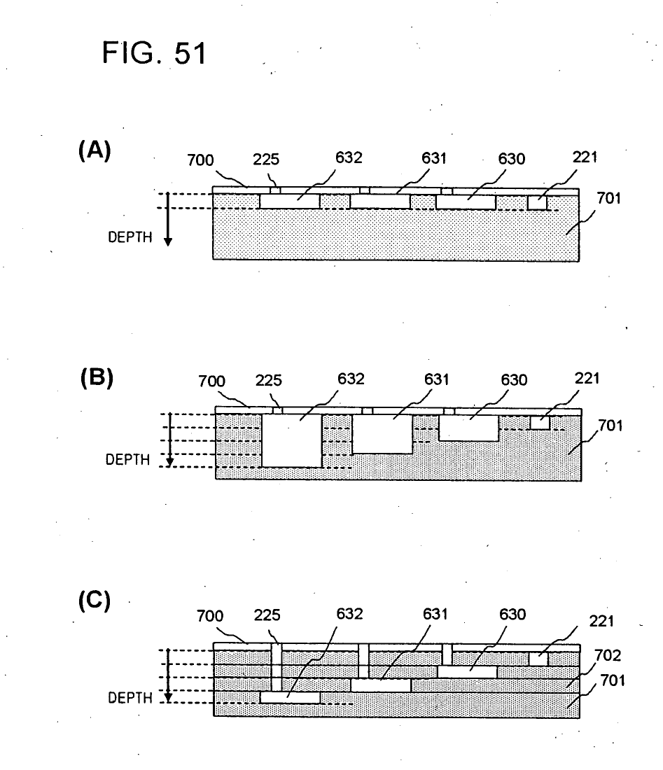

FIGS. 51A to 51C are sectional views for showing the structure of a chip having a detection unit 635 as depicted in FIG. 50. FIGS. 51A to 51C show the cross-sections of the detection portion cut along line X-X′ of FIG. 50. The chip whose cross-sections are shown in FIGS. 51A to 51C includes a substrate 701 and a lid 700. All the channel systems formed on the substrate 701 have the same depth with that of main channel 221 excepting vent holes 225. The lid 700 has vent holes 225 so as to communicate with the reaction reservoirs. Reagents necessary for detection reactions are distributed to the reservoirs 630 to 634 formed on the substrate 701, and flow control portions 600, 601 and 602, and delay time of a lag channel 610, 611, 612 are adjusted in accordance with a given test (customized). Then, the lid 700 is joined to the substrate 701.

With the chip shown in FIG. 51A, reservoirs 630 to 634 have the same depth with that of main channel 221. Since liquid is driven via capillary action promoted by the hydrophilic property of the channels, driving force is given even if they have the same depth.

If difference in water level is utilized in addition to capillary action, it will be possible to move liquid more speedily. FIG. 51B shows the cross-section of a chip in which water-level difference is also utilized. To utilize water-level difference, four kinds of channels are prepared that have four different depths ranked as the shallowest level zero to the deepest level 3. In the particular example shown in the figure, the main channel 221, the trigger channel group and the lag channel group have a depth of level zero; the reservoir 630 has a depth of level 1; and the reservoirs 631 and 632 which mainly serve as waste reservoirs have a depth of level 2 and 3, respectively. Because of the difference in depth, water-level difference is produced between different kinds of reservoirs after the reservoirs are filled with liquid as a result of capillary action. Particularly force driving liquid from the main channel 221 to the reservoir 632 is produced. Although not shown in the figure, the reagent reservoirs 633 and 634 used for supplying the reagents are formed to have a depth of level zero.

With the chip shown in FIG. 51B, the reservoir has a different volume according to its depth. It is possible to equalize the volumes of all reservoirs, thereby ensuring the economic use of reagents necessary for measurement. FIG. 51C is a modification of the chip shown in FIG. 51B. With the chip of FIG. 51C, reservoirs 630, 631 and 632 have the same volume. The substrate of the chip shown in FIG. 51C has a stacked structure comprising four thin substrates 702 and one common substrate 701. Through-holes are formed at appropriate positions on each thin substrate 702, and then they are joined to allow the through-holes to be put together to form thereby some of the reservoir group, assembly of channel 607, trigger channel group, the vent holes 225. Although not shown in FIG. 51C, the trigger channel group has a depth of level zero as does the vent hole 225, but after forming a liquid switch portion it extends downward to communicate with a reservoir. To the top surface of chip, a lid 700 having vent holes 225 formed is joined as shown in FIG. 51B.

As noted above, suitable materials of a chip include transparent materials, for example, resins such as PET or PMMA, quartz, glass, and so on. To utilize capillary effect for the transportation of liquid, the interior of liquid transporting channel system is preferably made of a hydrophilic material. If the interior of the channel is made of a hydrophobic material such as PMMA, it is preferable to treat the channel with a surface treatment agent such as MPC or acrylamide gel to apply coating there, thereby to hydrophilize for providing with the higher hydrophilicity. For the liquid switch portion and the like that has a hydrophobic surface, hydrophobic treatment may be applied to the channel surface having be hydrophilized.

FIG. 53 gives a top view for showing the basic structure of the liquid switch portions 623 to 626 of detection unit 635 shown in FIG. 50. As shown in FIG. 53, each of liquid switch portions 623 to 626 includes a channel 607, a trigger channel 651, a damming portion 650, and vent hole 652 provided to the distal end of the trigger channel 651. The vent hole 652 corresponds to vent hole 225 of FIG. 50. The trigger channel 651 corresponds to trigger channel 621 or 622 of FIG. 50.

The liquid switch portion shown in FIG. 53 is different from the corresponding switch portion described above in relation to the foregoing embodiments in that damming portions 650 are arranged on both sides of trigger channel 651. Since two damming portions 650 are arranged on both sides of trigger channel 651, it is possible to prevent from liquid in trigger channel 651 from entering into channel 607, even when no liquid is present in channel 607. If one limb of channel 607 is filled with liquid and the other limb is empty, the liquid in the former limb will not invade the latter because of damming portions 650. However, as soon as the trigger channel 651 is filled with liquid, the two limbs of channel 607 will communicate with each other as in the above-described liquid switch portions.

It is possible to allow any of single to multi-step detection reactions to occur at detection unit 214 by using the chip shown in FIGS. 50 to 53. Even if the chip includes a measurement unit 233 instead of a detection unit 214, it is possible to allow sample to undergo any of single to multi-step reactions before measurement by constructing the separation portions in the manner as described above. Practice of clinical biochemical test using the chip as shown in FIGS. 50 to 53 will be described in relation to an eighth embodiment.

In the above description, attention has been paid to detection reaction occurring at a single detection reservoir 223 to explain the function of detecting portion 214. Next, an embodiment where the detection unit 214 includes three detection reservoirs 223 will be described. However, the number of detection reservoirs 223 is not limited to any specific number, and may be two or four or more.

FIG. 38 shows the configuration of a detection unit 214 of this embodiment. The detection unit 214 includes from downstream to upward in order three dispensing channels 222a, 222b, 222c which are in communication with detection reservoirs 223a, 223b, 223c, respectively. The dispensing channels 222a to 222c have respective flow control units 314a to 314c, respectively.

To a detection reservoir 223a are connected a reagent reservoir 301a via a channel 332a, and a second reagent reservoir 302a via a channel 333a. Similarly, to a detection reservoir 223b are connected a reagent reservoir 301b via a channel 332b, and a second reagent reservoir 302b via a channel 333b. To a detection reservoir 223c are connected a reagent reservoir 301c via a channel 332c, and a second reagent reservoir 302c via a channel 333c.

A trigger channel 334 branches off at a point of main channel 221 downstream of dispensing channel 222a. Trigger channel 334 has a flow control unit 314d. Trigger passage 334 branches off downstream of flow control unit 314 to produce trigger channel 334a connects via a liquid switch portion 257 to the channel 332a, and a trigger channel 335a connects via a liquid switch portion 257 to the channel 333a. The trigger channel 334 connects via the liquid switch portion 257 to the channel 332c. Trigger channel 335a branches off from trigger channel 334a. Trigger channel 334a has flow control unit 314e downstream of the bifurcation point of the trigger channel 335a. Sub-passage 335a has a expanded channel region 263 and a flow control unit 314f.

The trigger channel 334b branches off from trigger channel 334 at a point downstream of the foregoing branching off point from which sub-passage 334a branches off. The trigger channel 334b shoots out a trigger channel 335b. The trigger channel 334b has one flow control unit 314g upstream of the bifurcation point of the trigger channel 335b, and another flow control unit 314h downstream of the bifurcation point. The trigger 335b has an expanded channel region 263 and a flow control unit 314i. Trigger channel 334b connects via the liquid switch portion 257 to the channel 332b. The trigger channel 335b connects via another liquid switch portion 257 to the connector channel 333b.

From stem-passage 334, sub-passage 335c branches off at a point downstream of the point where sub-passage 334b branches off. Stem-passage 334 has flow control unit 314j between the two branching-off points: one for sub-passage 334b and the other for sub-passage 335c. Stem-passage has another flow control unit 314k at a point downstream of the point where sub-passage 335c branches off. Sub-passage 335c has expanded channel region 263 and flow control unit 314l. Stem-passage 334 connects via liquid switch portion 257 to connector channel 332c. Sub-passage 335c connects via another liquid switch portion 257 to connector channel 333c.

According to the configuration of detecting portion 214 shown in FIG. 38, it is possible to alter the number of active detection reservoirs as appropriate by opening/closing flow control units 314a to 314l. It is also possible to determine whether a given detection reservoir gives one step reaction or two step reaction.

Table 1 shows the layout of flow control portions required for the activation of detection reservoirs 223a to 223c. The table shows the open/closure condition of the individual flow control units 314a to 314l for each of the detection reservoirs 223a to 223c, which depends on the usage of the reagent reservoirs 301a to 301c and the reagent reservoirs 302a to 302c. In Table 1, as for the detection reservoirs 223a to 223c, the reagent reservoirs 301a to 301c and the reagent reservoirs 302a to 302c, a reservoir marked by open circle (‘∘’) is to be used while a reservoir marked by cross (‘x’) is not to be used. Also, as for the flow control portions 314a to 314l, a flow control portion marked by open circle (‘∘’) is to be open while a flow control portion marked by cross (‘x’) is to be closed. In the table, the blank portion for the flow control units indicates that the opening and closing state thereof is variable in accordance with the state of use for other detection reservoirs and the reservoirs.

Table 1

TABLE 1

| | | | 314a | 314b | 314c | 314d | 314e | 314f | 314g | 314h | 314i | 314j | 314k | 314l |
|---|---|---|---|---|---|---|---|---|---|---|---|---|---|---|
| 223a◯ | 301a◯ | 302a◯ | ◯ | | | ◯ | ◯ | ◯ | | | | | | |
| 223a◯ | 301a◯ | 302aX | ◯ | | | ◯ | ◯ | X | | | | | | |
| 223a◯ | 301aX | 302a◯ | ◯ | | | ◯ | X | ◯ | | | | | | |
| 223a◯ | 301aX | 302aX | ◯ | | | ◯ | X | X | | | | | | |
| 223aX | 301aX | 302aX | X | | | ◯ | X | X | | | | | | |
| 223b◯ | 301b◯ | 302b◯ | | ◯ | | ◯ | | | ◯ | ◯ | ◯ | | | |
| 223b◯ | 301b◯ | 302bX | | ◯ | | ◯ | | | ◯ | ◯ | X | | | |
| 223b◯ | 301bX | 302b◯ | | ◯ | | ◯ | | | ◯ | X | ◯ | | | |
| 223b◯ | 301bX | 302bX | | ◯ | | ◯ | | | ◯ | X | X | | | |
| 223bX | 301bX | 302bX | | X | | ◯ | | | X | X | X | | | |
| 223c◯ | 301c◯ | 302c◯ | | | ◯ | ◯ | | | | | | ◯ | ◯ | ◯ |
| 223c◯ | 301c◯ | 302cX | | | ◯ | ◯ | | | | | | ◯ | ◯ | X |
| 223c◯ | 301cX | 302c◯ | | | ◯ | ◯ | | | | | | ◯ | X | ◯ |
| 223c◯ | 301cX | 302cX | | | ◯ | ◯ | | | | | | ◯ | X | X |
| 223cX | 301cX | 302cX | | | X | ◯ | | | | | | X | X | X |

DETECTION RESERVOIRS AND RESERVOIRS
OPEN CIRCLE: OPEN
CLOSS: CLOSED
FLOW CONTROL UNITS
OPEN CIRCLE: USED
CLOSS: NOT USED
NO MARK: DEPENDS ON THE USE CONDITION OF OTHER DETECTION RESERVOIRS

For example, it is possible to determine the level of insulin in plasma by utilizing multi-step reaction at a detection unit 214. Explanation will be given on the premise that only detection reservoir 223a is used for this purpose. To activate only detection reservoir 223a, flow control units 314a, 314d, 314e and 314f are opened. On the other hand, flow control units 314b, 314c and 314g-314l are closed.

Anti-insulin antibody which serves as a primary antibody is immobilized in advance on the surface of detection reservoir 223a. Liquid containing anti-insulin antibody to which an enzyme for coloring reaction (“enzyme-linked antibody” hereinafter) is attached is stored in reagent well 301a. Liquid containing a reagent which develops color under the influence of the enzyme is stored in reagent well 302a.

On the chip prepared as described above, a sample is allowed to flow along the main channel 221. The sample is guided to the detection reservoir 223a. Part of the sample enters into the trigger channel 334 at a point downstream of a branching point to detection reservoir 223a. The sample entering trigger channel 334 takes a certain time to reach via the trigger channel 334a to open the liquid switch portion 257. During this time, the sample is allowed to contact with the liquid in detection reservoir 223a where insulin in the sample interacts specifically with anti-insulin antibody immobilized on the surface of the detection reservoir 223a.

As noted above, a part of the sample enters trigger channel 334 and reaches the liquid switch portion 257 at the predetermined time. Then, the liquid switch portion 257 is opened, and enzyme-linked antibody stored in reagent reservoir 301 moves to detection reservoir 233a. The surface level of liquid in the reagent reservoir 301 is preferably kept higher than that of the detection reservoir 223a. Then, conveniently a reagent in the reagent reservoir 301 will flow towards the detection reservoir 223a, as soon as the liquid switch portion 257 on trigger channel 334a is opened.

Part of sample flowing along trigger channel 334a also enters trigger channel 335a, and, being further delayed during the channel through expanded channel region 263, reaches the liquid switch portion 257 on the trigger channel 235a. Then, the liquid switch portion 257 is opened, a coloring reagent stored in the reagent reservoir 302a will flow along the channel 333a to the detection reservoir 223a. The surface level of liquid in reagent reservoir 302a also is preferably kept higher than that of detection reservoir 223a.

Of enzyme-linked antibodies supplied to detection reservoir 223a, excess antibodies which did not bind to primary antibodies are pushed out with inflow of the coloring reagent solution towards dispensing channel 222. On the other hand, coloring reaction expected to occur at detection reservoir 223a takes a longer time than the removal of unreacted antibodies from detection reservoir 223a. Thus, it is possible to remove excess antibodies by using the coloring reagent without requiring deliberate use of cleaning buffer subsequent to the introduction of secondary antibodies. This reduces the necessary number of reagent reservoirs.

According to this embodiment, like the pretreatment unit 266 representing the fourth embodiment described above, the liquid levels of the detection reservoirs 223a to 223c, a series of reagent the reservoirs 301a to 301c, and another series of reagent the reservoirs 302a to 302c may adjusted as appropriate so that movement of reagents towards detection unit 214 smoothly occurs via capillary action. Through this arrangement, it is possible to simplify the structure of chip without requiring the use of an external driving unit.

According to the aforementioned configuration, plural reservoirs communicate with each other in detection unit 214, and the connections between reservoirs are altered via the open/closure of flow control units 314 provided to each connector channel. Therefore, it is possible to detect insulin dependent on the coloring reaction of sample by sequentially operation in the detection unit 214.

### Seventh Embodiment

The chips in the above embodiments will be beneficially used for biochemical test. Description will be given below about the chip on the premise that the chip is used for biochemical test in which the hepatic function is evaluated based on subject's blood sample.

The basic structure of the chip may be similar to that of the third embodiment. The detection unit 214 may include detection reservoirs 223 sufficiently to determine, for example, the test items listed in Table 2. The items listed in Table 2 can be determined via single step reactions by supplying in advance appropriate reagents to detection reservoirs 223. If a detection reservoir 223 is connected to a reagent reservoir, a flow control unit 314 attached to the channel connecting them should be closed.

Before using a chip for a test, the tester selects test items to be determined if necessary from the items listed in Table 2. If the tester wants to evaluate the hepatic function of a patient, he selects the items marked by open circle (‘∘’) in the column titled “Hepatic function set.” If the tester wants to evaluate the renal function of a patient, he selects the items marked by open circle (‘∘’) in the column titled “Renal function set.” In addition to aforementioned items, other items may be selected as needed. Then, the tester opens the flow control units 314 on the dispensing channels 222 leading to the detection reservoirs 223 assigned to the detection of selected items, while he closes the flow control units 314 on the dispensing channels 222 leading to the detection reservoirs 223 assigned to the detection of not-selected items. Thus, he can readily customize the configuration of the chip in accordance with the test items.

Table 2

TABLE 2

| | HEPATIC | RENAL | |
|---|---|---|---|
| ITEM | FUNCTION SET | FUNCTION SET | MEASUREMENT METHOD |
| TOTAL PROTEIN | | | BIURET METHOD |
| ALBUMIN | | | BCG METHOD |
| DIRECT-REACTING BILIRUBIN | | | ENZYME(BOX) METHOD |
| AST(ASPARTATE AMINOTRANSFER- | ◯ | | JSCC - BASED AST ASSAY |
| ASE) | | | |
| ALT(ALANINE AMINOTRANSFERASE) | ◯ | | IFCC METHOD |
| LDH(LACTODEHYDROGENASE) | ◯ | | CELLULOSE ACETATE MEMBRANE-EASED |
| | | | ELECTROPHORESIS & JSCC-BASEO LDH ASSAY |
| γ-GTP | ◯ | | JSCC-BASED γ-GTP ASSAY |
| ALP(ALKALINE PHOSPHATASE) | | | BESSEY-LOWRY METHOD |
| BUN(BLOOD UREA NITOROGEN) | | ◯ | UREASE/INDOPHENOL METHOD |
| CREATININE | | ◯ | ENZYME(POD)METHOD |
| AMYLASE | | | GS-CNP SUBSTRATE METHOD |
| TOTAL CHOLESTEROL | | | ENZYME (CHOLESTEROL ESTERASE OXIDASE(MET [symbol] |
| LDL-CHOLESTEROL | | | JSCC-BASED LDL-CHOLESTEROL ASSAY |

### Eighth Embodiment

In this embodiment, steps required for the execution of clinical biochemical test will be described with reference to the analysis portion described in relation to the sixth embodiment (FIGS. 50 to 53). The chip shown in FIGS. 50 to 53 has the detection unit 635 as analysis unit, but the following explanation will also apply to a chip having a measurement unit as analysis unit.

The chip is a general-purpose chip having an analysis unit as shown in FIGS. 50 to 53 which allows one to apply various detection reactions by opening/closing the flow control portions provided therein.

Reactions used for the clinical biochemical test can be classified according to the number of reaction steps into one-step reactions, two-step reactions and three-step reactions. Among typical test method used in the clinical biochemical test, colorimetry, enzyme method, UV method, latex agglutination method (LA method), latex agglutination turbidity immunoassay (LATIA assay), turbidity immunoassay (TIA assay), and selective inhibition belong basically to one-step reactions. Even if an pretreatment step is involved, they can complete in two steps. Radio immunoassay (RIA), chemical luminescence assay (CLIA), chemical luminescence enzyme-linked immunoassay (CLEIA), enzyme-linked immunosorbent immunoassay (ELISA) can basically complete in three steps.

One-step to three-step reactions will be described below with reference to the analysis portion shown in FIGS. 50 to 53. However, four or more or multi-step reactions will be achieved in the same manner as in one-step to three-step reactions using the present chip, by only increasing the number of the reservoirs and the liquid switch portions provided to detection unit 635.

Practice of one-step reaction will be described first. One-step reaction is a reaction obtained by mixing a sample with a reagent directly. One-step reaction is allowed to occur at detection unit 635 (such type of detection unit is called as class 1 detection unit) shown in FIG. 50 where flow control unit 601 is closed. For one-step reaction, reservoir 630 is used for detection reservoir. A reagent necessary for the reaction is introduced in advance in reservoir 630 in accordance with a kind of a substance to be detected and the measurement method. After the reagent is applied, a lid 700 may be joined to the chip.

The reagent may include coloring reagent which react with the substance to be detected to give a color or dye for coloring reaction as in the quantification of albumin. As for the enzyme method, the reagent may be an enzyme which use a substance to be detected to give a color, and the like. As for the UV method, the reagent may be substrate, or co-enzyme (NAD⁺/NADH or NADP⁺/NADPH) for the reaction of the enzyme being a substance to be detected. As for the latex agglutination method or the latex agglutination turbidity immunoassay, the reagent may be suspension of latex beads on which antibodies against a substance to be detected are immobilized. As for the turbidity immunoassay, the reagent may be antibody solution, the antibody being against a substance to be detected. These reagents are set at the appropriate volume ratio to the sample. A suitable reagent for a given reaction may be determined by referring to a textbook (for example, see Kanai, I. (author), Kanai, M. (ed.), “Handbook of Clinical Laboratory Test,” Revised 31st Edition, Kanehara Publishing).

When sample flows along main channel 221 and reaches the branching portion of channel 607 connected to reservoir 630, it reaches reservoir 630 to fill it since flow control portion 600 is open. However, since the channel 607 connecting reservoir 630 to reservoir 631 is closed at liquid switch portion 623, entry of sample to reservoir 630 is stopped when sample fills reservoir 630. A bulged body 642 can be made of the material which close down the channel after the reservoir 630 is sufficiently filled, so as to prevent the countercurrent of sample to the main channel 221 during reaction. Sample encounters reagent in reservoir 630 to evoke detection reaction. The smaller the capacity of reservoir 630 is, the faster the sample intermingles with reagent. The other part of sample advances further along main channel 221, but cannot enter trigger channel 620 because flow control unit 601 is closed. Thus, liquid switch portion 623 is kept closed.

When the reaction involves coloration or enzyme reaction, sample is exposed to reagent for a specified time. Then, the absorption in reservoir 630 is measured, by using the reservoir 630 as an optical cell. For example, a light is irradiated with over lid 700 and the transmitted light is received by a photosensitive unit placed beneath substrate 701. As for UV method, determination of the consumption of coenzyme (NAD⁺/NADH or NADP⁺/NADPH) by UV (ultraviolet ray) proceeds in the same manner. The absorption of UV by the reaction system is followed at regular intervals, and the activity of enzyme to be detected is determined based on the consumption speed of enzyme. When UV method is used, substrate 700 and lid 700 are preferably made of quartz because it transports UV light well.

When latex agglutination (LA) method is used, latex beads are allowed to mix with sample, and left to deposit on the bottom of reaction reserboir. Then, the absorption of light in the reservoir 630 is measured. For latex agglutination method, the reservoir 630 preferably has a conical or semi-circular bottom. If sample does not contain any reactive agent, individual beads will not agglutinate but simply precipitate as separate particles and concentrate to the summit of cone or semi-circle, which enhances the transmission of light. If sample contains reactive agent, beads agglutinate together to form aggregates that adhere to the bottom surface. Thus, beads spread over the bottom that lowers the transmission of light. Thus, it is possible to determine whether sample contains a target reactive substance or not by measuring the transmittance of reservoir 630.

When latex agglutination turbidity immunoassay (LATIA) is used, the turbidity of reaction system is measured at regular intervals, without waiting to occur precipitation, thereby monitoring how fast the turbidity increases as a result of agglutination. Based on the result, it is possible to quantify a target substance. According to turbidity immunoassay (TIA), change of the turbidity due to aggregates of substances in a sample and the antibody is measured.

Next, description will be given about how two-step reaction is allowed to occur at reaction portion. Two-step reaction is used for a step for pre-treating a substance that is used for reagent for the later one-step reaction. For two-step reaction, a detection unit 635 (such type detection unit will be called as class 2 detection unit hereinafter) is employed in which, according to FIG. 50, flow control portions 600 and 601 are opened, and flow control portion 602 is closed. With class 2 detection unit 635, reservoir 631 instead of reservoir 630 is used for measurement. Into reservoir 631 is introduced a reagent as used in one-step reaction. Into reservoir 630 is introduced a reagent necessary for pretreatment: if pretreatment involves the prohibition of re-coagulation, at least one chosen from heparin, EDTA and citric acid in the form of dry powder is introduced. The reservoir 633 is kept empty.

The procedures necessary for allowing sample to enter via flow control portion 600 to reservoir 630 are similar to those observed in class 1 detection unit. With class 2 detection unit, since flow control unit 601 is open, a sample passes along lag channel 610 and advances through trigger channels 620 and 621 to reach the liquid switch portion 623 and open it. The delay time determined by lag channel 610 is so chosen as to allow sample and reagent to be mixed well in reservoir 630. When liquid switch portion 623 becomes open, communication between reservoir 630 and 631 is established, sample having undergone pretreatment enters reservoir 631 via capillary action or supported by water-level difference. There, sample intermingles with reagent at reservoir 631. Since flow control portion 602 is closed, liquid switch portion 624 is not opened. Liquid flowing into reservoir 631 stays there. The reaction in reservoir 631 is subjected to measurement.

Next, description will be given about how three-step reaction is allowed to occur at reaction portion. For three-step reaction, a detection unit 635 (such type detection unit 635 will be called as class 3 detection portion hereinafter) is employed in which, according to FIG. 50, all flow control portions 600, 601, 602 are opened.

When class 3 reaction unit is used for immunological reaction detected by radio immunoassay (RIA), an antibody against a target substance is immobilized in advance to the inner surface of reservoir 630. Into reservoir 633, a radio-labeled standard solution is introduced, while into reservoir 634, an emulsified scintillation solution which transforms radioactive energy into light energy is introduced. Immobilization of antibody to the surface of reservoir 630 may be achieved physically by spontaneous adsorption to the clean surface of the material, or chemically by using a coupling agent having amino or carboxyl group.

The procedures necessary for allowing sample to enter from main channel 221 to reach reservoir 630 are similar to those observed in class 1 detection portion. Then, a target substance in sample bind to antibody immobilized on the surface of reservoir 630. In contrast with class 1 reaction unit, however, since flow control portions 601 and 602 are open, part of sample passes along lag channel 610 and trigger channels 620 and 621 to open liquid switch portions 623, 625 sequentially. Thus, communication is established between reservoir 630 and reservoir 631, and part of sample is evacuated into reservoir 631 that is used as waste reservoir. The delay time determined by lag channel 610 is so chosen as to allow sample and antibody to sufficiently interact each other in reservoir 630. When liquid switch portion 625 is opened, radioactive standard solution stored in reservoir 633 passes via channel 607 to reservoir 630, and pushes out sample staying at reservoir 630 towards reservoir 631, and fills reservoir 630. These sequential flows terminate when reservoir 631 is filled. At this stage, in reservoir 630, radioactive standard substance in solution and a specific substance in sample compete for binding to antibody. The more the content of specific substance in sample is, the less the radioactive standard substance bound to antibody is.

Sample, passing along lag channel 612, opens liquid switch portions 624, 626 sequentially, liquid in reservoir 631 flows towards reservoir 632 while emulsified scintillator solution stored in reservoir 634 passes along channel 607 and pushes liquid in reservoir 630 towards reservoir 631, 632. The delay time determined by lag channel 611 is so chosen as to allow reaction between specific substance in sample and antibody to reach equilibrium.

These sequential flows terminate when reservoir 631 is filled. At this stage, reservoir 630 is filled with emulsified scintillator solution. The less the content of specific substance in sample is, the more the radioactive standard substance bound to antibody is. The emulsified scintillator solution filling reservoir 630 emits light in the darkness, being activated by the radioactive standard substance. The energy of light is counted by a photo-counter. Based on the result, it is possible to estimate the content of the specific substance in the sample.

When class 3 detection unit 635 is used for immunological reaction detected by chemical luminescence immunoassay (CLIA), an antibody specific for a target substance is immobilized in advance to the inner surface of reservoir 630. Into reservoir 633, a solution with luminescent antibody is introduced. The luminescent antibody is obtained by attaching chemical luminescent substance (acridinium ester or the like) to the antibody specific to a target substance. Into reservoir 634, buffer for washing is introduced.

Sample is allowed to fill reservoir 630 via the same procedures as for one-step reaction, and react with antibody there until liquid switch portions 623 and 625 are sequentially opened. The delay time determined by lag channel 610 is so chosen as to allow a target substance and antibody to react fully. As soon as liquid switch portion 625 is opened, luminescent antibody solution stored in reservoir 633 enters via channel 607 into reservoir 630 to wash out liquid there towards reservoir 631. The inflow of luminescent antibody solution terminates filling reservoir 630 when reservoir 631 is filled with the solution.

Sample passes along lag channel 612, and liquid switch portions 624, 626 are opened sequentially. Then, liquid in reservoir 631 flows towards reservoir 632, and then washing buffer stored in reservoir 634 flows along channel 607 and washes out the content of reservoir 630 towards reservoir 632. The delay time determined by lag channel 611 is so chosen as to allow a target substance and luminescent antibody to bind together fully. These sequential flows terminate when reservoir 632 is filled. At this stage, reservoir 630 is filled with washing buffer. The higher the concentration of specific substance in sample in reservoir 630 is, the more the luminescent antibody binds to the substance. Thus, it is possible to estimate the concentration of the specific substance in sample by measuring the intensity of luminescence.

Chemical luminescence enzyme-linked immunoassay (CLEIA) can be also employed by using class 3 detection unit 635 according to the same procedures used in chemical luminescence immunoassay (CLIA). Instead of antibody specific to a target substance coupled with chemical luminescent substrate, antibody against the target substance and coupled with an enzyme that react with luminescent substrate to develop color is set in reservoir 633, and instead of washing buffer, luminescent substrate solution is set in reservoir 633, so the measurement can be employed as the same procedures used in CLIA.

When class 3 detection unit 635 is used for immunological reaction detected by enzyme immunoassay (EIA), the same procedures as used in chemical luminescence immunoassay (CLISA) may be employed. Instead of antibody against the target substance and coupled with an enzyme that react with luminescent substrate, a solution of an antibody against the target substance and to which an enzyme such as peroxidase is linked is prepared wherein the enzyme will develop color as a result of reaction with its substrate. Into reservoir 634, a die solution responsible for the development of color instead of substrate solution is introduced. The remaining procedures are the same with those for CLEIA.

For the three-step reactions described above, washing occurs at single step. However, the more the number of washing is, the higher the precision of measurement is. Thus, if it is required to improve the precision of measurement by increasing multiple-step washing, it is only necessary to add a desired number of sets of reservoir, lag channels, trigger channels, and liquid switch portions.

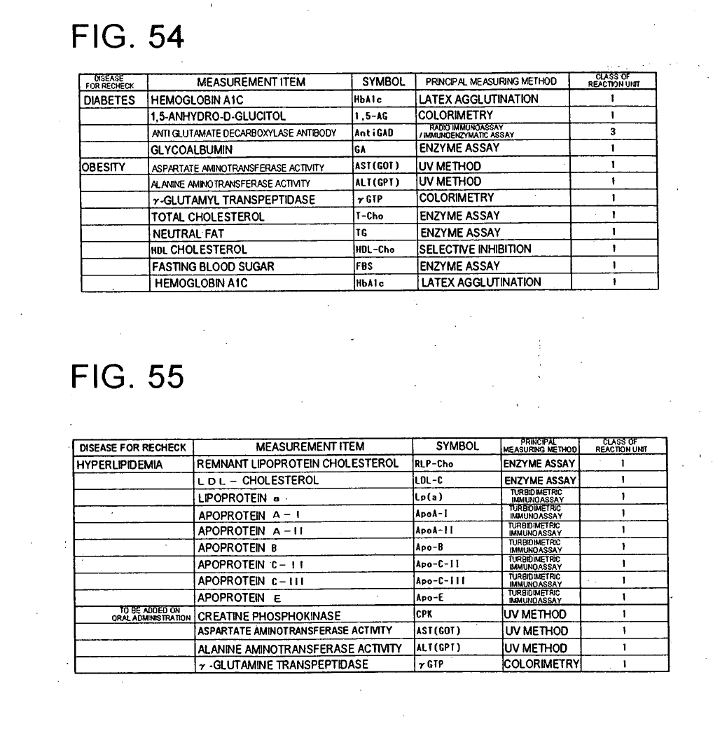

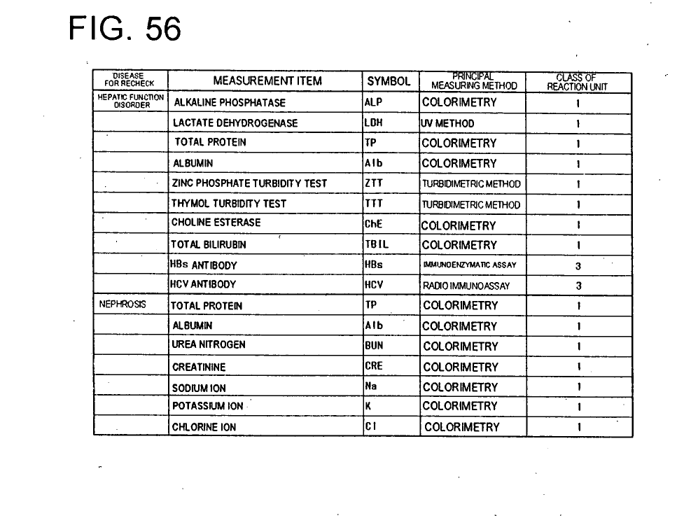

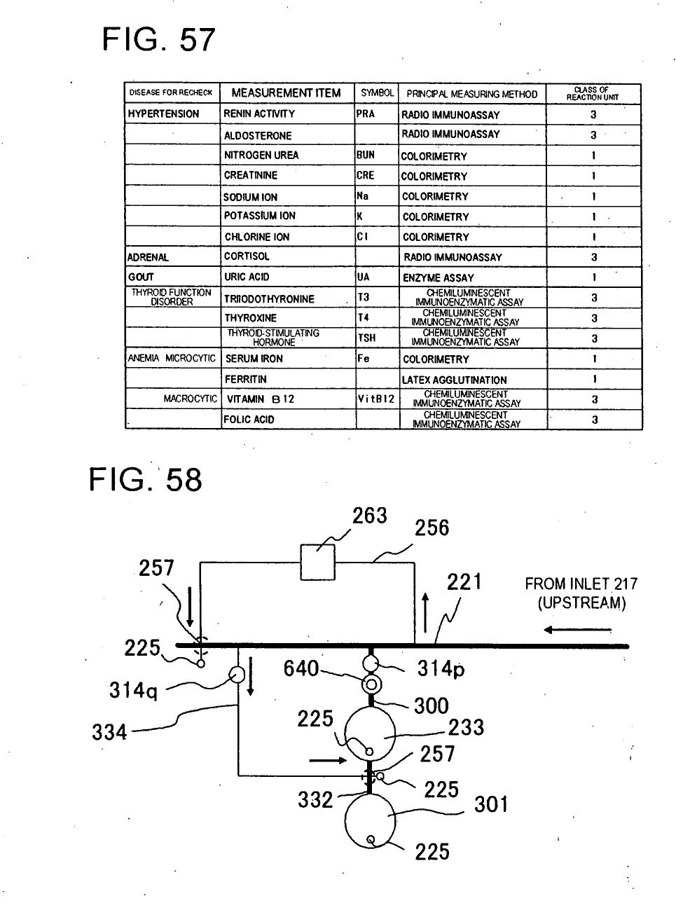

FIGS. 54 to 57 cite lists of principal test items required for an ordinary recheck test, involved methods, and applicable class of reaction unit. From the inspection of FIGS. 54 to 57, it is obvious that a chip containing class 1, 2 and 3 reaction units can well manage common recheck tests. Namely, it is possible to prepare in advance standardized general-purpose chips which can manage ordinary test items required for a recheck test shown in FIGS. 54 to 57.

For example, based on the list shown in FIG. 54, it is possible to prepare a general-purpose chip for the diabetes which includes one or more of the class 1 reaction unit and one of the class 3 reaction unit. With such a chip at hand, it is possible to check the condition of diabetes readily at site. Prior to the test, however, it is necessary to introduce at least one reagent necessary for checking diabetes is introduced into at least one reaction unit. As for the class 1 reaction unit, for example, at least one chosen from hemoglobin A1c, 1,5-anhydro-D-glucitol, and glycoalbumin should be introduced. If it is required to know the activity of enzyme in diabetes, a reagent necessary for assaying anti glutamate decarboxylase antibody should be introduced into a class 3 reaction unit. The reaction units of a chip are not necessarily filled with reagents.

With this general purpose chip used for the check of diabetes, it is possible to readily select the appropriate items at site according to the disease condition or history of a patient to be examined. According to the method of the embodiment, it is thus possible by using a general-purpose chip to customize its reaction unit at a post-processing stage to match an individual need.

As shown in FIGS. 54 to 57, general-purpose chips for the diagnosis of obesity, hyperlipidemia, hepatic function disorde, nephrosis, hypertension, adrenal function, gout, thyroid function disorder, anemia (microcytic, macrocytic) can be prepared. For the diagnosis of a disease as mentioned above, it is possible as in diabetes to modify the configuration of a chip according to the disease, and to supply reagents for items described in FIGS. 54 to 57 as appropriate to its reaction units of the appropriate class.

The general-purpose chip may be so constructed as to have the same number of analysis units with that used for sample measurement, and may be subjected to the same analysis task using a standard solution instead of the sample. Thus, the measurement further enhanced in the accuracy can be realized based on the general-purpose chip.

### Ninth Embodiment

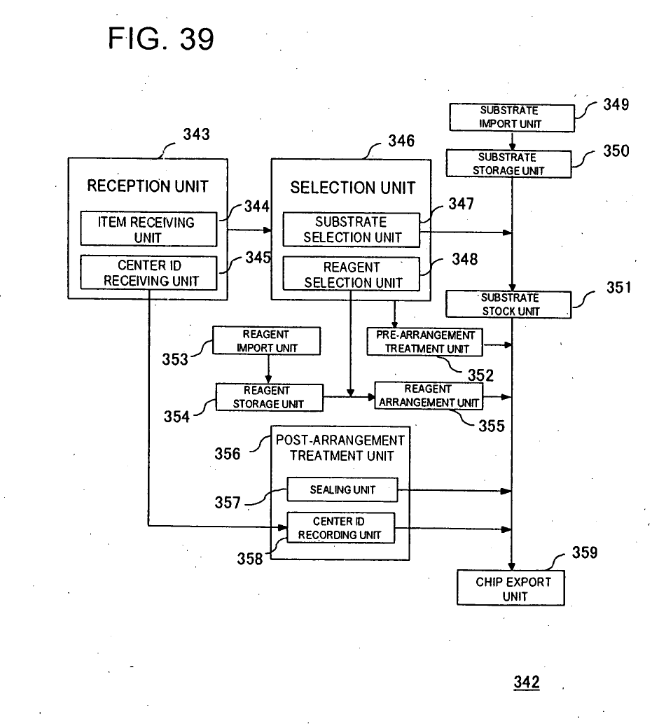

The chip represented by the above embodiments may be manufactured with a machine as described below. FIG. 39 shows a schematic diagram for showing an exemplary chip manufacturing system representing this embodiment. The chip manufacturing apparatus 342 shown in FIG. 39 is an apparatus for producing a chip whose configuration is customized according to a request from a laboratory center. Description will be given below of the system on the premise that the chip manufactured has a detection unit 214 as analysis portion. However, the present system can be applied as well for the manufacture of a chip having a measurement unit 233 as analysis portion.

The chip manufacturing apparatus 342 includes a reception unit 343, selecting unit 346, chip import unit 349, chip storage unit 350, chip stock unit 351, pre-arrangement treatment unit 352, reagent import unit 353, reagent storage unit 354, reagent arrangement unit 355, post-arrangement treatment unit 356, and chip export unit 359.

A substrate 216 is transported via chip import unit 349 to chip storage unit 350 where the substrate 216 is stored. Reagents and buffer necessary for reactions occurring at detection reservoirs 223 are transported via reagent import unit 353 to reagent storage unit 354 where they are stored. The reagent may be stored in the form of beads on which it is carried.

Reception section 343 receives input from a laboratory center that will use a chip. Reception section 343 includes item receiving unit 344 and center ID receiving unit 345. Parameter receiving subsection 344 receives input about information of parameters to be determined with a chip. Center ID receiving unit 345 receives IDs of a test center or a doctor, who are the client for the chip manufacturer.

Selection unit 346 selects a substrate 216 and detection reagents based on the information provided by reception unit 343. Substrate selection unit 347 selects a substrate 216 used for the fabrication of a chip. The selected substrate 216 is transported from substrate storage unit 350 to substrate stock unit 351. Reagent selection unit 348 selects reagents including detection reagents and buffers to be introduced into detection reservoir 223, reagent reservoir 301, reagent reservoir 302, and other reservoirs. The thus selected reagents are transported from reagent storage unit 354 to reagent arrangement unit 355.

Pre-arrangement treatment unit 352 activates the surface of substrate 216 selected by selecting unit 346 according to the information inputted in the reception unit 343 so that a selected reagent efficiently adsorbs to the surface of substrate 216. The same section may apply a cover over the substrate for fear that reagents may be blown off from the region to filled with the reagent.

Reagent arrangement unit 355 distributes reagents including detection reagents or buffers selected according to required items to detection reservoir 223, reagent reservoir 301, detection reservoir 302 and other reservoirs of substrate 216 held at substrate stock unit 351. If distribution of liquid reagent is required, a certain amount of the reagent may be transferred into a cylinder, and part or all of the cylinder content may be injected to an assigned area. The injected reagent may be exposed to dry air or nitrogen gas to be dried and solidified being deprived of the solvent. If reagent beads are used, a bead having a sufficient size to carry a sufficient amount of reagent as to cause detectable reaction in detection reservoir 223 may be prepared in advance, and such a bead may be supplied to detection reservoir 223.

Post-arrangement treatment unit 356 opens or closes flow control units 314 as appropriate based on the information about the necessary items of a test provided by item receiving unit 344. Post-arrangement treatment unit 356 has sealing unit 357 and center ID recording unit 358. Sealing unit 357 applies a seal 227 on the surface of substrate 216, thereby protecting the surface of substrate 216. Sealing unit 357 may further seal channels, detection reservoirs 223, fraction portion 235 or other reservoirs selected for the purpose. Center ID recording unit 358 records the ID of centers that provide information to center ID receiving unit 345. The ID may be recorded on substrate 216, or printed on a package of substrate 216.

Substrate stock unit 351 transports the thus prepared chip to chip export unit 359. The chip may be packed with an air-tight packaging material as needed, and the pack may be filled with inert gas such as nitrogen. The packaging material may be sealed.

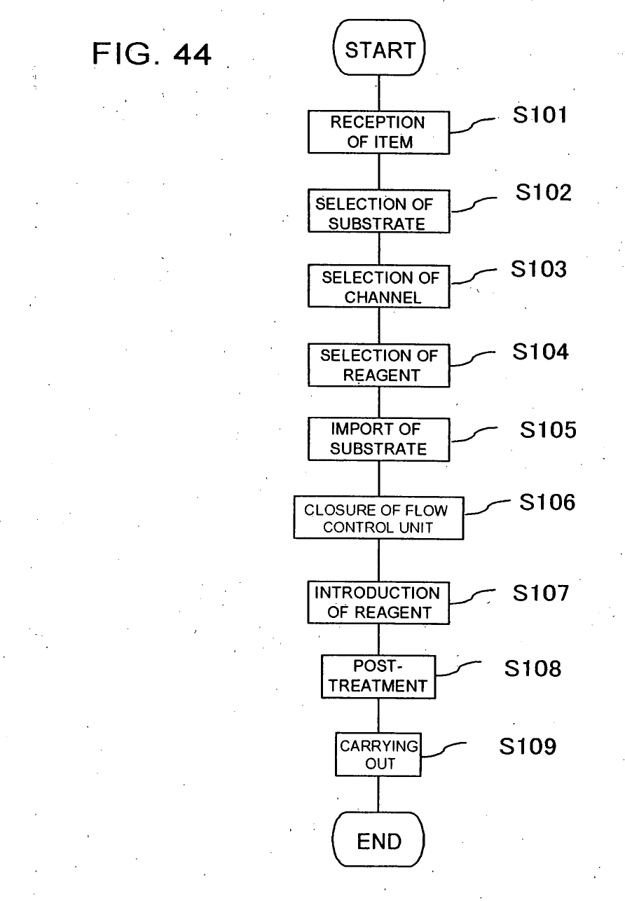

FIG. 44 shows the procedures for the manufacture of a chip using a chip manufacturing apparatus shown in FIG. 39. Referring to FIG. 44, reception unit 345 receives input carrying the information about the ID of clinical center and necessary items (S101). Substrate selection subsection 347 selects a substrate according to the information (S102), and selects channels and channels to be activated on the substrate (S103). Reagent selection subsection 348 selects reagents according to the required parameters (S104). The selected substrate is imported (S105). Pre-arrangement treatment section 352 opens/closes flow control portions as appropriate so that sample can flow along selected channels (S106). Reagent arrangement unit 355 introduces required reagents into their assigned sites on the substrate (S107). Then, post-arrangement treatment section performs post-treatment step (S108). The resulting chip is exported (S109).

In the above procedures, selection of a substrate at step 102 and selection of reagents at step 104 may be exchanged. Alternatively, selection of a substrate at step 102 may be followed by the renewed import of another substrate, and then by selection of reagents at step 104.

By using the chip manufacturing apparatus 342, it is possible to readily produce a chip whose configuration is customized in accordance with the items provided to reception unit 343. Therefore, it is possible to provide chips whose configuration is optimized for individual needs, even if the number of chip users is immense.

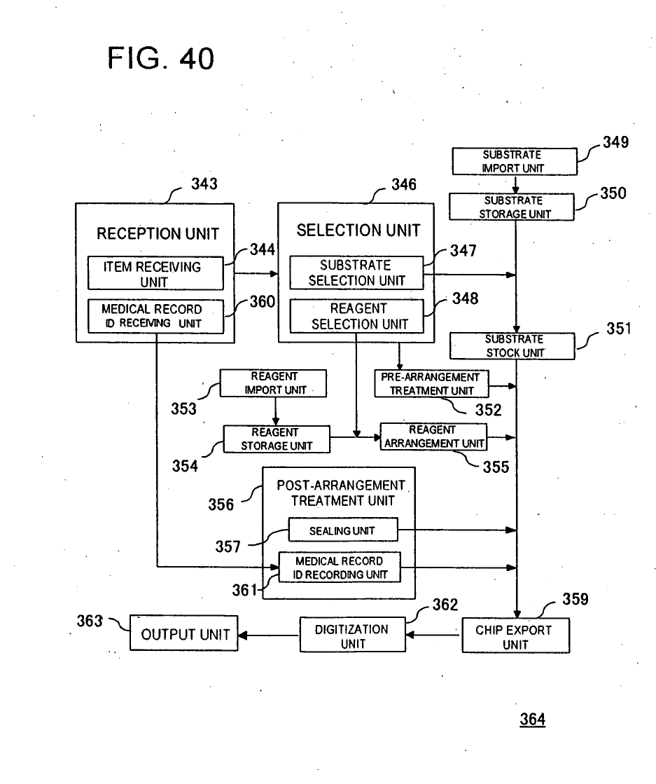

FIG. 40 shows a schematic diagram for showing an exemplary chip manufacturing system capable of producing chips whose configuration can be customized according to the health conditions of patients who have received clinical tests in a laboratory of a clinical center. The chip manufacturing system 364 shown in FIG. 40 is basically similar to the system 342 shown in FIG. 39, except that the former has medical record ID reception unit 360 instead of center ID reception unit 345, and medical record ID recording unit 361 instead of center ID recording unit 358, and that the former additionally includes digitization unit 362 and output delivery unit 363.

Medical record reception subsection 360 receives input about the information of ID data of the patients of hospitals and visitors for receiving laboratory tests. Medical record recording unit 361 records or prints the ID of a patient on a chip or its package.

Digitization unit 362 converts the test result obtained via a chip into numerical data, and delivers the data to output delivery unit 363. Output delivery unit 363 converts the numerical data into graphs and presents the graphs on display.

According to the chip manufacturing system 364, since each chip has the ID of a patient printed thereon, the doctor and the other person engaged in the test can securely identify which patient a given chip represents. Moreover, since digitization unit 362 converts the test result into corresponding numerical data, it is quite easy to add the data to an electronic medical record. Digitization unit 362 may have the same configuration with that of measuring apparatus 237 described above in relation to the second embodiment.

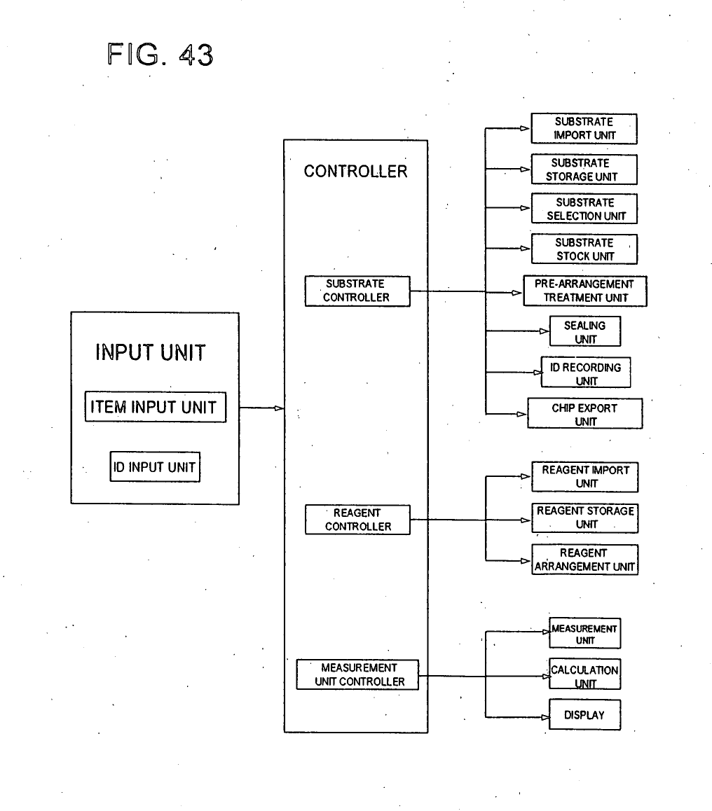

For the chip manufacturing system shown in FIG. 39 or 40, control of hardware is achieved by a controller. FIG. 43 shows the organization of such a chip manufacturing apparatus.

Referring to FIG. 43, input unit corresponds to reception unit of the system shown in FIG. 39 or 40, and includes test item input unit and ID input unit. The controller includes a substrate controller, a reagent controller, and a measurement portion controller.

Substrate controller controls, based on the information provided by input unit, the selection of substrate and active channels, and operation involved in the import of a selected substrate and its export. The reagent controller controls, based on the information provided by input unit, selection of reagents, and distribution of reagents to specified sites. The measurement portion controller controls, if the apparatus includes a measurement unit in itself, the measurement unit, a calculation unit responsible for the computation of measurement results, and a display for displaying measurement results.

Description has been given above on the premise that a chip is manufactured in a chip factory in which a chip is manufactured on receipt of an order. However, the chip may be customized in a clinical center. For example, an operator in the clinical center can customize a chip at the test stage, by opening/closing flow control units.

The present invention has been described above with reference to the embodiments. These embodiments are presented just for illustration, and various modifications and variations thereof are easily thinkable. It will be quite obvious for those skilled in the art that those modifications and variations are also included within the scope of the invention.

For example, according to the above embodiments, the flow control portions are provided to some channels, and a selected flow control portion(s) is closed, thereby determining flow through other channels. However, the flow control portions which can be opened may be provided to channels. In this case, initially all flow control potions are closed, and after appropriate channels are selected according to the kind of sample or treatment, the flow control portions connected to those channel are selected and are opened so that sample can be guided through desired channels. With the above arrangement, it is still possible to customize a chip according to a kind of the sample or test items.

A flow control unit 314 which is initially closed but can be opened later at a desired time may be manufactured by the following method. The flow control unit 314 has its surface hydrophobic at first which leads to the closure of the portion. Later at a desired time, UV light is impinged to the portion to open it. The hydrophobic treatment may be achieved by using silane coupling agent, silicone oil, and the like, or by forming a thin film of PDMS. The material of the organic film is oxidized and decomposed, when exposed to UV light, to turn hydrophilic compound. Thus, at first, the surface of a flow control portion in contact with a channel is treated with an agent as described above to be hydrophobic or water-repellent, then a UV beam concentrated by using a lens system is impinged onto the flow control unit 314 to be opened. Then, the flow control unit 314 is opened.

An alternative method uses an organic substance having a low boiling point for closure and IR laser radiation for opening. A suitable substance having a low boiling point includes paraffin. When paraffin is used, a substrate 216 is heated to a temperature close to the melting point of paraffin. Paraffin in the form of a thin rod is contacted to a flow control unit 314 in a short time, thereby attaching softened paraffin to the channel surface. Thus, the flow control unit 314 is closed. Later at a desired time, IR laser beam is irradiated to the flow control unit 314. Since paraffin absorbs IR ray, it is heated above its melting-point so that it is melted and vaporized. If paraffin is completely eliminated, the flow control unit 314 is opened.

In the above embodiments, sample introduction unit 212 includes the single inlet 217. However, sample introduction unit may comprise plural inlets 217. By providing the plurality of the inlet 217, it is possible to perform multiple tests using a single chip, for example, perform tests on different samples collected from a single patient such as blood, saliva, urine, nasal secretion, and the like. It is also possible to perform multiple tests in parallel using a single chip, for example, perform parallel tests on multiple samples of a single kind (for example, blood) collected from many patients. For a chip having plural inlets 217, it is also possible to insert a flow control unit 314 between sample introduction unit 212 and separation unit 213. Through this arrangement, it is possible to select the passage of a sample according to the necessity of the separation unit 213.

According to the above embodiments, flow control units 314 are provided to all dispensing channels 222. However, flow control units 314 may be provided to some of dispensing channels 222. For example, if a chip has the detection reservoir 223, the flow control unit 314 may be provided except the detection reservoir which is utilized in every test.

The above embodiments have been illustrated on the premise that the general shape of the detection reservoirs and the fraction portions is cylindrical. However, the general shape thererof is not limited to cylinder but may take any desired shape, as long as it can be used for the analysis (detection or measurement) of the content therein. The shape of the detection reservoirs or the fraction portions may be rectangular column such as a quadrangular prism. The reservoirs and fraction portions may not be a diverticular-like shape, but a channel-like shape, as has already been noted referring to FIG. 9.

What is said above applies not only to the detection reservoirs and the fraction portions but also to other reservoirs. Namely, those reservoirs may take a shape other than the cylinder, as long as the shape ensures a sufficient volume to hold a liquid which is introduced or corrected thereinto For example, the shape of the reservoirs formed on the chip may be a rectangular column or the channel-like shape having the predetermined plane shape. The reservoir which function as a waste reservoir may also take, for example, a form like a zigzag line, like cylinder with protrusions and cavities on its inner surface. A waste reservoir with such surface contour, because of the increase of surface area, can further enhance capillary action, and ensure the reliable recovery of a waste liquid.

1. A chip comprising:
a substrate;
plural channels formed on the substrate; and
flow control portions provided to any one of said plural channels, each of the flow control portions capable of being closed, wherein:
closure of the flow control portion of a channel among said plural channels allows a sample to be guided to other channel.

2. The chip according to claim 1, wherein:
at least part of said flow control portion is made of a thermoplastic material; and
said flow control portion, when it is closed, is deformed by being heated above the temperature of the glass transition temperature of said thermoplastic material to clog and occlude the channel.

3. The chip according to claim 1, wherein:
said flow control portion, when it is closed, has a hydrophobic surface made by stamping at least a part of the channel with a silicone resin containing polydimethyl siloxane.

4. The chip according to claim 1, further comprising:
a damming portion for damming a liquid in the channel;
a trigger channel in communication with the channel close to said damming portion, said trigger channel being for guiding said liquid to said damming portion, wherein:
the chip has a liquid switch portion for controlling the flow of a liquid through the channel.

5. The chip according to claim 1, further comprising:
a sample introduction portion formed on the substrate;
an analysis portion for analyzing a specified component contained in a sample introduced via said sample introduction portion; and
a plurality of said channels connecting said sample introduction portion with said analysis portion; wherein:
closure of the flow control portion provided to a channel among said plurality of said channels allows said sample to be guided via other channel to said analysis portion.

6. The chip according to claim 1, further comprising:
a sample introduction portion formed on the substrate;
an analysis portion for analyzing a specified component contained in a sample introduced via said sample introduction portion; and
branched channels for guiding said sample introduced via said sample introduction portion to a plurality of said analysis portions, wherein:
closure of the flow control portion provided to said channel branched towards one of the analysis portions allows the sample to be guided to the other analysis portion.

7. The chip according to claim 5, further comprising a separation portion including a part of the channel which separates a component contained in said sample introduced via the sample introduction portion from the sample to guide the component to the analysis portion.

8. The chip according to claim 6, further comprising a separation portion including a part of the channel which separates a component contained in said sample introduced via the sample introduction portion from the sample to guide the component to the analysis portion.

9. The chip according to claim 5, further comprising a pretreatment portion which applies specified pretreatment on the sample introduced via the sample introduction portion.

10. The chip according to claim 6, further comprising a pretreatment portion which applies specified pretreatment on the sample introduced via the sample introduction portion.

11. The chip according to claim 9, further comprising a separation portion including a part of the channel which separates a component contained in said sample introduced via the sample introduction portion from the sample to guide the component to the analysis portion, wherein:
said pretreatment portion comprises a reservoir, and a liquid switch portion provided downstream of the reservoir which controls the flow of a liquid sample from said pretreatment portion to said separation portion; and
said liquid switch portion comprises a damming portion for damming a liquid in the reservoir, and a trigger channel which communicates with the channel close to said damming portion, and guides the liquid to said damming portion, and wherein the flow control portion is provided to said trigger channel.

12. The chip according to claim 10, further comprising a separation portion including a part of the channel which separates a component contained in said sample introduced via the sample introduction portion from the sample to guide the component to the analysis portion, wherein:
said pretreatment portion comprises a reservoir, and a liquid switch portion provided downstream of the reservoir which controls the flow of a liquid sample from said pretreatment portion to said separation portion; and
said liquid switch portion comprises a damming portion for damming a liquid in the reservoir, and a trigger channel which communicates with the channel close to said damming portion, and guides the liquid to said damming portion, and wherein the flow control portion is provided to said trigger channel.

13. The chip according to claim 7, further comprising a reaction portion where a component separated at said separation portion undergoes a specified reaction.

14. The chip according to claim 8, further comprising a reaction portion where a component separated at said separation portion undergoes a specified reaction.

15. The chip according to claim 13, wherein:
said reaction portion comprises a reservoir and a liquid switch portion provided downstream of said reservoir; and
said liquid switch portion comprises a damming portion for damming a liquid in said reservoir, and a trigger channel which communicates with a channel close to said damming portion, and guides the liquid to said damming portion, and wherein the flow control portion is provided to said trigger channel.

16. The chip according to claim 14, wherein:
said reaction portion comprises a reservoir and a liquid switch portion provided downstream of said reservoir; and
said liquid switch portion comprises a damming portion for damming a liquid in said reservoir, and a trigger channel which communicates with a channel close to said damming portion, and guides the liquid to said damming portion, and wherein the flow control portion is provided to said trigger channel.

17. The chip according to claim 1, further comprising a member capable of recording an identifying symbol of a medical record.

18. A method for producing a chip comprising:
preparing a substrate in which plural channels are formed; and
closing some of the channels by heating under pressure those channels to thereby cause deformation.

19. The method according to claim 18, further comprising:
providing a liquid switch portion by forming a damming portion for damming a liquid in the channel, and forming a trigger channel in communication with said channel close to said damming portion, said trigger channel being for guiding said liquid to said damming portion.

20. The method according to claim 18, further comprising:
preparing a member capable of recording an identifying symbol of a medical record; and
writing an identifying symbol of a medical record on the member.

21. The method according to claim 18, further comprising:
forming a reaction portion or a reservoir where a component in a sample is allowed to undergo a specified reaction;
introducing a reagent or a reagent solution into said reaction portion or said reservoir; and
covering the substrate after said introducing the reagent or the reagent solution.

22. An analysis portion comprising:
a main channel;
a reservoir;
a channel connecting said main channel and said reservoir;
a damming portion provided to the channel for damming a liquid in said reservoir;
a trigger channel in communication with said channel close to said damming portion, said trigger channel being for guiding the liquid to said damming portion;
a liquid switch portion comprising said damming portion and said trigger channel;
a lag channel provided to said trigger channel or said channel; and
a flow control portion for setting an opening and closing of said channel or said trigger channel.

23. The analysis portion according to claim 22, further comprising a closing switch for closing said channel.

24. The analysis portion according to claim 22, wherein said reservoir holds a reagent.

25. The analysis portion according to claim 23, comprising:
the two reservoirs;
the one liquid switch portion;
the one closing switch;
the one lag channel; and
the one or two flow control portions.

26. The analysis portion according to claim 23, comprising:
the five reservoirs; and
the two or more liquid switch portions, the two or more closing switches, the two or more lag channels, and the two or more flow control portions.

27. A chip comprising a substrate and the analysis portion according to claim 22, the analysis portion being formed on said substrate.

28. A chip having an analysis portion comprising:
a first treatment portion comprising:
a main channel;
at least one reservoir;
a channel connecting said reservoir and said main channel; and
at least one flow control portion for controlling an opening and closing of said channel, and
a second treatment portion comprising:
a main channel;
at least five reservoirs;
a channel connecting said reservoirs and said main channel;
a damming portion provided to said channel for damming a liquid in said reservoir;
a trigger channel in communication with said channel close to said damming portion, said trigger channel being for guiding said liquid to said damming portion;
at least four liquid switch portions each comprising said damming portion and said trigger channel;
at least one closing switch for closing said channel;
at least two lag channels provided to said trigger channel or said channel; and
at least two flow control portions for setting an opening and closing of said channel or said trigger channel, wherein:
the analysis portion comprises:
at least the three first treatment portions; and
at least the one second treatment portion, with either one of the first and the second treatment portions having at least the one reservoir holding a reagent, wherein:
when said first treatment portion has the reagent,
the reagent is necessary for determining any one, or two or more test items selected from the group consisting of:
hemoglobin A1c;
1,5-anhydro-D-glucitol; and
glycoalbumin, and
when said second treatment portion has the reagent, the reagent is necessary for detecting anti glutamate decarboxylase antibody.

29. A chip having an analysis portion comprising:
at least eight first treatment portions each of which includes:
a main channel;
at least one reservoir;
a channel connecting said main channel and said reservoir;
at least one flow control portion for setting an opening and closuring of said channel,
wherein at least one of the eight first treatment portions has at least one reservoir in which reagent has been stored,
the reagent being necessary for determining any one, or two or more test items selected from the group consisting of:
aspartate aminotransferase;
alanine aminotransferase;
γ-glutamyl transpeptidase;
total cholesterol;
neutral fatty acid;
HDL cholesterol;
fasting blood sugar; and
hemoglobin A1c.

30. A chip having an analysis portion comprising:
at least nine first treatment portions each of which includes:
a main channel;
at least one reservoir;
a channel connecting said main channel and said reservoir;
at least one flow control portion for setting an opening and closing of said channel,
wherein at least one of the nine first treatment portions has at least one reservoir in which reagent has been stored,
the reagent being necessary for determining any one, or two or more test items selected from the group consisting of:
remnant lipoprotein cholesterol;
LDL-cholesterol;
lipoprotein a;
apoprotein A-I;
apoprotein A-II;
apoprotein B;
apoprotein C-II;
apoprotein C-III;
apoprotein E;
creatine phosphokinase;
aspartate aminotransferase;
alanine aminotransferase; and
γ-glutamine transpeptidase.

31. A chip having an analysis portion comprising:
a first treatment portion comprising:
a main channel;
at least one reservoir;
a channel connecting said main channel and said reservoir; and
at least one flow control portion for setting an opening and closing of said channel, and
a second treatment portion comprising:
a main channel;
at least five reservoirs;
a channel connecting said main channel and said reservoir;
a damming portion provided to said channel for damming a liquid in said reservoir;
a trigger channel in communication with said channel close to said damming portion, said trigger channel being for guiding said liquid to said damming portion;
at least four liquid switch portions each comprising said damming portion and said trigger channel;
at least one closing switch for closing said channel;
at least two lag channels provided to said trigger channel or said channel; and
at least two flow control portions for setting an opening and closing of said channel or said trigger channel, wherein:
the analysis portion comprises:
at least eight first treatment portions; and
at least two second treatment portion, with either one of the first and the second treatment portions having at least one reservoir holding a reagent, wherein:
when said first treatment portion has the reagent,
the reagent is necessary for determining any one, or two or more test items selected from the group consisting of:
alkaline phosphatase;
lactate dehydrogenase;
total protein;
albumin;
agent for zinc phosphate turbidity test;
agent for thymol turbidity test;
choline esterase; and
total bilirubin; and
when said second treatment portion has the reagent,
the reagent is necessary for determining any one, or two or more test items selected from the group consisting of:
HBs antibody; and
HCV antibody.

32. A chip having an analysis portion comprising:
at least seven first treatment portions each of which includes:
a main channel;
at least one reservoir;
a channel connecting said main channel and said reservoir;
at least one flow control portion for setting an opening and closing of said channel,
wherein at least one of the seven first treatment portions has at least one reservoir in which reagent has been stored,
the reagent being necessary for determining any one, or two or more test items selected from the group consisting of:
total protein;
albumin;
urea nitrogen;
creatinine;
sodium ion;
potassium ion; and
chlorine ion.

33. A chip having an analysis portion comprising:
a first treatment portion comprising:
a main channel;
at least one reservoir;
a channel connecting said main channel and said reservoir; and
at least one flow control portion for setting an opening and closing of the channel, and
a second treatment portion comprising:
a main channel;
at leas five reservoirs;
a channel connecting said main channel and said reservoir;
a damming portion provided to said channel for damming a liquid in said reservoir;
a trigger channel in communication with the channel close to said damming portion, said trigger channel being for guiding said liquid to said damming portion;
at least four liquid switch portions each comprising said damming portion and said trigger channel;
at least one closing switch for closing said channel;
at least two lag channels provided to said trigger channel or said channel; and
at least two flow control portions for setting an opening and closing of said channel or said trigger channel, wherein:
said analysis portion comprises:
at least five first treatment portions; and
at least two second treatment portion, with either one of the first and the second treatment portions having at least one reservoir holding a reagent, wherein:
when said first treatment portion has the reagent,
the reagent is necessary for determining any one, or two or more test items selected from the group consisting of:
creatinine;
sodium ion;
potassium ion; and
chlorine ion, and
when said second treatment portion has the reagent,
the reagent is necessary for determining any one, or two or more test items selected from the group consisting of:
agent for determining renin activity; and
aldosterone.

34. A chip having an analysis portion comprising:
a first treatment portion comprising:
a main channel;
at least one reservoir;
a channel connecting said main channel and said reservoir; and
at least one flow control portion for setting an opening and closing of said channel, and
a second treatment portion comprising:
a main channel;
at leas five reservoirs;
a channel connecting said main channel and said reservoir;
a damming portion provided to said channel for damming a liquid in said reservoir;
a trigger channel in communication with said channel close to said damming portion, said trigger channel being for guiding said liquid to said damming portion;
at least four liquid switch portions each comprising said damming portion and said trigger channel;
at least one closing switch for closing said channel;
at least two lag channels provided to said trigger channel or said channel; and
at least two flow control portions for setting an opening and closing of said channel or said trigger channel, wherein:
the analysis portion comprises:
at least two first treatment portions;
at least two second treatment portion with either one of the first and the second treatment portions having at least one reservoir holding a reagent, wherein:
when said first treatment portion has the reagent,
the reagent is necessary for determining any one, or two or more test items selected from the group consisting of:
serum iron; and
ferritin, and
when said second treatment portion has the reagent,
the reagent is necessary for determining any one, or two or more test items selected from the group consisting of:
vitamin B12; and
folic acid.

35. A chip having an analysis portion comprising:
a first treatment portion including:
a main channel;
at least one reservoir;
a channel connecting the said main channel and said reservoir; and
at least one flow control portion for setting an opening and closing of said channel, wherein:
said first treatment portion holds a reagent necessary for the detection of uric acid.

36. A chip having an analysis portion comprising:
at least three second treatment portions each comprising:
a main channel;
at least five reservoirs;
a channel connecting said main channel and said reservoir;
a damming portion provided to said channel for damming a liquid in said reservoir;
a trigger channel in communication with said channel close to said damming portion, said trigger channel being for guiding said liquid to the damming portion;
at least four liquid switch portions each comprising said damming portion and said trigger channel;
at least one closing switch for closing said channel;
at least two lag channels provided to said trigger channel or said channel; and
at least two flow control portions for setting an opening and closing of said channel or said trigger channel, wherein:
at least one of three second treatment portions has one reservoir for holding a reagent, the reagent being necessary for determining any one, or two or more test items selected from the group consisting of:
triiodothyronine;
thyroxine; and
thyroid gland stimulating hormone.

37. A chip having an analysis portion comprising:
a second treatment portion comprising:
a main channel;
at leas five reservoirs;
a channel connecting said main channel and said reservoir;
a damming portion provided to the channel for damming a liquid in said reservoir;
a trigger channel in communication with said channel close to said damming portion, said trigger channel being for guiding said liquid to said damming portion;
at least four liquid switch portions each comprising said damming portion and said trigger channel;
at least one closing switch for closing said channel;
at least two lag channels provided to said trigger channel or said channel; and
at least two flow control portions for setting an opening and closing of said channel or said trigger channel, wherein:
said second treatment portion holds a reagent necessary for the detection of cortisol.

38. The chip according to claim 27, further comprising a control analysis portion for analyzing a standard solution having the same configuration with said analysis portion for analyzing a sample.
